(19) United States
(12) Patent Application Publication
(10) Pub. No.: US 2026/0327724 A1
(43) Pub. Date: Oct. 22, 2026

(54) COMPOSITIONS AND METHODS FOR ANALYSIS OF HPV

(71) Applicant: The Regents of the University of Michigan, Ann Arbor, MI (US)

(72) Inventors: Chandan Bhambhani, Ann Arbor, MI (US); John C. Brenner, Ann Arbor, MI (US); Paul Swiecicki, Ann Arbor, MI (US); Muneesh Tewari, Ann Arbor, MI (US)

(21) Appl. No.: 19/482,676

(22) PCT Filed: May 9, 2024
(86) PCT No.: PCT/US2024/028566

§ 371 Date: Nov. 7, 2025

Related U.S. Application Data
(60) Provisional application No. 63/500,979, filed on May 9, 2023.

(51) Int. Cl. C12Q 1/70

(52) U.S. Cl. CPC C12Q 1/708; C12Q 2600/16

(57) ABSTRACT

Provided herein are compositions, methods, and kits for detecting, amplifying, and/or quantifying analytes from various samples. For example, the disclosure provides a compositions, methods, and kits capable of detecting, amplifying, and/or quantifying human papilloma virus (HPV) related cancer circulating tumor DNA (ctDNA).

### CROSS REFERENCE TO RELATED APPLICATION

This application claims priority to and the benefit of U.S. Provisional patent application Ser. No. 63/500,979 filed May 9, 2023, which is incorporated herein by reference in entirety and for all purposes.

### SEQUENCE LISTING

The text of the computer readable sequence listing filed herewith, titled “41733-601_SEQUENCE_LISTING”, created May 9, 2024, having a file size of 163,578 bytes, is hereby incorporated by reference in its entirety.

### FIELD

Provided herein are compositions, methods, and kits for detecting, amplifying, and/or quantifying analytes from various samples. For example, the disclosure provides a compositions, methods, and kits capable of detecting, amplifying, and/or quantifying human papilloma virus (HPV) related cancer circulating tumor DNA (ctDNA).

### BACKGROUND

HPV infection is associated with a number of cancers, including cancers of the cervix, vulva, vagina, penis, anus, and oropharynx and there are a number of unmet detection, surveillance, and treatment needs. For example, HPV-related oropharyngeal squamous cell carcinoma (HPV+ OPSCC) is a growing epidemic that, despite availability of an HPV vaccine, will continue to be a worsening public health problem for decades because of the relatively low adoption of the vaccine in the US [1-9]. While patients diagnosed with low stage disease have an excellent prognosis, more than a quarter of patients with HPV+ OPSCC present with high-risk disease and are likely to have disease recurrence [10]. Patients with recurrent HPV+ OPSCC have a dismal 5-year survival of only 3-26% [11-13].

Data from other solid tumors indicate that during post-treatment surveillance (PTS), recurrences detected by biofluid-based biomarkers (i.e., “biochemical recurrence”) prior to the appearance of CT scan-detectable disease, have the potential to be cured with salvage therapy [14-19]. Consistent with this, there is improved survival in locally recurrent OPSCC when disease volume is smaller [20], suggesting that earlier detection of HPV+ OPSCC recurrence would improve outcomes. However, the current standard-of-care for HPV+ OPSCC PTS is periodic clinical examination, which requires recurrent tumors to be relatively large for detection. Unfortunately, CT scan-based PTS has been tried but had low sensitivity, high false positive rate, and did not increase survival [21,22]. Thus, there are currently no biomarker testing approaches shown to improve outcomes in PTS of HPV+ OPSCC and improved HPV-related cancer detection strategies are needed.

### SUMMARY

Provided herein are compositions, methods, and kits for detecting, amplifying, and/or quantifying analytes from various samples. For example, the disclosure provides a compositions, methods, and kits capable of detecting, amplifying, and/or quantifying human papilloma virus (HPV) related cancer circulating tumor DNA (ctDNA).

Some of the main aspects provided herein are summarized below. Additional aspects are described in the Brief Description of the Drawings, Definitions, Detailed Description, Experimental, and Claims sections of this disclosure. The description in each section of this patent disclosure, regardless of any heading or sub-heading titles, is intended to be read in conjunction with all other sections. Furthermore, the various embodiments described in each section of this disclosure can be combined in various different ways, and all such combinations are intended to fall within the scope of the present invention.

In some aspects, provided herein are compositions for use in a multi-probe HPV ctDNA assay. In some embodiments, the composition includes one or more modified oligonucleotide primer/probe sets. In some embodiments, the primer/probe sets are used in the amplification and quantification of DNA. In some embodiments, the DNA is HPV-related cancer (e.g., HPV+ OPSCC). In some embodiments, the primer/probe sets are designed to amplify and/or detect one or more target regions of a high-risk HVP type (e.g., HPV16, HPV18, HPV31, HPV33, and/or HPV39). In some embodiments, the target sets are selected from the group consisting of: SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36; SEQ ID NO: 52; SEQ ID NO: 53; SEQ ID NO: 54; SEQ ID NO: 56. In some embodiments, the primer/probe sets comprise one or more oligonucleotides that are selected from the group consisting of: SEQ ID NOs: 1-27 and SEQ ID NOs: 37-51. In some embodiments, at least a triad of the primer/probe sets are selected from the group consisting of: Set 1: SEQ ID NO: 1, SEQ ID NO: 2, SEQ ID NO: 3; Set 2: SEQ ID NO: 4, SEQ ID NO: 5, SEQ ID NO: 6; Set 3: SEQ ID NO: 7, SEQ ID NO: 8, SEQ ID NO: 9; Set 4: SEQ ID NO: 10, SEQ ID NO: 11, SEQ ID NO: 12; Set 5: SEQ ID NO: 13, SEQ ID NO: 14, SEQ ID NO: 15; Set 6: SEQ ID NO: 16, SEQ ID NO: 17, SEQ ID NO: 18; Set 7: SEQ ID NO: 19, SEQ ID NO: 20, SEQ ID NO: 21; Set 8: SEQ ID NO: 22, SEQ ID NO: 23, SEQ ID NO: 24; Set 9: SEQ ID NO: 25, SEQ ID NO: 26, SEQ ID NO: 27; Set 10: SEQ ID NO: 37, SEQ ID NO: 38, SEQ ID NO: 39; Set 11: SEQ ID NO: 40, SEQ ID NO: 41, SEQ ID NO: 42; Set 12: SEQ ID NO: 43, SEQ ID NO: 44, SEQ ID NO: 45; Set 13: SEQ ID NO: 46, SEQ ID NO: 47, SEQ ID NO: 48; Set 14: SEQ ID NO: 49, SEQ ID NO: 50, SEQ ID NO: 51. In some embodiments, the primer/probe set(s) is selected from the group consisting of: a forward primer having at least 90% sequence identity to any one or more of: SEQ ID NOs: 1, 4, 7, 10, 13, 16, 19, 22, 25, 37, 40, 43, 46, 49; a reverse primer having at least 90% sequence identity to any one or more of: SEQ ID NOs: 2, 5, 8, 11, 14, 17, 20, 23, 26, 39, 42, 45, 48, 51; a probe comprising a detectable label, wherein the forward primer and the reverse primer anneal to any one or more of: SEQ ID NOs: 3, 6, 9, 12, 15, 18, 21, 24, 27, 38, 41, 44. 47, 50. In some embodiments, the first set of oligonucleotides comprises SEQ ID NOs: 1-15 or SEQ ID NOs: 37-45. In some embodiments, the second set of oligonucleotides comprises SEQ ID NOs: 16-27 or SEQ ID NOs: 46-51. In some embodiments, a probe used with any of the primer sets comprises a sequence that hybridizes to a target sequence between or overlapping the primer sequences. In some embodiments, the primer/probe set does not have greater than an eight-base pair stretch of bases annealing as a homodimer, hairpin, or heterodimer. In some embodiments, the first set of oligonucleotides comprises a first detectable label (e.g., FAM) and the second set of oligonucleotides comprises a second detectable label (e.g., VIC). In some embodiments, the second labeled set of oligonucleotides targets a viral E6/E7 junction wherein the junction is highly represented in HPV16 ctDNA. In some embodiments, the first labeled set of oligonucleotides passes a two-fold criterion and does not alter the background in a second label channel when compared to a single probe set of oligonucleotides. In some embodiments, the second labeled set of oligonucleotides is selected from the group with SEQ ID Nos: 2, 5, 8, 11, 14, 17, 20, 23, 26, 39, 42, 45, 48, 51 and the first labeled set of oligonucleotides is selected from the group with SEQ ID Nos: SEQ ID NOs: 1, 4, 7, 10, 13, 16, 19, 22, 25, 37, 40, 43, 46, 49. In some embodiments, the second labeled set of oligonucleotides targets a viral E6/E7 junction and said junction is highly represented in HPV16 ctDNA and the first labeled set of oligonucleotides passes a two-fold criterion and does not alter the background in a second label channel when compared to a single probe set of oligonucleotides.

In some embodiments, provided herein are methods of detecting and/or quantifying HPV-associated nucleic acid (e.g., viral nucleic acid and/or ctDNA). In some embodiments, provided herein are methods for detecting HPV subtype 16 (HPV16) ctDNA, HPV subtype 18 (HPV18) ctDNA, HPV subtype 31 (HPV31) ctDNA, and/or HPV subtype 39 (HPV39). In some embodiments, the methods comprise contacting a sample comprising viral nucleic acid or ctDNA with a set of oligonucleotides. In some embodiments, the set of oligonucleotides comprises a forward primer, a reverse primer, and a probe. In some embodiments, the methods comprise contacting a sample with a composition of any one or more of the above listed compositions. In some embodiments, the sample is amniotic fluid, ascites, bile, breast milk, breast milk colostrum, bronchoalveolar lavage fluid, cerebrospinal fluid, dialysate, eye aqueous humor, eye vitreous humor, feces, paracentesis, pericardial fluid, peritoneal, blood plasma, pleural, semen, blood serum, synovial fluid, tears, thoracentesis, blood, saliva, gargle, or urine, is derived from any such sample, although any other sample type may be used. In some embodiments, the contacting a sample comprises: providing one or more of the modified oligonucleotide primer/probe sets; fractionating a plurality of HPV DNA from the sample into droplets at a concentration wherein only 0 or 1 molecule of the DNA is present in each droplet; amplifying HPV DNA in each droplet with one or more primer/probe sets to produce amplicon signals; and detecting in each droplet any amplicon signals. In some embodiments, the DNA is fractionated into micro-droplets by emulsification. In some embodiments, the DNA is amplified using a nucleic acid amplification method. In some embodiments, the nucleic acid amplification method comprises polymerase chain reaction (PCR), loop mediated isothermal amplification (LAMP), nucleic acid sequence-based amplification (NASBA), strand displacement amplification (SDA), or multiple displacement amplification (MDA). In some embodiments, contacting comprises conducting a quantitative PCR (qPCR) assay. In some embodiments, a qPCR assay comprises a digital PCR assay. In some embodiments, a digital PCR assay comprises a droplet digital PCR (ddPCR) assay. In some embodiments, the sample comprises a ctDNA HPV target nucleic acid and wherein one or more of said oligonucleotides hybridize to said HPV target nucleic acid. In some embodiments, the assay has a limit of detection of <1 genome equivalent of HPV DNA.

In some embodiments, the HPV nucleic acid or a product derived therefrom (e.g., an amplicon) is sequenced. In some embodiments, the sequencing technique is a next generation sequencing technique. The term “next generation sequencing” refers to highly parallelized methods of performing nucleic acid sequencing and comprises the sequencing-by-synthesis or sequencing-by-ligation platforms (e.g., employed by Illumina, Life Technologies, Pacific Biosciences and Roche, etc.). Next generation sequencing methods may also include, but not be limited to, nanopore sequencing methods such as offered by Oxford Nanopore or electronic detection-based methods such as the Ion Torrent technology commercialized by Life Technologies. In some embodiments, one or more of the primers described herein further comprises an additional sequence (e.g., barcode, adapter, etc.) that finds use in sequencing library preparation, sequencing, and analysis. Suitable nucleic acid sequencing techniques include, but are not limited to, sequencing by synthesis (see e.g., Meyer and Kircher, “Illumina sequencing library preparation for highly multiplexed target capture and sequencing,” Cold Spring Harbor Protocols 2010 (6)); single-molecule real-time sequencing (see e.g., Levene et al., “Zero-Mode Waveguides for Single-Molecule Analysis at High Concentrations,” Science. 299 (5607): 682-6 (2003)); ion semiconductor sequencing (see e.g., Rusk, “Torrents of sequence,” Nat. Methods 8, 44 (2011)); pyrosequencing (see e.g., Wicker et al., “454 sequencing put to the test using the complex genome of barley,” BMC Genomics, 7:275, 2006); sequencing by ligation (SOLID sequencing) (see e.g., Margulies et al., “Genome sequencing in microfabricated high-density picolitre reactors,” Nature, 437:376-80 (2005)); nanopore sequencing (see e.g., Goodwin et al., “Oxford Nanopore sequencing, hybrid error correction, and de novo assembly of a eukaryotic genome,” Genome Res., 25 (11): 1750-6 (2015)); chain termination sequencing (Sanger sequencing) (see e.g., Sanger et al., “DNA sequencing with chain-terminating inhibitors, “Proceedings of the National Academy of Sciences of the United States of America, 74 (12): 5463-5467 (1977)); and sequencing with mass spectrometry (see e.g., Edwards et al., “Mass-spectrometry DNA sequencing,” Mutation Research, 573 (1-2): 3-12 (2005)).

In some embodiments, provided herein are methods of detecting HPV nucleic acid and/or ctDNA and, in some embodiments, further comprising the step of treating a subject with cancer surveillance, therapy, or other intervention if HPV is detected in a sample. In some embodiments, the cancer treatment comprises an imaging technique, a clinical examination, excision treatment, cryotherapy, thermal ablation, radiotherapy, chemotherapy, and/or salvage therapy. In some embodiments, the treatment is provided prior to appearance of CT scan-detectable.

In some embodiments, provided herein are methods of detecting HPV nucleic acid and/or ctDNA and, in some embodiments, further comprising the step of treating a subject with an HPV vaccine if HPV is not detected in a sample.

In some embodiments, provided herein are kits. In some embodiments, the kits may comprise one or more sets of oligonucleotides, as described herein. In some embodiments, the kits may further comprise reagents necessary, useful, or sufficient to purify, isolate, detect and/or quantify HPV nucleic acid and/or ctDNA. For example, the kits may further comprise amplification reagents, including buffers and enzymes. In some embodiments, the kit may further comprise control samples, if needed or desired. In some embodiments, the kit may comprise solid surfaces (e.g., beads) comprising capture reagents (e.g., oligonucleotides) specific for target ctDNA. In some embodiments, the kit can further include containers for holding or storing a sample, reagents, or reaction mixtures (e.g., a container or cartridge for a plasma sample, a container for a cell free DNA sample, etc.). In some embodiments, the kit can also include one or more instruments for assisting with obtaining or manipulating a test sample, such as a syringe. Where appropriate, the kit can contain reaction vessels, mixing vessels, and other components that facilitate the preparation of reagents (e.g., a container for mixing reagents for PCR). In some embodiments, the kit may further comprise instructions for use of the kit. Instructions included in kits can be affixed to packaging material or can be included as a package insert or can be viewed or downloaded from a particular website that is recited as part of the kit packaging or inserted materials. While the instructions are typically written or printed materials, they are not limited to such. Any medium capable of storing such instructions and communicating them to an end user is contemplated by this disclosure. Such media include, but are not limited to, electronic storage media (e.g., magnetic discs, tapes, cartridges, chips), optical media (e.g., CD ROM), and the like. As used herein, the term “instructions” can include the address of an Internet site that provides the instructions.

### BRIEF DESCRIPTION OF THE DRAWINGS

FIGS. 1A-1B show the design, development, and experimental testing of multiple primer/probe sets targeting the HPV16 genome. FIG. 1A shows a flowchart outlining the steps for design and development of the HPV16 multi-probe assay. Briefly, computational analysis was used to scan the entire HPV16 genome for primer and probe binding regions complying with the primer/probe design criteria to select combinations predicted to specifically bind the HPV16 genome. A schematic based on The Papillomavirus Episteme (PaVE) coordinates depicts the HPV16 genomic regions targeted by primer/probe combinations at various stages of selection. Of the forty-nine (49) primer/probe sets selected from computational screens (forward primer=green, reverse primer=red), eighteen (18) non-overlapping sets were selected with examples circled and one (1) primer/probe set (see primer/probe set #4 in FIG. 1B) overlapping with another primer set at the E6/E7 junction was selected based on higher GC content, making a total of nineteen (19) primer/probe sets for functional screens. (Note: ‘SP assay’=previously validated, single-probe assay with a primer/probe set targeting a seventy-seven (77) bp region of HPV16; [29]). FIG. 1B shows a ddPCR droplet profile displaying the FAM signal (blue) for the nineteen (19) selected primer/probe candidates, tested in duplicates at final primer/probe concentration of 900 nM/250 nM using 400 Genome Equivalent (GEs) of HindIII digested HPV16 cell line UM-SCC-104 genomic DNA (gDNA) or 200,000 GEs of HindIII digested non-HPV human genomic DNA (hgen DNA) as template. The threshold (pink line) was kept constant for all primer/probe combinations except for primer/probe set #17 and primer/probe set #18, where the signal intensity was not high compared to the background (gray). HPV16 copies detected (average of duplicates) with each primer/probe combination compared to the SP assay (orange) are tabulated at the bottom. In the case of primer/probe combination #14, the background could not be distinguished from the FAM signal. No signal was observed with the negative control non-HPV hgen DNA as template, for any of the primer/probe combinations.

FIGS. 2A-2C show a primer/probe pool selection for multi-probe assay development. FIG. 2A shows the pooling schema for development of a multi-probe assay using primer/probe sets targeting different regions of HPV16 and selected based on a high signal intensity compared to background during screening. The schematic shows a combination of 3, 5, 10, or 14 primer/probe sets, termed 3-Pool, 5-Pool, 10-Pool, and 14-Pool, respectively

FIG. 2B shows ddPCR droplet profile displaying the FAM signal (blue) in a single-plex assay for 5 primer/probe sets individually (#4, #10, #13, #15 and #16), as well as for 3-Pool, 5-Pool, 10-Pool, and 14-Pool, with a concentration of 450 nM of each primer and 125 nM of each probe. In the case of 3-Pool and 5-Pool, separation of the signal from the background (gray) allowed for the calculation of HPV16 copies but in the case of 10-pool and 14-pool the signal overlapped with the background and HPV16 copies could not be calculated. The HPV16 copies obtained for individual primer/probe combinations, 3-Pool and 5-Pool with sheared UM-SCC-104 gDNA (500 GEs) or water control as template are tabulated at right. Data shown is representative of different primer/probe concentrations tested to improve signal intensity to background ratios for the pools (FIG. 7). FIG. 2C shows VIC labeled probes #2, #4, #13, #15, and #16 which were tested in various combinations of four (4) primer/probe sets to screen for a 4-Pool using sheared UM-SCC-104 gDNA template (100 GEs) at final primer/probe concentration of 450 nM/125 nM each. The droplet profile shows signal intensity (green) and the background (gray). HPV16 copies are listed and 4-Poolv20 (highlighted in light green) was chosen for further experimental evaluation.

FIG. 3 shows the screening and selection of a dual-colored multi-probe pool. A 2-D ddPCR plot displays droplet profiles of single-probe FAM assay #6 and different 5-Pool combinations containing FAM labeled probes (blue) screened with 4-Poolv20 (#2+ #4+ #13+ #15) containing VIC labeled probes (green) at final primer/probe concentration of 450 nM/125 nM each, using sheared UM-SCC-104 gDNA as template (50 GEs). HPV16 copies along with fold enhancement of the signal measured with the 5-Pool FAM combinations over the single-probe (#6) assay are tabulated at the bottom. The best separation of the background (gray) from signal (blue) was observed in 5-Poolv39FAM (#5+ #6+ #7+ #16+ #19) (panel G; highlighted in light red) when comparing the FAM labeled single-probe (#6) assay to the different 5-Pool FAM combinations. The threshold of 6200 for 4-Poolv20VIC was kept constant to ensure that the FAM pool was not changing the background intensity of the 4-Poolv20VIC in the case of dual-channel measurement. In cases where FAM pools altered the background (e.g., 5-Poolv35 and 5-Poolv41), HPV16 copies could not be determined and are listed as ‘nd.’

FIG. 4 shows the analytical validation of the dual-colored multi-probe CHAMP-16 Assay. Panel A shows a linearity plot of ddPCR results from a 2-fold dilution series (average of 3 replicates) to determine the Limit of Detection (LoD) and reportable range of HPV16 circulating tumor DNA (ctDNA) using a template comprising 9 synthetic HPV16 dsDNA duplexes corresponding to the genomic regions targeted by the 9 primer/probe combinations pooled in the CHAMP-16 assay. The synthetic HPV16 target DNA pool and sheared UM-SCC-104 gDNA were tested in a background of sheared hgen matrix (~44,000 diploid GEs per well) and the expected copies (x-axis) were plotted against the measured copies (y-axis). Panel B shows a linearity plot of ddPCR results from a 2-fold dilution series (average of 3 replicates) to determine the Limit of Detection (LoD) and reportable range of HPV16 ctDNA using sheared UM-SCC-104 gDNA as template. The synthetic HPV16 target DNA pool and sheared UM-SCC-104 gDNA were tested in a background of sheared hgen matrix (~44,000 diploid GEs per well) and the GEs tested (x-axis) were plotted against the measured copies (y-axis). The LoD was analyzed and calculated to be 4.1 copies per 20 μl reaction. No non-specific signal was observed when only sheared hgen DNA (~44,000 diploid GEs per 20 μl reaction) was used as a non-HPV template (n=61). Panel C shows % CV plotted for measured copies of HPV16 at different dilutions (y-axis) versus the expected copies (converted to log 10) of synthetic pool of 9 DNA targets tested (x-axis). Panel D shows % CV plotted for measured copies of HPV16 at different dilutions (y-axis) versus the number of GEs (converted to log 10) of sheared UM-SCC-104 gDNA tested (x-axis). An arbitrary 20% threshold is indicated by a dashed line.

FIG. 5 shows a case study comparing the CHAMP-16 assay to the conventional SP assay for early detection of recurrence. Plasma samples collected from a biopsy-confirmed HPV+ OPSCC patient undergoing chemoradiotherapy (CRT) were analyzed for HPV16 ctDNA using the SP assay (orange triangles) and CHAMP-16 assay (green circles). Number of HPV16 copies detected (y-axis) were plotted as a cumulative of duplicate plasma cfDNA samples tested from blood drawn over several months (x-axis). PET (+) indicates a positive PET scan, whereas PET (−ve) indicates a negative PET scan with respect to detecting presence of cancer. Closed symbols represent HPV16 molecules detected above LoD (black dotted line), and open symbols represent copies that were below the LoD. Notably, the CHAMP-16 assay was able to detect HPV16 ctDNA signal ~20 months prior to clinical recurrence and signal detection by the SP assay.

FIG. 6 shows a computational screen for primer/probe sets targeting the HPV16 genome. A schematic based on the PaVE coordinates depicts the HPV16 genomic regions targeted by 292 primer/probe combinations (forward orientation oligonucleotide sequences in green and reverse orientation oligonucleotide sequences in red) selected by computational analysis using melting temperature, amplicon length and % GC criteria. Various other criteria (e.g., auto-dimer formation, potential non-specific amplification, etc.) were used for further screening, which led to selection of 49 primer/probe combinations.

FIG. 7A-7C shows the determination of the optimal primer/probe combinations and concentration for a pool. Droplet profile displaying the FAM signal (blue) in a single-plex assay for 5 primer/probe sets individually (#4, #10, #13, #15 and #16), as well as for 3-Pool, 5-Pool, 10-Pool and 14-Pool (pooled as per the scheme in FIG. 2A) using UM-SCC-104 gDNA (500 GEs) as template. Note that at 50 or 60 PCR cycles, the background (gray) increased significantly with increasing number of pooled primer/probe sets tested. FIG. 7A shows primer/probe sets tested at a concentration of (A) 900 nM each primer and 250 nM each probe. FIG. 7B shows primer/probe sets tested at a concentration 450 nM each primer and 125 nM each probe. FIG. 7C shows primer/probe sets tested at a concentration of 180 nM each primer and 50 nM each probe. In the case of 3-Pool and 5-Pool, the signal intensity over the background allowed for the calculation of HPV16 copies but in the case of 10-Pool and 14-Pool the signal overlapped with the background and HPV16 copies could not be calculated. The decrease in signal was not due to lower droplet counts of the primer/probe concentrations shown in FIG. 7B and FIG. 7C, although droplet counts were negatively impacted for 5-Pool, 10-Pool and 14-Pool at the highest primer/probe concentration shown in FIG. 7A (i.e., 900 nm/450 nm).

FIG. 8 shows representative droplet profiles demonstrating the criterion used for selection of 5-Pool combinations. Representative data of 5-Pool combinations showing examples of rejected pools (R1, R2, and R3) which did not undergo further testing, and accepted pools (A1, A2 and A3) selected for further testing based on the 2-fold criterion of signal intensity (blue) over the background (gray). #SP assay refers to the primer/probe set corresponding to the previously published single-probe HPV16 assay.

FIG. 9 shows the screening and selection of 5-Pool combinations containing FAM labeled probes. Droplet profiles of 8 representative 5-Pool combinations that were tested, with two of those rejected (v18 and v20) as they included the primer/probe set #10 targeting the E2 gene. 5-Poolv2 highlighted in light blue was chosen based on 2-fold higher signal intensity (darker blue) compared to the background (gray) and highest number of HPV16 copies detected. Sheared UM-SCC-104 gDNA or water was used as the template for ddPCR with the final primer/probe concentration of 180 nM/50 nM each.

FIG. 10 shows a comparison of the signal intensity of the FAM labeled and VIC labeled probes in a single-probe assay. Droplet profiles displaying the signal intensity of individual primer/probe sets (#2 or #4 or #13 or #15 or #16) in a single-probe assay with either FAM (blue) or VIC (green) labeled probes using sheared UM-SCC-104 gDNA (100 GEs) as template for ddPCR at a final primer/probe concentration of 900 nM/250 nM each. The signal amplitude for each channel is listed in the table below along with the approximate fold signal intensity over background calculated based on the amplitude. A significant reduction in the signal intensity was observed with the VIC labeled probes when compared to the FAM labeled probes.

FIG. 11 shows a comparison of the signal intensity of the FAM labeled and VIC labeled probes as a multi-probe pool. Droplet profiles displaying the signal intensity of a primer/probe pool (5-Poolv2) containing primer/probe sets #2, #4, #13, #15, and #16, with probes labeled with either FAM (blue) or VIC (green) fluorophore. Sheared UM-SCC-104 gDNA template at 20 or 100 GEs was used for ddPCR with final primer/probe concentration of 180 nM/50 nM each. The signal amplitude for each channel is listed in the table below along with the approximate fold signal intensity over background calculated based on the amplitude. A significant reduction in the separation of the signal from the background was observed for the VIC labeled version of the pool when compared to the FAM labeled version.

FIG. 12 shows a determination of the optimal annealing temperature for the CHAMP-16 assay. The assay was tested across a range of annealing temperatures (64° C. to 56° C.) using sheared UM-SCC-104 gDNA (50 GEs) as the template. Representative droplet profiles (FAM (blue) and VIC (green)) at different annealing conditions tested are shown with the HPV16 copies listed in the table below. Significantly lower FAM and VIC positive droplets were recorded at temperatures greater than 60° C. suggesting incomplete amplification as indicated by the representative droplet profile (brackets) at 63° C. and HPV16 copies listed. Amplification was optimal at 59° C. as seen in the droplet plots and no further improvement was observed at lower temperatures tested including the least one tested (56° C.) as indicated by the HPV16 copies. Hence 59° C. was chosen as the annealing temperature. Additionally, no signal was observed at this annealing temperature when tested with non-HPV hgen DNA as template (44,000 diploid GEs).

FIG. 13 shows a comparison of the analytical sensitivity of SP assay and multi-probe CHAMP-16 assay. HPV16 genomic regions depicting the position of 9 primer combinations (forward primer in green and reverse primer in red) pooled in the CHAMP-16 assay relative to the position of the primer/probe combination in the SP assay. The table lists HPV16 copies detected by the CHAMP-16 assay and SP assay using sheared UM-SCC-104 gDNA as the template. CV=Coefficient of Variation.

FIG. 14 shows the individual primer/probe sets compared for respective target amplification as a single-probe assay and as a multi-probe pool. HPV16 copies measured by the 9 primer/probe combinations were compared, when tested as an individual primer/probe assay or as a pool in the CHAMP-16 assay using ddPCR. Synthetic DNA corresponding to each of the 9 targeted regions of HPV16 was spiked into a hgen DNA matrix of 44,000 diploid GEs and used as template (top table). Similar analysis was performed with the 9 synthetic DNA targets pooled and tested as a contrived sample at two different dilutions (bottom table).

FIG. 15 shows comprehensive patient demographics and HPV16 ctDNA testing results. Demographics for 41 patients with HPV+ OPSCC were tested with the SP assay and CHAMP-16 assay. HPV16 copies (average of duplicates) from plasma cfDNA analyzed for each assay and the corresponding plasma amount tested per replicate are listed. Patient samples #1-#21 were known to be positive for HPV16 ctDNA based on prior analysis using the SP assay. Samples from patients #22-#41 corresponded to residual biobanked samples from historical clinical trials collected at baseline (i.e., prior to initiating treatment). Of note, these samples were collected in previous studies and processed differently than those from Patients #1-#21, including having undergone one or more freeze-thaw cycles. Furthermore, although tumor tissue from patients #22-#41 was confirmed to be p16(+), HPV16 positivity was not separately established. For four patients, HPV16 ctDNA copies (red) were detected with the CHAMP-16 assay but not with the SP assay. Samples which tested negative in either SP or CHAMP-16 assay(s) are shaded in dark gray.

FIG. 16 shows clinical specificity control groups. Eleven patients with HPV negative cancer (patients #1-#11) and 3 non-cancer control subjects (patients #12-#14) were tested using the SP and CHAMP-16 assays and no signal was detected. This is consistent with the analytical data for the CHAMP-16 assay, which was negative when tested with 44,000 diploid GEs of non-HPV human reference DNA.

FIG. 17 patient demographics and comparison of SP assay and CHAMP-16 assay results to reported NavDx assay values for HPV16 ctDNA. Plasma samples collected close in time to those sent for commercial NavDx analysis from 8 HPV+ OPSCC patients were tested using the CHAMP-16 assay and SP assay. For the 3 patients positive with the NavDx assay (patients #42-#44), a significant signal enhancement was observed with the CHAMP-16 assay compared to the commercial assay. Samples from 5 patients collected 3 months post-CRT (patients #45-#49), which did not show any HPV16 signal with the NavDx assay, also tested negative for HPV16 with both SP and CHAMP-16 assays.

FIG. 18 shows a longitudinal HPV16 ctDNA analysis for recurrence-patient #19. Plasma samples collected from a patient with biopsy-confirmed HPV+ OPSCC were tested using the CHAMP-16 assay. HPV16 and reference gene RPP30 copies have been tabulated here and presented as a timeline in FIG. 5.

FIG. 19 shows a longitudinal HPV16 ctDNA analysis of patient #20. Plasma samples collected from a patient with biopsy-confirmed HPV+ OPSCC were tested using the CHAMP-16 assay. HPV16 and reference gene RPP30 copies have been tabulated here. Although the HPV16 copies were very high at baseline, after surgical resection the assay was negative for HPV16 at all subsequent time points evaluated, in contrast to patient #19 who experienced cancer recurrence (FIG. 5 and FIG. 18).

FIG. 20 shows clinical sample testing using the CHAMP-16 assay. Plasma samples from 21 patients with p16+ HNSCC, known to be HPV16 ctDNA positive using the conventional SP assay, were analyzed using both the SP assay and the CHAMP-16 assay. HPV16 copies (average of duplicates) from plasma cfDNA analyzed for each assay are listed. All patients tested positive in both assays; CHAMP-16 assay signal enhancement averaged 6.6-fold higher than the SP assay and is highlighted in green.

FIG. 21 shows a comparison of CHAMP-16 performance with SP assay and commercially available NavDx assay. Plasma samples from 3 patients with HPV+ OPSCC reported to have positive HPV16 ctDNA values using the NavDx assay were tested using the SP assay and CHAMP-16 assay. HPV16 copies (average of duplicates) detected in plasma cfDNA are listed and values extrapolated to one mL of plasma for comparison to values reported by the NavDx assay. Values observed with the SP assay were similar to those reported from NavDx testing, while a significant enhancement in signal was observed using the CHAMP-16 assay. Blood draws were carried out at the same time for all assays, except for patient #42 where the blood draw for NavDx testing was seven days prior to the pre-treatment draw used for SP and CHAMP-16 assays. Notably, 5 samples collected from patients at 3 months post-chemoradiation therapy (CRT) that had no signal with the NavDx assay also tested negative for HPV16 ctDNA with both SP and CHAMP-16 assays (FIG. 16), supporting the conclusion that signal enhancement observed with the CHAMP-16 assay was not due to non-specific signal.

FIG. 22 shows analytical validation-limit of detection of the CHAMP-hr assay for quantification of additional high-risk HPV types. Data from a 2-fold dilution series of ultra-short synthetic targets (45-55 bp) targeting the E6 gene of five known high risk HPV types (HPV18, HPV31, HPV33, HPV35 and HPV39) to define the detectable range. Expected copies of synthetic ultra-short HPV DNA corresponding to the high-risk types that were spiked into the ddPCR reaction (cumulative of 3 replicates). Limit of Detection (LOD) of the high-performance assay for each targeted HPV type is shown.

FIG. 23 shows RPP-30 reference gene and plant spike dual assay development: Comparison of the plant spike technical control and RPP-30 reference gene assays using ddPCR demonstrating that they perform equally well as single-plex and dual-plex assays.

FIG. 24 shows a computational analysis for HPV16. The image is a schematic of the selection process. Of the 49 primer/probe sets that passed the computational screens, 19 non-overlapping sets were tested experimentally, and 9 sets were chosen for the CHAMP-16 Assay pool. Of the 40 primer/probe sets which were not chosen for the CHAMP-16 Assay pool, 10 sets were tested experimentally and rejected, while the other 30 sets were not tested experimentally.

### DETAILED DESCRIPTION

Solid tumors are known to shed ctDNA that might be detectable in bodily fluids. Bodily fluid ctDNA analyses allow for non-invasive longitudinal monitoring of tumor specific genomic alterations. To date, a comprehensive prospective analysis of ctDNA method characteristics and predictive efficacy has not been performed in some HPV-related cancers (e.g., head and neck cancer).

Provided herein are compositions, methods, and kits for detecting, amplifying, and/or quantifying analytes from various samples. For example, the disclosure provides a compositions, methods, and kits capable of detecting, amplifying, and/or quantifying human papilloma virus (HPV) and related cancer circulating tumor DNA (ctDNA).

The compositions, methods, and kits described herein may employ any one or more or all of the oligonucleotides (e.g., primer/probe sets) described herein. These oligonucleotides may be used in combination with any other diagnostic assays (e.g., to detect other HPV types/strains, other cancer or pre-cancer biomarkers, inflammation, etc.).

For any of the compositions, methods, and kits described herein, any suitable sample type may be used. The sample may be obtained from the subject and subsequently used for any of the methods described herein. In some embodiments, the sample is obtained from the subject and an analyte is isolated, purified, and/or concentrated from the sample for use in the methods described herein.

Section headings as used in this section and the entire disclosure herein are merely for organizational purposes and are not intended to be limiting.

### 1. Definitions

Although any methods and materials similar or equivalent to those described herein can be used in the practice or testing of embodiments described herein, some preferred methods, compositions, devices, and materials are described herein. However, before the present materials and methods are described, it is to be understood that this invention is not limited to the particular molecules, compositions, methodologies, or protocols herein described, as these may vary in accordance with routine experimentation and optimization. It is also to be understood that the terminology used in the description is for the purpose of describing the particular versions or embodiments only and is not intended to limit the scope of the embodiments described herein.

Unless otherwise defined, all technical and scientific terms used herein have the same meaning as commonly understood by one of ordinary skill in the art to which this invention belongs. However, in case of conflict, the present specification, including definitions, will control. Further, unless otherwise required by context, singular terms shall include pluralities and plural terms shall include the singular. Accordingly, in the context of the embodiments described herein, the following definitions apply.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

As used herein and in the appended claims, the singular forms “a,” “an” and “the” include plural reference unless the context clearly dictates otherwise. Thus, for example, reference to “a domain” is a reference to one or more domains and equivalents thereof known to those skilled in the art, and so forth.

As used herein, the term “and/or” includes any and all combinations of listed items, including any of the listed items individually. For example, “A, B, and/or C” encompasses A, B, C, AB, AC, BC, and ABC, each of which is to be considered separately described by the statement “A, B, and/or C.”

The terms “include(s),” “having,” “has,” “can,” “contain(s),” and variants thereof, as used herein, are intended to be open-ended transitional phrases, terms, or words that do not preclude the possibility of additional acts or structures.

For any of the compositions, methods, and kits described herein, any suitable sample type may be used. The term “sample” means fluids (e.g., amniotic fluid, ascites, bile, breast milk, breast milk colostrum, bronchoalveolar lavage fluid, cerebrospinal fluid, dialysate, eye aqueous humor, eye vitreous humor, feces, paracentesis, pericardial fluid, peritoneal, blood (e.g., whole blood), blood product (e.g., plasma, serum), pleural, semen, synovial fluid, tears, thoracentesis, saliva, gargle, or urine, etc.), solids, tissues, and gases.

The term “saliva” or “saliva sample” means any sample containing saliva from a subject, including spit, an oral swab or sponge sample, a mouthwash rinse sample, etc.

The term “sequence identity” refers to the degree of which two polymer sequences (e.g., peptide, polypeptide, nucleic acid, etc.) have the same sequential composition of monomer subunits. The term “sequence similarity” refers to the degree with which two polymer sequences (e.g., peptide, polypeptide, nucleic acid, etc.) differ only by conservative and/or semi-conservative amino acid substitutions. The “percent sequence identity” (or “percent sequence similarity”) is calculated by: (1) comparing two optimally aligned sequences over a window of comparison (e.g., the length of the longer sequence, the length of the shorter sequence, a specified window, etc.); (2) determining the number of positions containing identical (or similar) monomers (e.g., same amino acids occurs in both sequences, similar amino acid occurs in both sequences) to yield the number of matched positions; (3) dividing the number of matched positions by the total number of positions in the comparison window (e.g., the length of the longer sequence, the length of the shorter sequence, a specified window); and (4) multiplying the result by 100 to yield the percent sequence identity or percent sequence similarity. For example, if peptides A and B are both 20 amino acids in length and have identical amino acids at all but 1 position, then peptide A and peptide B have 95% sequence identity. If the amino acids at the non-identical position shared the same biophysical characteristics (e.g., both were acidic), then peptide A and peptide B would have 100% sequence similarity. As another example, if peptide C is 20 amino acids in length and peptide Dis 15 amino acids in length, and 14 out of 15 amino acids in peptide D are identical to those of a portion of peptide C, then peptides C and D have 70% sequence identity, but peptide D has 93.3% sequence identity to an optimal comparison window of peptide C. For the purpose of calculating “percent sequence identity” (or “percent sequence similarity”) herein, any gaps in aligned sequences are treated as mismatches at that position.

Any peptides described herein as having a particular percent sequence identity or similarity (e.g., at least 70%) with a reference sequence ID number, may also be expressed as having a maximum number of substitutions (or terminal deletions) with respect to that reference sequence. For example, a sequence having at least Y % sequence identity (e.g., 90%) with SEQ ID NO:Z (e.g., 20 amino acids) may have up to X substitutions (e.g., 2) relative to SEQ ID NO:Z, and may therefore also be expressed as “having X (e.g., 2) or fewer substitutions relative to SEQ ID NO:Z.”

As used herein, “at least 90% sequence identity” refers to an oligonucleotide having at least 90%, at least 91%, at least 92%, at least 93%, at least 94%, at least 95%, at least 96%, at least 97%, at least 98%, at least 99%, or 100% sequence identity to the referenced sequence.

As used herein, the term “comprise” and linguistic variations thereof denote the presence of recited feature(s), element(s), method step(s), etc. without the exclusion of the presence of additional feature(s), element(s), method step(s), etc. Conversely, the term “consisting of” and linguistic variations thereof, denotes the presence of recited feature(s), element(s), method step(s), etc. and excludes any unrecited feature(s), element(s), method step(s), etc., except for ordinarily-associated impurities. The phrase “consisting essentially of” denotes the recited feature(s), element(s), method step(s), etc. and any additional feature(s), element(s), method step(s), etc. that do not materially affect the basic nature of the composition, system, or method. Many embodiments herein are described using open “comprising” language. Such embodiments encompass multiple closed “consisting of” and/or “consisting essentially of” embodiments, which may alternatively be claimed or described using such language. As used herein, comprising a certain sequence or a certain SEQ ID NO usually implies that at least one copy of said sequence is present in recited peptide or polynucleotide. However, two or more copies are also contemplated.

The term “contacting” as used herein refers to bring or put in contact, to be in or come into contact. The term “contact” as used herein refers to a state or condition of touching or of immediate or local proximity. Contacting a composition to a target destination, such as, but not limited to, an organ, tissue, cell, or tumor, may occur by any means of administration known to the skilled artisan.

The terms “non-naturally occurring,” “engineered,” and “synthetic” are used interchangeably and indicate the involvement of the hand of man. The terms, when referring to nucleic acid molecules or polypeptides mean that the nucleic acid molecule or the polypeptide is at least substantially free from at least one other component with which they are naturally associated in nature and as found in nature.

As used herein, a “nucleic acid” or a “nucleic acid sequence” refers to a polymer or oligomer of pyrimidine and/or purine bases, preferably cytosine, thymine, and uracil, and adenine and guanine, respectively (See Albert L. Lehninger, Principles of Biochemistry, at 793-800 (Worth Pub. 1982)). The present technology contemplates any deoxyribonucleotide, ribonucleotide, or peptide nucleic acid component, and any chemical variants thereof, such as methylated, hydroxymethylated, or glycosylated forms of these bases, and the like. The polymers or oligomers may be heterogenous or homogenous in composition and may be isolated from naturally occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states. In some embodiments, a nucleic acid or nucleic acid sequence comprises other kinds of nucleic acid structures such as, for instance, a DNA/RNA helix, peptide nucleic acid (PNA), morpholino nucleic acid (see, e.g., Braasch and Corey, Biochemistry, 41(14): 4503-4510 (2002)) and U.S. Pat. No. 5,034,506), locked nucleic acid (LNA; see Wahlestedt et al., Proc. Natl. Acad. Sci. U.S.A., 97:5633-5638 (2000)), cyclohexenyl nucleic acids (see Wang, J. Am. Chem. Soc., 122:8595-8602 (2000)), and/or a ribozyme. Hence, the term “nucleic acid” or “nucleic acid sequence” may also encompass a chain comprising non-natural nucleotides, modified nucleotides, and/or non-nucleotide building blocks that can exhibit the same function as natural nucleotides (e.g., “nucleotide analogs”); further, the term “nucleic acid sequence” as used herein refers to an oligonucleotide, nucleotide or polynucleotide, and fragments or portions thereof, and to DNA or RNA of genomic or synthetic origin, which may be single or double-stranded, and represent the sense or antisense strand. The terms “nucleic acid,” “polynucleotide,” “nucleotide sequence” are used interchangeably. They refer to a polymeric form of nucleotides of any length, either deoxyribonucleotides or ribonucleotides, or analogs thereof. The term “oligonucleotide,” or “oligos,” as used herein, generally refers to a short nucleic acid sequence comprising from about 2 to about 100 nucleotides (e.g., about 5, 10, 15, 20, 25, 30, 35, 40, 45, 50, 55, 60, 65, 70, 75, 80, 85, 90, 95, 99, or 100 nucleotides, or a range defined by any of the foregoing values) Any of the oligonucleotide sequences described herein may comprise, consist essentially of, or consist of a complement of any of the sequences disclosed herein.

The terms “protein,” “peptide,” and “polypeptide” are used interchangeably herein, and refer to a polymer of amino acid residues linked together by peptide bonds. The terms refer to a protein, peptide, or polypeptide of any size, structure, or function. Typically, a protein, peptide, or polypeptide will be at least three amino acids long. A protein, peptide, or polypeptide may refer to an individual protein or a collection of proteins. One or more of the amino acids in a protein, peptide, or polypeptide may be modified, for example, by the addition of a chemical entity such as a carbohydrate group, a hydroxyl group, a phosphate group, a farnesyl group, an isofarnesyl group, a fatty acid group, a linker for conjugation, functionalization, or other modification, etc. A protein, peptide, or polypeptide may also be a single molecule or may be a multi-molecular complex. A protein, peptide, or polypeptide may be just a fragment of a naturally occurring protein or peptide. A protein, peptide, or polypeptide may be naturally occurring, engineered, or synthetic, or any combination thereof. Any of the proteins provided herein may be produced by any method known in the art. For example, the proteins provided herein may be produced via recombinant protein expression and purification, which is especially suited for fusion proteins comprising a peptide linker. Methods for recombinant protein expression and purification are well known, and include those described by Green and Sambrook, Molecular Cloning: A Laboratory Manual (4th ed., Cold Spring Harbor Laboratory Press, Cold Spring Harbor, N.Y. (2012)), the entire contents of which are incorporated herein by reference.

As used herein, the terms “providing,” “administering,” and “introducing,” are used interchangeably herein and refer to the placement into a cell, organism, or subject by a method or route which results in at least partial localization to a desired site. For example, the compositions disclosed herein can be administered by any appropriate route which results in delivery to a desired location in the cell, organism, or subject.

A “subject” or “patient” are used interchangeably herein and refer to both human and nonhuman animals and may include, for example, animal strains or species used as “model systems” for research purposes, such a mouse model as described herein. Likewise, patient may include either adults or juveniles (e.g., children). Moreover, patient may mean any living organism, preferably a mammal (e.g., human or non-human) that may benefit from the administration of proteins, nucleic acids, or compositions contemplated herein. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish, and the like.

As used herein, “treat,” “treating,” “treatment,” and variations thereof refer to the clinical intervention made in response to a disease, disorder or physiological condition manifested by a patient or to which a patient may be susceptible. The aim of treatment includes the alleviation or prevention of symptoms, slowing or stopping the progression or worsening of a disease, disorder, or condition and/or the remission of the disease, disorder, or condition. A positive response to treatment may indicate a complete response to treatment, a partial response to treatment, or a stable disease state in the subject. A negative response to treatment may indicate disease progression in the subject. For example, treating cancer may include the management and care of the subject for combating and reducing one or more symptoms of the cancer. For example, treating cancer may reduce tumor burden (e.g., reduce the size of one or more tumors in the subject afflicted with cancer and/or reduce the overall number of tumors in the subject afflicted with cancer). Treating a cancer may reduce or completely eliminate the cancer (e.g., completely eliminate the tumor) in the subject.

As used herein, the term “immunotherapy” refers to any type of cancer treatment that helps the immune system fight cancer. For example, “immunotherapy” may include treatment with an immune checkpoint inhibitor, T-cell transfer therapy, monoclonal antibodies, treatment vaccines, and/or immune modulators. The cancer may be any cancer type.

As used herein, a method of predicting a response to treatment in a subject may include measuring a baseline level cancer associated ctDNA in the subject. The baseline level of said ctDNA may be measured prior to any treatment in the subject. Alternatively, the baseline level of said ctDNA may be measured following one or more treatment sessions in the subject.

The method may further include measuring a follow-on level of cancer associated ctDNA following one or more treatment sessions in the subject. For example, a baseline level of said ctDNA may be measured prior to any therapy and a follow-on level may be measured following a therapy cycle. Alternatively, a baseline level of said ctDNA may be measured after a first therapy cycle and a follow-on level may be measured following a second therapy cycle.

The baseline level and/or follow on level of said ctDNA may be measured using a suitable method described herein. For example, measuring a baseline level and measuring a follow-on level may include obtaining a sample comprising ctDNA from the subject.

The cell free DNA may be isolated from plasma obtained from the subject. For example, plasma may be obtained and cell free DNA may be isolated using any suitable method. In some embodiments, cell free DNA is isolated using a commercially available kit.

In some embodiments, the method further involves predicting response to therapy in the subject based upon HPV (e.g., HPV16) ctDNA levels. In some embodiments, the method comprises predicting a positive response to treatment in the subject when a change from the baseline level to the follow-on level is below a threshold value. In some embodiments, the method comprises predicting a negative response to treatment in the subject when a change from the baseline level to the follow-on level is above a threshold value. In some embodiments, the threshold value is 50%. In some embodiments, the threshold value is 60%. For example, the method may comprise predicting a positive response to treatment when there is less than a 60% increase in HPV ctDNA levels from the baseline level to the follow-on level. Alternatively, the method may comprise predicting a negative response to treatment when there is a 60% or higher increase in HPV ctDNA levels from the baseline level to the follow-on level. A positive response to treatment may indicate a complete response to treatment, a partial response to treatment, or a stable disease state in the subject. For example, a positive response may indicate no metastasis, no increase in tumor size, and/or no increase in total number of tumors observed in the subject. A negative response may indicate progression of the disease, such as increased total number of tumors and/or increased tumor size.

An “amplicon” refers to a nucleic acid fragment formed as a product of natural or artificial amplification events or techniques. For example, an amplicon can be produced by PCR.

As used herein, “amplify,” “amplifying,” “amplification,” and variations thereof refer to method of increasing the number of copies of a target sequence, if present in a sample. In some embodiments, the method further comprises detecting a signal from a detectable label, which is indicative of the presence of the target sequence, if present in the sample. For example, amplification may be performed by polymerase chain reaction (PCR).

As used herein, “polymerase chain reaction” (PCR) refers to amplification and “quantitative PCR” (qPCR) refers to a method of quantifying the number of copies of the amplified sequence. For example, amplification and quantification of a target sequence may be performed using a digital PCR technique, such as a dPCR technique selected from droplet digital PCR (ddPCR), BEAMing (beads, emulsion, amplification, and magnetic), and microfluidic chips.

As used herein, “digital PCR” refers to an assay that provides an end-point measurement that provides the ability to quantify nucleic acids without the use of standard curves, as is used in real-time PCR. In a typical digital PCR experiment, the sample is randomly distributed into discrete partitions, such that some contain no nucleic acid template and others contain one or more template copies. The partitions are amplified to the terminal plateau phase of PCR (or end-point) and then read to determine the fraction of positive partitions. If the partitions are of uniform volume, the number of target DNA molecules present may be calculated from the fraction of positive end-point reactions using, for example, Poisson statistics, according to the following equation:

λ=−1n(1−p) (1)
wherein λ is the average number of target DNA molecules per replicate reaction and p is the fraction of positive end-point reactions. From k, together with the volume of each replicate PCR and the total number of replicates analyzed, an estimate of the absolute target DNA concentration is calculated. Digital PCR includes a variety of formats, including droplet digital PCR, BEAMing (beads, emulsion, amplification, and magnetic), and microfluidic chips.

As used herein, “droplet digital PCR” (ddPCR) refers to a digital PCR assay that measures absolute quantities by counting nucleic acid molecules encapsulated in discrete, volumetrically defined, water-in-oil droplet partitions that support PCR amplification (Hinson et al., 2011, Anal. Chem. 83:8604-8610; Pinheiro et al., 2012, Anal. Chem. 84:1003-1011). A single ddPCR reaction may be comprised of at least 20,000 partitioned droplets per well. A “droplet” or “water-in-oil droplet” refers to an individual partition of the droplet digital PCR assay. A droplet supports PCR amplification of template molecule(s) using homogenous assay chemistries and workflows similar to those widely used for real-time PCR applications (Hinson et al., 2011, Anal. Chem. 83:8604-8610; Pinheiro et al., 2012, Anal. Chem. 84:1003-1011).

Droplet digital PCR may be performed using any platform that performs a digital PCR assay that measures absolute quantities by counting nucleic acid molecules encapsulated in discrete, volumetrically defined, water-in-oil droplet partitions that support PCR amplification. The strategy for droplet digital PCR may be summarized as follows: a sample is diluted and partitioned into thousands to millions of separate reaction chambers (water-in-oil droplets) so that each contains one or no copies of the nucleic acid molecule of interest. The number of “positive” droplets detected, which contain the target amplicon (e.g., nucleic acid molecule of interest), versus the number of “negative” droplets, which do not contain the target amplicon (e.g., nucleic acid molecule of interest), may be used to determine the number of copies of the nucleic acid molecule of interest that were in the original sample. Examples of droplet digital PCR systems include the QX100™ Droplet Digital PCR System by Bio-Rad, which partitions samples containing nucleic acid template into 20,000 nanoliter-sized droplets; the QX200™ Droplet Digital PCR System by Bio-Rad; and the RainDrop™ digital PCR system by RainDance, which partitions samples containing nucleic acid template into 1,000,000 to 10,000,000 picoliter-sized droplets.

As used herein, “detectable label” refers to a fluorescent label affixed to an oligonucleotide. For example, suitable fluorescent labels include, FAM (5- or 6-carboxyfluorescein), VIC, NED, Fluorescein, FITC, IRD-700/800, CY3, CY5, CY3.5, CY5.5, HEX, TET, TAMRA, JOE, ROX, BODIPY TMR, Oregon Green, Rhodamine Green, Rhodamine Red, Texas Red, Yakima Yellow, Alexa Fluor PET, Biosearch Blue™, Marina Blue®, Bothell Blue®, Alexa Fluor®, 350 FAM™, SYBR® Green 1, Fluorescein, EvaGreen™, Alexa Fluor® 488 JOE™, VIC™ HEX™ TET™, CAL Fluor® Gold 540, Yakima Yellow®, ROX™, CAL Fluor® Red 610, Cy3.5™, Texas Red®, Alexa Fluor® 0.568 Cy5™, Quasar™ 670, LightCycler Red640®, Alexa Fluor 633 Quasar™ 705, LightCycler Red705®, Alexa Fluor® 680, SYTO® 9, LC Green®, LC Green® Plus+, and EvaGreen™ Further, a detectable label is used to detect the presence of a specific molecule in a sample wherein detection of the signal indicates the presence of the target sequence. Quantification of the target sequence may be performed, for example, by determining the number of target molecules present. The number of target molecules present may be calculated from the fraction of positive end-point reactions using Poisson statistics, as described above. The methods of detecting, amplifying, and/or quantifying the target sequence may be used in a variety of diagnostic or predictive methods. For example, the methods may be used to determine whether a subject has cancer and/or for predicting a subject's response to therapy in a cancer.

### 2. Compositions

Embodiments of the present disclosure include compositions comprising one or more oligonucleotides (e.g., primer/probe sets suitable for use in a multi-probe HPV ctDNA assay).

In some embodiments, the present disclosure provides the design, development, and validation of a multi-probe HPV composition that offers a precise assay test for detection of HPV-related cancers (e.g., back of the throat, cervical, anus, vulva, penis, vagina, and oropharynx (e.g., HPV+ OPSCC)).

In some embodiments, the one or more oligonucleotides (e.g., primer/probe sets) are designed to detect, amplify, and/or quantify one or more analytes (e.g., nucleic acids (e.g., prognostic or predictive DNA (e.g., viral nucleic acid and/or ctDNA (e.g., HPV-related cancer (e.g., back of the throat, cervical, anus, vulva, penis, vagina, HPV+ OPSCC) DNA)))) from various samples.

In some embodiments, detection and/or amplification of one or more analytes is by target amplification (e.g., polymerase chain reaction (PCR), reverse transcriptase-PCR (RT-PCR), strand displacement amplification, transcription amplification), signal amplification (e.g., branched DNA assays, hybrid capture), probe amplification (e.g., ligase chain reaction, cleavase-invader, cycling probes), or postamplification analysis (e.g., sequencing of the amplified product, microarray analysis, and melting curve analysis, as is done in real-time PCR).

In some embodiments, quantification of one or more analytes is by absorbance (e.g., UV spectroscopy), by fluorescence (e.g., use of fluorescent dyes (e.g., specific dyes used to stain only specific types of nucleic acid (e.g., ssDNA, miRNA, dsDNA, and/or RNA))), by electrophoresis (e.g., if the DNA to be quantified is a plasmid, then a fluorescent dye (e.g., ethidium bromide or SYBR Green) is added to the gel or the sample, followed by electrophoresis of the sample parallel to a DNA ladder), by PCR (e.g., quantitative real-time PCR (qPCR), digital PCR or Droplet Digital PCR (ddPCR)).

In some embodiments, the one or more analytes is the one or more analytes are HPV-related cancer (e.g., back of the throat, cervical, anus, vulva, penis, vagina, and oropharynx (e.g., HPV+ OPSCC) DNA.

In some embodiments, the oligonucleotides (e.g., primer/probe sets) are designed to detect, amplify and/or quantify one or more target regions of a HPV type (e.g., HPV16, HPV18, HPV31, HPV33, HPV35, HPV39, HPV45, HPV51, HPV52, HPV56, HPV58, HPV59, HPV66, HPV68).

In some embodiments, the oligonucleotides (e.g., primer/probe sets) comprise: SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36; SEQ ID NO: 52; SEQ ID NO: 53; SEQ ID NO: 54; SEQ ID NO: 56.

In some embodiments, the oligonucleotides (e.g., primer/probe sets) comprise: SEQ ID NOs: 1-27 and SEQ ID NOs: 37-51.

In some embodiments, at least a triad of the oligonucleotides (e.g., primer/probe sets) comprise: Set 1: SEQ ID NO: 1, SEQ ID NO: 2, SEQ ID NO: 3; Set 2: SEQ ID NO: 4, SEQ ID NO: 5, SEQ ID NO: 6; Set 3: SEQ ID NO: 7, SEQ ID NO: 8, SEQ ID NO: 9; Set 4: SEQ ID NO: 10, SEQ ID NO: 11, SEQ ID NO: 12; Set 5: SEQ ID NO: 13, SEQ ID NO: 14, SEQ ID NO: 15; Set 6: SEQ ID NO: 16, SEQ ID NO: 17, SEQ ID NO: 18; Set 7: SEQ ID NO: 19, SEQ ID NO: 20, SEQ ID NO: 21; Set 8: SEQ ID NO: 22, SEQ ID NO: 23, SEQ ID NO: 24; Set 9: SEQ ID NO: 25, SEQ ID NO: 26, SEQ ID NO: 27; Set 10: SEQ ID NO: 37, SEQ ID NO: 38, SEQ ID NO: 39; Set 11: SEQ ID NO: 40, SEQ ID NO: 41, SEQ ID NO: 42; Set 12: SEQ ID NO: 43, SEQ ID NO: 44, SEQ ID NO: 45; Set 13: SEQ ID NO: 46, SEQ ID NO: 47, SEQ ID NO: 48; Set 14: SEQ ID NO: 49, SEQ ID NO: 50, SEQ ID NO: 51.

In some embodiments, the oligonucleotides (e.g., primer/probe sets) comprise: a forward primer having at least 90% sequence identity to any one or more of: SEQ ID NOs: 1, 4, 7, 10, 13, 16, 19, 22, 25, 37, 40, 43, 46, 49; a reverse primer having at least 90% sequence identity to any one or more of: SEQ ID NOs: 2, 5, 8, 11, 14, 17, 20, 23, 26, 39, 42, 45, 48, 51; a probe comprising a detectable label, wherein the forward primer and the reverse primer anneal to a target sequence.

In some embodiments, the probe has at least 90% sequence identity to any one or more of: SEQ ID NOs: 3, 6, 9, 12, 15, 18, 21, 24, 27, 38, 41, 44. 47, 50.

In some embodiments, a first set of oligonucleotides comprises SEQ ID NOs: 1-15 or SEQ ID NOs: 37-45.

In some embodiments, a second set of oligonucleotides comprises SEQ ID NOs: 16-27 or SEQ ID NOs: 46-51.

In some embodiments, a probe used with any of the oligonucleotides (e.g., primer/probe sets) comprises a sequence that hybridizes to a target sequence between or overlapping the primer sequences.

In some embodiments, the oligonucleotides (e.g., primer/probe sets) does not have greater than an eight-base pair stretch of bases annealing as a homodimer, hairpin, or heterodimer.

In some embodiments, a first set of oligonucleotides comprises a first detectable label and a second set of oligonucleotides comprises a second detectable label.

In some embodiments, a second set of oligonucleotides, comprising a second detectable label, targets a viral E6/E7 junction.

In some embodiments, the viral E6/E7 junction is highly represented in HPV16 ctDNA.

In some embodiments, a first labeled set of oligonucleotides passes a two-fold criterion and does not alter the background in a second label channel when compared to a single probe set of oligonucleotides.

In some embodiments, a second set of oligonucleotides, comprising a second detectable label, is selected from the group with SEQ ID Nos: 2, 5, 8, 11, 14, 17, 20, 23, 26, 39, 42, 45, 48, 51.

In some embodiments, a first set of oligonucleotides, comprising a first detectable label, is selected from the group with SEQ ID Nos: SEQ ID NOs: 1, 4, 7, 10, 13, 16, 19, 22, 25, 37, 40, 43, 46, 49.

In some embodiments, a second set of oligonucleotides, comprising a second detectable label, targets a viral E6/E7 junction.

### 3. Methods

Embodiments of the present disclosure include methods of detecting, amplifying, and/or quantifying one or more analytes (e.g., nucleic acids (e.g., prognostic or predictive DNA (e.g., viral nucleic acid and/or ctDNA (e.g., HPV-related cancer (e.g., back of the throat, cervical, anus, vulva, penis, vagina, HPV+ OPSCC) DNA)))) from various samples.

In some embodiments, any number of different detecting, amplifying, and/or quantifying methodologies may be used.

In some embodiments, the detecting, amplifying, and/or quantifying occurs prior to the incidence of disease (e.g., prior to awareness of a disease state in a subject (e.g., prior to the appearance of CT scan-detectable disease)).

In some embodiments, the methods are designed to detect, amplify and/or quantify one or more target regions of a HPV type (e.g., HPV16, HPV18, HPV31, HPV33, HPV35, HPV39, HPV45, HPV51, HPV52, HPV56, HPV58, HPV59, HPV66, HPV68).

In some embodiments, the detecting, amplifying, and/or quantifying is by an analytical method which uses a labeled antigen.

In some embodiments, the detecting, amplifying, and/or quantifying is by contacting a sample comprising one or more analytes (e.g., nucleic acids (e.g., prognostic or predictive DNA (e.g., viral nucleic acid and/or ctDNA (e.g., HPV-related cancer HPV-related cancer (e.g., back of the throat, cervical, anus, vulva, penis, vagina, HPV+ OPSCC) DNA)))) with one or more oligonucleotides.

In some embodiments, the one or more oligonucleotides comprise a forward primer, a reverse primer, and a probe.

In some embodiments, detecting, amplifying, and/or quantifying comprises isolating, purifying, and/or concentrating analytes (e.g., nucleic acids (e.g., prognostic or predictive DNA (e.g., viral nucleic acid and/or ctDNA (e.g., HPV-related cancer (e.g., back of the throat, cervical, anus, vulva, penis, vagina, HPV+ OPSCC) DNA)))) from various samples.

In some embodiments, purifying is used to concentrate one or more analytes (e.g., nucleic acids (e.g., prognostic or predictive DNA (e.g., viral nucleic acid and/or ctDNA (e.g., HPV-related cancer (e.g., back of the throat, cervical, anus, vulva, penis, vagina, HPV+OPSCC) DNA)))) from various samples so that they are directly available for downstream applications such as subsequent analysis methods (e.g., in order to amplify, identify, quantify and/or detect the presence or absence of a certain target nucleic acid within the sample).

In some embodiments, the one or more analytes (e.g., nucleic acids (e.g., prognostic or predictive DNA (e.g., viral nucleic acid and/or ctDNA))) in the sample is a HPV type (e.g., HPV16, HPV18, HPV31, HPV33, HPV35, HPV39, HPV45, HPV51, HPV52, HPV56, HPV58, HPV59, HPV66, HPV68).

In some embodiments, the one or more analytes (e.g., nucleic acid (e.g., prognostic or predictive DNA (e.g., viral nucleic acid and/or ctDNA (e.g., HPV-related cancer (e.g., back of the throat, cervical, anus, vulva, penis, vagina, HPV+ OPSCC) DNA)))) is detected and/or quantified by contacting a sample with one or more oligonucleotides (e.g., primer/probe sets).

In some embodiments, the one or more oligonucleotides (e.g., primer/probe sets) comprise a forward primer, a reverse primer, and a probe.

In some embodiments, the sample is amniotic fluid, ascites, bile, breast milk, breast milk colostrum, bronchoalveolar lavage fluid, cerebrospinal fluid, dialysate, eye aqueous humor, eye vitreous humor, feces, paracentesis, pericardial fluid, peritoneal, blood plasma, pleural, semen, blood serum, synovial fluid, tears, thoracentesis, blood, saliva, gargle, or urine, is derived from any such sample, although any other sample type may be used.

In some embodiments, contacting a sample comprises: providing one or more oligonucleotides (e.g., primer/probe sets); fractionating (e.g., fractionating by emulsification) a plurality of analytes (e.g., nucleic acids (e.g., prognostic or predictive DNA (e.g., viral nucleic acid and/or ctDNA (e.g., HPV-related cancer (e.g., back of the throat, cervical, anus, vulva, penis, vagina, and HPV+ OPSCC) DNA)))) from the sample into droplets.

In some embodiments, droplets are at a concentration wherein only 0 or 1 molecule of the analyte (e.g., nucleic acids (e.g., prognostic or predictive DNA (e.g., viral nucleic acid and/or ctDNA (e.g., HPV-related cancer (e.g., back of the throat, cervical, anus, vulva, penis, vagina, and HPV+ OPSCC) DNA)))) is present in each droplet; amplifying the analyte in each droplet with one or more oligonucleotides (e.g., primer/probe sets) to produce amplicon signals; and detecting in each droplet any amplicon signals.

In some embodiments, the one or more analytes (e.g., nucleic acids (e.g., prognostic or predictive DNA (e.g., viral nucleic acid and/or ctDNA (e.g., HPV-related cancer (e.g., back of the throat, cervical, anus, vulva, penis, vagina, and HPV+ OPSCC) DNA)))) is fractionated into micro-droplets by emulsification.

In some embodiments, the one or more analytes (e.g., nucleic acids (e.g., prognostic or predictive DNA (e.g., viral nucleic acid and/or ctDNA (e.g., HPV-related cancer (e.g., back of the throat, cervical, anus, vulva, penis, vagina, and HPV+ OPSCC) DNA)))) is amplified using a detection and/or amplification method.

In some embodiments, detection and/or amplification of one or more analytes is by target amplification (e.g., polymerase chain reaction (PCR), reverse transcriptase-PCR (RT-PCR), strand displacement amplification, transcription amplification), signal amplification (e.g., branched DNA assays, hybrid capture), probe amplification (e.g., ligase chain reaction, cleavase-invader, cycling probes), or postamplification analysis (e.g., sequencing of the amplified product, microarray analysis, and melting curve analysis, as is done in real-time PCR).

In some embodiments, quantification of one or more analytes is by absorbance (e.g., UV spectroscopy), by fluorescence (e.g., use of fluorescent dyes (e.g., specific dyes used to stain only specific types of nucleic acid (e.g., ssDNA, miRNA, dsDNA, and/or RNA))), by electrophoresis (e.g., if the DNA to be quantified is a plasmid, then a fluorescent dye (e.g., ethidium bromide or SYBR Green) is added to the gel or the sample, followed by electrophoresis of the sample parallel to a DNA ladder), by PCR (e.g., quantitative real-time PCR (qPCR), digital PCR or Droplet Digital PCR (ddPCR)). In some embodiments, contacting comprises conducting a quantitative PCR (qPCR) assay. In some embodiments, a qPCR assay comprises a digital PCR assay. In some embodiments, a digital PCR assay comprises a droplet digital PCR (ddPCR) assay.

In some embodiments, the sample comprises a target nucleic acid (e.g., HPV target nucleic acid). In some embodiments, the one or more oligonucleotides hybridize to the target nucleic acid.

In some embodiments, the detection, amplification, and/or quantification method (e.g., the assay) has a limit of detection (LOD) of <1 genome equivalent of target nucleic acid (e.g., HPV target nucleic acid).

In some embodiments, the target nucleic acid (e.g., HPV nucleic acid) or a product derived therefrom (e.g., an amplicon) is sequenced.

In some embodiments, the sequencing technique is a next generation sequencing technique. The term “next generation sequencing” refers to highly parallelized methods of performing nucleic acid sequencing and comprises the sequencing-by-synthesis or sequencing-by-ligation platforms (e.g., employed by Illumina, Life Technologies, Pacific Biosciences and Roche, etc.). Next generation sequencing methods may also include, but not be limited to, nanopore sequencing methods such as offered by Oxford Nanopore or electronic detection-based methods such as the Ion Torrent technology commercialized by Life Technologies.

In some embodiments, one or more oligonucleotides (e.g., primers) described herein further comprises an additional sequence (e.g., barcode, adapter, etc.) that finds use in sequencing library preparation, sequencing, and analysis.

In some embodiments, the sequencing technique is a next generation sequencing technique. The term “next generation sequencing” refers to highly parallelized methods of performing nucleic acid sequencing and comprises the sequencing-by-synthesis or sequencing-by-ligation platforms (e.g., employed by Illumina, Life Technologies, Pacific Biosciences and Roche, etc.). Next generation sequencing methods may also include, but not be limited to, nanopore sequencing methods such as offered by Oxford Nanopore or electronic detection-based methods such as the Ion Torrent technology commercialized by Life Technologies. In some embodiments, one or more of the primers described herein further comprises an additional sequence (e.g., barcode, adapter, etc.) that finds use in sequencing library preparation, sequencing, and analysis

Suitable nucleic acid sequencing techniques include, but are not limited to, sequencing by synthesis (see e.g., Meyer and Kircher, “Illumina sequencing library preparation for highly multiplexed target capture and sequencing,” Cold Spring Harbor Protocols 2010 (6)); single-molecule real-time sequencing (see e.g., Levene et al., “Zero-Mode Waveguides for Single-Molecule Analysis at High Concentrations,” Science. 299 (5607): 682-6 (2003)); ion semiconductor sequencing (see e.g., Rusk, “Torrents of sequence,” Nat. Methods 8, 44 (2011)); pyrosequencing (see e.g., Wicker et al., “454 sequencing put to the test using the complex genome of barley,” BMC Genomics, 7:275, 2006); sequencing by ligation (SOLID sequencing) (see e.g., Margulies et al., “Genome sequencing in microfabricated high-density picolitre reactors,” Nature, 437:376-80 (2005)); nanopore sequencing (see e.g., Goodwin et al., “Oxford Nanopore sequencing, hybrid error correction, and de novo assembly of a eukaryotic genome,” Genome Res., 25 (11): 1750-6 (2015)); chain termination sequencing (Sanger sequencing) (see e.g., Sanger et al., “DNA sequencing with chain-terminating inhibitors, “Proceedings of the National Academy of Sciences of the United States of America, 74 (12): 5463-5467 (1977)); and sequencing with mass spectrometry (see e.g., Edwards et al., “Mass-spectrometry DNA sequencing,” Mutation Research, 573 (1-2): 3-12 (2005)).

Embodiments of the present disclosure also include methods of detecting one or more (e.g., nucleic acids (e.g., prognostic or predictive DNA (e.g., viral nucleic acid and/or ctDNA (e.g., HPV-related cancer (e.g., back of the throat, cervical, anus, vulva, penis, vagina, and HPV+ OPSCC) DNA)))) in a sample provided by a subject and treating the subject if HPV-related cancer DNA is detected in the sample.

In some embodiments, treating a subject means treating with cancer surveillance (e.g., closely watching a subject's condition, but not administering a treatment unless there are changes in test results that show the condition is getting worse), treating with a cancer therapeutic, or treating with another intervention.

In some embodiments, treating a subject means treating with an imaging technique (e.g., computed tomography (CT) scan, magnetic resonance imaging (MRI) scan, x-rays and other radiographic tests, mammography, nuclear medicine scans (e.g., bone scans, PET scans, thyroid scans, MUGA scans, gallium scans), and ultrasound), a clinical examination, excision treatment, thermal ablation (e.g., cryoablation (e.g., PVP laser surgery, cryosurgery, or cryotherapy), radiofrequency, ablation), radiotherapy (e.g., intraoperative radiotherapy, stereotactic surgery, proton therapy, 3D conformal radiation therapy, brachytherapy, protons, total body irradiation, electrons, internal radiation, short-course radiation therapy, volumetric modulated arc therapy, linear accelerator, photons), chemotherapy (e.g., alkylating antineoplastic agent, topical, peritoneal chemotherapy, cyclophosphamide, etoposide, doxorubicin, irinotecan, antibiotics, topoisomerase inhibitors, plant alkaloids, carboplatin, daunorubicin, oral, gemcitabine), hormone therapy (e.g., hormone therapy, hormonal therapy, or endocrine therapy) and/or salvage therapy (e.g., a second therapy after a first therapy is ineffective or the patient is intolerant).

In some embodiments, treating a subject means treating with an approach such as palliative care, participation in a clinical trial, precision or personalized medicine, off-label drugs, biosimilar drugs, tumor-agnostic drugs, and/or pharmacogenomic testing.

In some embodiments, the treatment is provided prior to the incidence of disease (e.g., prior to awareness of a disease state in a subject (e.g., prior to the appearance of CT scan-detectable disease)).

Embodiments of the present disclosure also include methods of detecting HPV-related cancer (e.g., back of the throat, cervical, anus, vulva, penis, vagina, HPV+ OPSCC) DNA in a sample provided by a subject and treating the subject with an HPV vaccine (e.g., 9-valent HPV vaccine (e.g., Gardasil 9, 9vHPV), quadrivalent HPV vaccine (e.g., Gardasil, 4vHPV), and bivalent HPV vaccine (e.g., Cervarix, 2vHPV)) if HPV is not detected in the sample.

### 4. Kits

Embodiments of the present disclosure include kits comprising one or more oligonucleotides (e.g., primer/probe sets), as described herein.

In some embodiments, the kits may further comprise reagents necessary, useful, or sufficient to purify, isolate, detect and/or quantify one or more analytes (e.g., nucleic acids (e.g., prognostic or predictive DNA (e.g., viral nucleic acid and/or ctDNA (e.g., HPV-related cancer (e.g., back of the throat, cervical, anus, vulva, penis, vagina, and HPV+ OPSCC) DNA)))). For example, the kits may further comprise amplification reagents, including buffers and enzymes.

In some embodiments, the kit may further comprise control samples, if needed or desired.

In some embodiments, the kit may comprise solid surfaces (e.g., beads) comprising capture reagents (e.g., oligonucleotides) specific for one or more (e.g., nucleic acids (e.g., prognostic or predictive DNA (e.g., viral nucleic acid and/or ctDNA (e.g., HPV-related cancer (e.g., back of the throat, cervical, anus, vulva, penis, vagina, and HPV+ OPSCC) DNA)))).

In some embodiments, the kit may further comprise containers for holding or storing a sample, reagents, or reaction mixtures (e.g., a container or cartridge for a plasma sample, a container for a sample (e.g., a ctDNA sample), etc.).

In some embodiments, the kit may further comprise one or more instruments for assisting with obtaining or manipulating a test sample (e.g., a syringe). Where appropriate, the kit may comprise reaction vessels, mixing vessels, and other components that facilitate the preparation of reagents (e.g., a container for mixing reagents for analysis).

In some embodiments, the kit may further comprise instructions for use of the kit. Instructions included in kits can be affixed to packaging material or can be included as a package insert or can be viewed or downloaded from a particular website that is recited as part of the kit packaging or inserted materials. While the instructions are typically written or printed materials, they are not limited to such. Any medium capable of storing such instructions and communicating them to an end user is contemplated by this disclosure. Such media include, but are not limited to, electronic storage media (e.g., magnetic discs, tapes, cartridges, chips), optical media (e.g., CD ROM), and the like. As used herein, the term “instructions” can include the address of an Internet site that provides the instructions.

### 5. Sequences

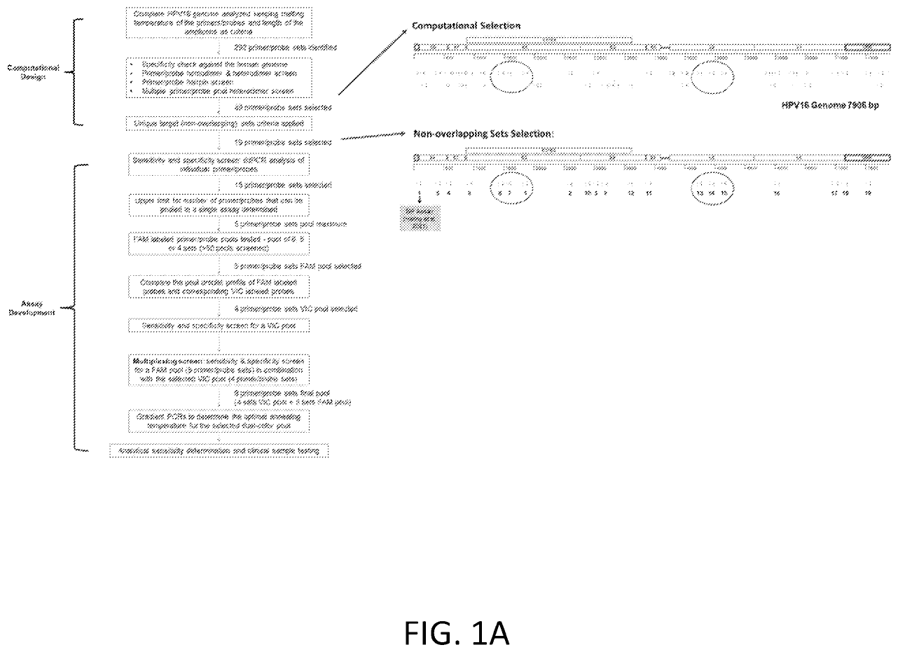

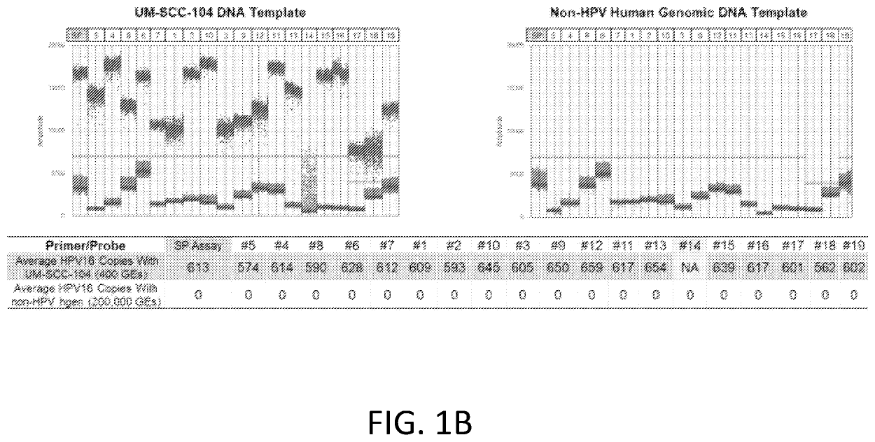

TABLE 1
Sequences of the Primer and Probe Sets That Were Experimentally Tested
in Addition to Sequences of the Primer and Probe Sets Selected for the
CHAMP-16 Assay Pool.

| | | | | | | Experi- |
|---|---|---|---|---|---|---|
| | | | | | | mentally |
| | | | | | | Tested in a |
| Primer/ | | SEQ | | | | Singleplex |
| Probe | | ID | | Oligonucleotide | | Assay |
| Set | Name | NO: | Sequence | Group | Length | (Fig. 1B) |
| 1 | sequence_232 | 61 | GCAGCAGCATTATATTGGTAT | Forward Primer | 21 | Yes |
| 1 | sequence_233 | 62 | TATTAGTGAAGTGTATGGAGAC | Probe | 25 | Yes |
| | | | ACG | | | |
| 1 | sequence_234 | 63 | TTTGTCTTTGTATCCATTCTGG | Reverse Primer | 22 | Yes |
| 3 | sequence_370 | 64 | ATTGAACTGCAACTAACG | Forward Primer | 18 | Yes |
| 3 | sequence_371 | 65 | AGTGGACATTACAAGACGTT | Probe | 20 | Yes |
| 3 | sequence_372 | 66 | GTTAAATACACTTCAAGGCT | Reverse Primer | 20 | Yes |
| 8 | sequence_208 | 67 | TCCTGCAGGTACCAATG | Forward Primer | 17 | Yes |
| 8 | sequence_209 | 68 | CATTACATCCCGTACCCTCTTC | Probe | 22 | Yes |
| 8 | sequence_210 | 69 | GTCATCTGATATAGCATCCC | Reverse Primer | 20 | Yes |
| 9 | sequence_301 | 70 | TTGATGGAGACATATGCAATAC | Forward Primer | 22 | Yes |
| 9 | sequence_302 | 71 | TGTGAAGAAGCATCAGTAACTG | Probe | 22 | Yes |
| 9 | sequence_303 | 72 | CAACTTGACCCTCTACCA | Reverse Primer | 18 | Yes |
| 10 | sequence_349 | 73 | CATATAGACTATTGGAAACACATG | Forward Primer | 25 | Yes |
| | | | C | | | |
| 10 | sequence_350 | 74 | CTCTGGCCTTGTAATAAATAGCAC | Probe | 24 | Yes |
| 10 | sequence_351 | 75 | CTGGTGGTTAATATGTTTAAATCCC | Reverse Primer | 25 | Yes |
| 11 | sequence_412 | 76 | GACAAATCTTGATACTGCATCC | Forward Primer | 22 | Yes |
| 11 | sequence_413 | 77 | CAACATTACTGGCGTGCTTT | Probe | 20 | Yes |
| 11 | sequence_414 | 78 | GGACGTATTAATAGGCAGACA | Reverse Primer | 21 | Yes |
| 12 | sequence_475 | 79 | CTGCATTTAACAGCTCAC | Forward Primer | 18 | Yes |
| 12 | sequence_476 | 80 | ACAAAGGACGGATTAACTGT | Probe | 20 | Yes |
| 12 | sequence_477 | 81 | TAAATGTACTATGGGTGTAG | Reverse Primer | 20 | Yes |
| 14 | sequence_562 | 82 | GGCACGCCTAGGATTAT | Forward Primer | 17 | Yes |
| 14 | sequence_563 | 83 | CACAACAGGTTAAAGTTGTAGACC | Probe | 24 | Yes |
| 14 | sequence_564 | 84 | AGTTTAGTGGGAGTGGTTAC | Reverse Primer | 20 | Yes |
| 17 | sequence_790 | 85 | GCACCTAAAGAAGATCCC | Forward Primer | 18 | Yes |
| 17 | sequence_791 | 86 | TTTCTGCAGACCTAGATCAG | Probe | 20 | Yes |
| 17 | sequence_792 | 87 | TTTGCGTCCTAAAGGAAA | Reverse Primer | 18 | Yes |
| 18 | sequence_796 | 88 | CCCACCACCTCATCTAC | Forward Primer | 17 | Yes |
| 18 | sequence_797 | 89 | CTCTACAACTGCTAAACGCAAA | Probe | 22 | Yes |
| 18 | sequence_798 | 90 | ACACAACAAACAACACT | Reverse Primer | 17 | Yes |

TABLE 2
Sequences of the Primer and Probe Sets That Passed Computational Screen.

| Primer/ | | SEQ | | Oligo- | |
|---|---|---|---|---|---|
| Probe | | ID | | nucleotide | |
| Set | Name | NO: | Sequence | Group | Length |
| 20 | sequence_004 | 91 | CCAGAAAGTTACCACAGTTATG | Forward Primer | 22 |
| 20 | sequence_005 | 92 | CACAGAGCTGCAAACAACTATAC | Probe | 23 |
| 20 | sequence_006 | 93 | CTGTTGCTTGCAGTACAC | Reverse Primer | 18 |
| 21 | sequence_028 | 94 | CTCTACGCTTCGGTTGT | Forward Primer | 17 |
| 21 | sequence_029 | 95 | CGTAGACATTCGTACTTTGGAAGACC | Probe | 26 |
| 21 | sequence_030 | 96 | TCCTAGTGTGCCCATTAAC | Reverse Primer | 19 |
| 22 | sequence_046 | 97 | CACGTAGACATTCGTACTTTG | Forward Primer | 21 |
| 22 | sequence_047 | 98 | CAATTCCTAGTGTGCCCATTAAC | Probe | 23 |
| 22 | sequence_048 | 99 | GTAGATTATGGTTTCTGAGAACAG | Reverse Primer | 24 |
| 23 | sequence_052 | 100 | GTATGTCTTGTTGCAGATCAT | Forward Primer | 21 |
| 23 | sequence_053 | 101 | AGAACACGTAGAGAAACCCAG | Probe | 21 |
| 23 | sequence_054 | 102 | TGTAGGTGTATCTCCATGC | Reverse Primer | 19 |
| 24 | sequence_076 | 103 | GCTCAGAGGAGGAGGAT | Forward Primer | 17 |
| 24 | sequence_077 | 104 | CGGACAGAGCCCATTACAATA | Probe | 21 |
| 24 | sequence_078 | 105 | GAGTCACACTTGCAACAAA | Reverse Primer | 19 |
| 25 | sequence_079 | 106 | GAGCAATTAAATGACAGCTCAG | Forward Primer | 22 |
| 25 | sequence_080 | 107 | CTGGACCATCTATTTCATCCTCC | Probe | 23 |
| 25 | sequence_081 | 108 | GTCCGGTTCTGCTTGTC | Reverse Primer | 17 |
| 26 | sequence_115 | 109 | CACGTAGACATTCGTACTTTG | Forward Primer | 21 |
| 26 | sequence_116 | 110 | CAATTCCTAGTGTGCCCATTAAC | Probe | 23 |
| 26 | sequence_117 | 111 | GCCATGGTAGATTATGGTTTC | Reverse Primer | 21 |
| 27 | sequence_157 | 112 | GAGATGCAGTACAGGTTCTAA | Forward Primer | 21 |
| 27 | sequence_158 | 113 | ACGAAAGTATTTGGGTAGTCCA | Probe | 22 |
| 27 | sequence_159 | 114 | GTCTACACATCCACTAATATCACTA | Reverse Primer | 25 |
| 28 | sequence_178 | 115 | CATTCGTACTTTGGAAGACC | Forward Primer | 20 |
| 28 | sequence_179 | 116 | CAATTCCTAGTGTGCCCATTAAC | Probe | 23 |
| 28 | sequence_180 | 117 | GTAGATTATGGTTTCTGAGAACAG | Reverse Primer | 24 |
| 29 | sequence_205 | 118 | CAACATAGAGATGCAGTACAGG | Forward Primer | 22 |
| 29 | sequence_206 | 119 | CGAAAGTATTTGGGTAGTCCACTTA | Probe | 25 |
| 29 | sequence_207 | 120 | TTGTCTACACATCCACTAAT | Reverse Primer | 20 |
| 30 | sequence_253 | 121 | TGCAAGTGCCTTTCTAA | Forward Primer | 17 |
| 30 | sequence_254 | 122 | AGGATTGTGCAACAATGTGT | Probe | 20 |
| 30 | sequence_255 | 123 | TCTGCTCGTTTATAATGTCT | Reverse Primer | 20 |
| 31 | sequence_265 | 124 | GACATAGTAGACGATAGTGAA | Forward Primer | 21 |
| 31 | sequence_266 | 125 | CAGACACTAATAGTAATGCAAGTGC | Probe | 25 |
| 31 | sequence_267 | 126 | TTTGCCTGTGAATTACT | Reverse Primer | 17 |
| 32 | sequence_379 | 127 | CGGGTGGTCAGGTAATA | Forward Primer | 17 |
| 32 | sequence_380 | 128 | CTACATCTGTGTTTAGCAGCAAC | Probe | 23 |
| 32 | sequence_381 | 129 | CCAAGTGCTGCCTAATAAT | Reverse Primer | 19 |
| 33 | sequence_409 | 130 | GTTCATGAAGGAATACGAACAT | Forward Primer | 22 |
| 33 | sequence_410 | 131 | CTGCATCATCTTTAAACTGCAC | Probe | 22 |
| 33 | sequence_411 | 132 | GCATGAACTTCCCATACTTTAT | Reverse Primer | 22 |
| 34 | sequence_478 | 133 | TACAGGCGGACGCACTG | Forward Primer | 17 |
| 34 | sequence_479 | 134 | AGATACACTTGCTCCTGTAAGAC | Probe | 23 |
| 34 | sequence_480 | 135 | CCCACAGGATCTACTGTTAAA | Reverse Primer | 21 |
| 35 | sequence_496 | 136 | ACAATGCGACACAAACG | Forward Primer | 17 |
| 35 | sequence_497 | 137 | TATAAAGTTGGGTAGCCGATGC | Probe | 22 |
| 35 | sequence_498 | 138 | CTGCCTGTTTGCATGTT | Reverse Primer | 17 |
| 36 | sequence_526 | 139 | GACACAAACGTTCTGCAA | Forward Primer | 18 |
| 36 | sequence_527 | 140 | TATAAAGTTGGGTAGCCGATGC | Probe | 22 |
| 36 | sequence_528 | 141 | ACATGTACCTGCCTGTT | Reverse Primer | 17 |
| 37 | sequence_538 | 142 | ATTCCATTGGGAACAAGGC | Forward Primer | 19 |
| 37 | sequence_539 | 143 | AGATACACTTGCTCCTGTAAGAC | Probe | 23 |
| 37 | sequence_540 | 144 | GGGCCCACAGGATCTAC | Reverse Primer | 17 |
| 38 | sequence_550 | 145 | CACTGGGTATATTCCATTGGG | Forward Primer | 21 |
| 38 | sequence_551 | 146 | AGATACACTTGCTCCTGTAAGAC | Probe | 23 |
| 38 | sequence_552 | 147 | ATCAGAAGGGCCCACAG | Reverse Primer | 17 |
| 39 | sequence_688 | 148 | AGGATGGTGATATGGTTGATAC | Forward Primer | 22 |
| 39 | sequence_689 | 149 | CTTTGGTGCTATGGACTTTACTAC | Probe | 24 |
| 39 | sequence_690 | 150 | TATCCAGTGGAACTTCACTTT | Reverse Primer | 21 |
| 40 | sequence_724 | 151 | CTTTACTACATTACAGGCTAAC | Forward Primer | 22 |
| 40 | sequence_725 | 152 | CAAATATCCAGTGGAACTTCAC | Probe | 22 |
| 40 | sequence_726 | 153 | TGGTTCTGACACCATTT | Reverse Primer | 17 |
| 41 | sequence_730 | 154 | CAGGGTATTTAGAATACATTTACCTGAC | Forward Primer | 28 |
| 41 | sequence_731 | 155 | TATAATCCAGATACACAGCGGC | Probe | 22 |
| 41 | sequence_732 | 156 | TACACAGGCCCAAACCA | Reverse Primer | 17 |
| 42 | sequence_733 | 157 | TTATAATCCAGATACACAGC | Forward Primer | 20 |
| 42 | sequence_734 | 158 | CTGTGTAGGTGTTGAGGTAGG | Probe | 21 |
| 42 | sequence_735 | 159 | CTAATGCCCACACCTAAT | Reverse Primer | 18 |
| 43 | sequence_739 | 160 | GTTCCTAAAGTATCAGGATT | Forward Primer | 20 |
| 43 | sequence_740 | 161 | CAGGGTATTTAGAATACATTTACCTGACCC | Probe | 30 |
| 43 | sequence_741 | 162 | AAATGAGGTGTCAGGAA | Reverse Primer | 17 |
| 44 | sequence_757 | 163 | TGCAAATTTAGCCAGTTCA | Forward Primer | 19 |
| 44 | sequence_758 | 164 | CTACACCTAGTGGTTCTATGGTTAC | Probe | 25 |
| 44 | sequence_759 | 165 | GCTCGTTGTAACCAATAAGG | Reverse Primer | 20 |
| 45 | sequence_766 | 166 | CATTAAAGGCTCTGGGTCTA | Forward Primer | 20 |
| 45 | sequence_767 | 167 | CTGCAAATTTAGCCAGTTCA | Probe | 20 |
| 45 | sequence_768 | 168 | GAACCACTAGGTGTAGGAAA | Reverse Primer | 20 |
| 46 | sequence_820 | 169 | ATGTTTCAGGACCCACAG | Forward Primer | 18 |
| 46 | sequence_821 | 170 | CCAGAAAGTTACCACAGTTATGC | Probe | 23 |
| 46 | sequence_822 | 171 | CATGTATAGTTGTTTGCAGCTC | Reverse Primer | 22 |
| 47 | sequence_844 | 172 | GGTTACACATTTACAAGCAACT | Forward Primer | 22 |
| 47 | sequence_845 | 173 | ACTAAGGGCGTAACCGAAAT | Probe | 20 |
| 47 | sequence_846 | 174 | CTAACCGGTTTCGGTTCAA | Reverse Primer | 19 |
| 48 | sequence_847 | 175 | CGCTGTTAGGCACATATT | Forward Primer | 18 |
| 48 | sequence_848 | 176 | CCTTAGAAGTTTAAACCTTATGCC | Probe | 24 |
| 48 | sequence_849 | 177 | TGAACTAGGGTGACATTTAGT | Reverse Primer | 21 |
| 49 | sequence_859 | 178 | CGCCAACGCCTTACATA | Forward Primer | 17 |
| 49 | sequence_860 | 179 | CCGCTGTTAGGCACATATTT | Probe | 20 |
| 49 | sequence_861 | 180 | CCTTAGAAGTTTAAACCTTATGCCA | Reverse Primer | 25 |

TABLE 3
Sequences of the Primer and Probe Sets Selected for the CHAMP-16 Assay Pool.
Synthetic primers corresponding to the 9 targeted regions were procured
from Integrated DNA Technologies, with a custom formulation of 500 uM
in buffer IDTE, pH 8.0. FAM - MGBNFQ and VIC - MGBNFQ labeled probes were
procured from ThermoFisher Scientific. The sequences are as shown below:

| | | | | | | Single |
|---|---|---|---|---|---|---|
| | | | | | | Color |
| | | Final | | | Final | Pools |
| | | Conc. | | | Conc. | Nomen- |
| Primer | Sequence (5′ to 3′) | (nM) | Probe | Sequence (5′ to 3′) | (nM) | clature |
| 5_seq_70 | GGAACAACATTAGAAC | 422 | 5_seq_71_ | CAACAAACCGTTGTG | 117 | Pool- |
| _F | AGCAA | | FAM_F | TGATTTG | | 5v39 |
| | (SEQ ID NO: 1) | | | (SEQ ID NO: 3) | | FAM |
| 5_seq_72 | TTCTTCAGGACACAGT | 422 | | | | |
| _R | GG | | | | | |
| | (SEQ ID NO: 2) | | | | | |
| 6_seq_10 | GAGAGGGTGTTAGTGA | 422 | 6_seq_110_ | CACTATATGCCAAAC | 117 | |
| 9_F | AAGA | | FAM_F | ACCACTTAC | | |
| | (SEQ ID NO: 4) | | | (SEQ ID NO: 6) | | |
| 6_seq_11 | GCTAACATTGCTGCCTT | 422 | | | | |
| 1_R | TGC | | | | | |
| | (SEQ ID NO: 5) | | | | | |
| 7_seq_18 | GTAATAAATCAACGTG | 422 | 7_seq_188_ | TGGTGTATTGCTGCA | 117 | |
| 7_F | TTGCG | | FAM_F | TTTGG | | |
| | (SEQ ID NO: 7) | | | (SEQ ID NO: 9) | | |
| 7_seq_18 | GTCAGCTATACTGGGT | 422 | | | | |
| 9_R | GTAA | | | | | |
| | (SEQ ID NO: 8) | | | | | |
| 16_seq_6 | GGCATTAGTGGCCATC | 422 | 16_seq_674 | AAATGCTAGTGCTTA | 117 | |
| 73_F | CTT | | _FAM_F | TGCAGCAA | | |
| | (SEQ ID NO: 10) | | | (SEQ ID NO: 12) | | |
| 16_seq_6 | CTATTATCCACACCTGC | 422 | | | | |
| 75_R | ATTTGC | | | | | |
| | (SEQ ID NO: 11) | | | | | |
| 19_seq_8 | AGTTCTATGTCAGCAA | 422 | 19_seq_815 | CTTGTACGTTTCCTGC | 117 | |
| 14_F | CTATGG | | _FAM_F | TTGC | | |
| | (SEQ ID NO: 13) | | | (SEQ ID NO: 15) | | |
| 19_seq_8 | CAGTGCAGGTCAGGAA | 422 | | | | |
| 16_R | A | | | | | |
| | (SEQ ID NO: 14) | | | | | |
| 2_seq_34 | CTAACATTAATGCTGGT | 422 | 2_seq_344_ | TCTAGGTGGCCTTATT | 117 | Pool- |
| 3_F | ACAGA | | VIC_F | TACA | | 4v20 |
| | (SEQ ID NO: 16) | | | (SEQ ID NO: 18) | | VIC |
| 2_seq_34 | GAAATGTAAACACCAC | 422 | | | | |
| 5_R | CAATC | | | | | |
| | (SEQ ID NO: 17) | | | | | |
| 4_seq_55 | AGAACACGTAGAGAAA | 422 | 4_seq_56_ | TCATGCAATGTAGGT | 117 | |
| _F | CCC | | VIC_R | GTATCTCC | | |
| | (SEQ ID NO: 19) | | | (SEQ ID NO: 21) | | |
| 4_seq_57 | GAGATCAGTTGTCTCTG | 422 | | | | |
| _R | GTT | | | | | |
| | (SEQ ID NO: 20) | | | | | |
| 13_seq_5 | CTACACATAATAATCC | 422 | 13_seq_509 | CATCTGTATTGCAGC | 117 | |
| 08_F | CACTTTCAC | | _VIC_F | CTCCA | | |
| | (SEQ ID NO: 22) | | | (SEQ ID NO: 24) | | |
| 13_seq_5 | AAATGCCCTCCAGTTTC | 422 | | | | |
| 10_R | T | | | | | |
| | (SEQ ID NO: 23) | | | | | |
| 15_seq_5 | CCAGCATTAACCTCTA | 422 | 15_seq_593 | ATTCTACTGTACCTA | 117 | |
| 92_F | GGC | | _VIC_R | ATGCCAGTAC | | |
| | (SEQ ID NO: 25) | | | (SEQ ID NO: 27) | | |
| 15_seq_5 | CGAGTACGTAGTGTTT | 422 | | | | |
| 94_R | GTTTATTAC | | | | | |
| | (SEQ ID NO: 26) | | | | | |

TABLE 4
Sequences of the Primer and Probe Sets Selected for the CHAMP-hr Assay
Pool.

| | | | | | | Single |
|---|---|---|---|---|---|---|
| | | | | | | Color |
| | | Final | | | Final | Pools |
| | | Conc. | | | Conc. | Nomen |
| Primer | Sequence (5′ to 3′) | (nM) | Probe | Sequence (5′ to 3′) | (nM) | clature |
| HPV31_4 | GACCTCCACTGTTAT | 422 | HPV31_FAM | TACCCGACAGCTCAG | 117 | hrHPV- |
| F | GAGCAA | | _6PrF | (SEQ ID NO: 38) | | 31_35_ |
| | (SEQ ID NO: 37) | | | | | 39_ |
| HPV31_5 | CTATGACATCCTCCTC | 422 | | | | Pool4 |
| R | ATCTGA | | | | | FAM |
| | (SEQ ID NO: 39) | | | | | |
| HPV35_1 | AGGTCGGTGTATGTC | 422 | HPV35_FAM | ACGTGTTGGTTTCCAA | 117 | |
| F | CTGTTG | | _3PrR | C | | |
| | (SEQ ID NO: 40) | | | (SEQ ID NO: 41) | | |
| HPV35_2 | TGATTACACCTCGGT | 422 | | | | |
| R | TTCTC | | | | | |
| | (SEQ ID NO: 42) | | | | | |
| HPV39_7 | CGGGATACTCTGCGA | 422 | HPV39_FAM | CAGCAGCTGTTTATGG | 117 | |
| F | CAACT | | _9PrF | ACTCA | | |
| | (SEQ ID NO: 43) | | | (SEQ ID NO: 44) | | |
| HPV39_8 | CACACCACGGACACA | 422 | | | | |
| R | CAAAT | | | | | |
| | (SEQ ID NO: 45) | | | | | |
| HPV18_4 | ACCACAATACTATGG | 422 | HPV18_VIC | TGAGGATCCAACACG | 117 | hrHPV- |
| F | CGCGC | | _6PrF | G | | 18_33_ |
| | (SEQ ID NO: 46) | | | (SEQ ID NO: 47) | | Pool1 |
| HPV18_5 | GGTAGCTTGTAGGGT | 422 | | | | VIC |
| R | CGCCG | | | | | |
| | (SEQ ID NO: 48) | | | | | |
| HPV33_4 | AAACTGCACTGTGAC | 422 | HPV33_VIC | AAACGCCATGAGAGG | 117 | |
| F | GTGTA | | _6PrF | AC | | |
| | (SEQ ID NO: 49) | | | (SEQ ID NO: 50) | | |
| HPV33_5 | CTTTAACGTTGGCTTG | 422 | | | | |
| R | TGTC | | | | | |
| | (SEQ ID NO: 51) | | | | | |

TABLE 5
Sequences of the Primer and Probe Sets
Selected for the Reference Gene
RPP30.

| | | Final | | | Final |
|---|---|---|---|---|---|
| | | Conc. | | Sequence | Conc. |
| Primer | Sequence (5′ to 3′) | (nM) | Probe | (5′ to 3′) | (nM) |
| RPP30_ | AGATTTGGACCTGCGA | 844 | RPP30_ | CCGCGCAGA | 234 |
| F | GC (SEQ ID NO: 58) | | FAM | GCCTTC | |
| RPP30_ | GGAGCGGCTGTCTCCA | 844 | | (SEQ ID | |
| R | CAAG (SEQ ID NO: 59) | | | NO: 60) | |

TABLE 6
Sequences of the Primer and Probe Sets
Selected for the Plant Spike.

| | | Final | | | Final |
|---|---|---|---|---|---|
| | Sequence | Conc. | | Sequence | Conc. |
| Primer | (5′ to 3′) | (nM) | Probe | (5′ to 3′) | (nM) |
| Plant_ | GATCTTCAACCAGGAG | 844 | Plant_ | ACCCATCT | 234 |
| Spike_F | ATCA (SEQ ID NO: 181) | | Spike_ | TCACCGGA | |
| Plant_ | AGTGACAGTGAGGACA | 844 | VIC | (SEQ ID | |
| Spike_R | ATCC (SEQ ID NO: 182) | | | NO: 183) | |

TABLE 7
Synthetic Target DNA Sequences Corresponding to the CHAMP-16 Assay.
Synthetic dsDNA templates corresponding to each of the
9 targeted regions of HPV-16 for the CHAMP-16 assay
were procured from Integrated DNA Technologies (location).
The + strand sequences are as shown below with the
underlined sequences specific to HPV16 and non-
underlined flanking sequences (AATGC and TCACT)
to distinguish the synthetic fragments
from HPV16 genomic DNA.

| | | | | Length |
|---|---|---|---|---|
| | | | | (bp) |
| | | | | Specific to |
| Name | Sequence (underlined = specific to HPV16) | Start 5′ | End 3′ | HPV16 |
| Seq_5 | AATGCGGAACAACATTAGAACAGCAATACAACA | 7398 | 7487 | 90 |
| | AACCGTTGTGTGATTTGTTAATTAGGTGTATTAAC | | | |
| | TGTCAAAAGCCACTGTGTCCTGAAGAATCACT | | | |
| | (SEQ ID NO: 28) | | | |
| Seq_6 | AATGCGAGAGGGTGTTAGTGAAAGACACACTATA | 530 | 626 | 97 |
| | TGCCAAACACCACTTACAAATATTTTAAATGTACT | | | |
| | AAAAACTAGTAATGCAAAGGCAGCAATGTTAGCT | | | |
| | CACT | | | |
| | (SEQ ID NO: 29) | | | |
| Seq_7 | AATGCGTAATAAATCAACGTGTTGCGATTGGTGT | 683 | 747 | 65 |
| | ATTGCTGCATTTGGACTTACACCCAGTATAGCTGA | | | |
| | CTCACT | | | |
| | (SEQ ID NO: 30) | | | |
| Seq_ | AATGCGGCATTAGTGGCCATCCTTTATTAAATAA | 5120 | 5205 | 86 |
| 16 | ATTGGATGACACAGAAAATGCTAGTGCTTATGCA | | | |
| | GCAAATGCAGGTGTGGATAATAGTCACT | | | |
| | (SEQ ID NO: 31) | | | |
| Seq_ | AATGCAGTTCTATGTCAGCAACTATGGTTTAAACT | 6637 | 6721 | 85 |
| 19 | TGTACGTTTCCTGCTTGCCATGCGTGCCAAATCCC | | | |
| | TGTTTTCCTGACCTGCACTGTCACT | | | |
| | (SEQ ID NO: 32) | | | |
| Seq_2 | AATGCCTAACATTAATGCTGGTACAGATTCTAGG | 1697 | 1765 | 69 |
| | TGGCCTTATTTACATAATAGATTGGTGGTGTTTAC | | | |
| | ATTTCTCACT | | | |
| | (SEQ ID NO: 33) | | | |
| Seq_4 | AATGCAGAACACGTAGAGAAACCCAGCTGTAATC | 7575 | 7669 | 95 |
| | ATGCATGGAGATACACCTACATTGCATGAATATA | | | |
| | TGTTAGATTTGCAACCAGAGACAACTGATCTCTC | | | |
| | ACT | | | |
| | (SEQ ID NO: 34) | | | |
| Seq_ | AATGCCTACACATAATAATCCCACTTTCACTGACC | 3839 | 3914 | 76 |
| 13 | CATCTGTATTGCAGCCTCCAACACCTGCAGAAAC | | | |
| | TGGAGGGCATTTTCACT | | | |
| | (SEQ ID NO: 35) | | | |
| Seq_ | AATGCCCAGCATTAACCTCTAGGCGTACTGGCAT | 4246 | 4316 | 71 |
| 15 | TAGGTACAGTAGAATTGGTAATAAACAAACACTA | | | |
| | CGTACTCGTCACT | | | |
| | (SEQ ID NO: 36) | | | |

TABLE 8
Synthetic Target DNA Sequences Corresponding to the CHAMP-hr Assay.
Synthetic dsDNA templates used for testing the performance of the high-risk HPV subtype
specific primer/probe sets were procured from Integrated DNA Technologies (location). The +
strand sequences are as shown below with the underlined sequences specific to HPV16 and
non-underlined flanking sequences (AATGC and TCACT) to distinguish the synthetic
fragments from HPV16 genomic DNA.

| | | Length |
|---|---|---|
| | | (bp) |
| | | Specific to |
| | | hrHPV |
| Name | Sequence (underlined = specific to respective HPV) | Subtype |
| HPV 18_ | AATGCTGTGAGAAACACACCACAATACTATGGCGCGCTTTGAGG | 107 |
| Spike | ATCCAACACGGCGACCCTACAAGCTACCTGATCTGTGCACGGAA | |
| | CTGAACACTTCACTGCAAGACATATCACT | |
| | (SEQ ID NO: 52) | |
| HPV_31_ | AATGCGAGGCAACTGACCTCCACTGTTATGAGCAATTACCCGAC | 116 |
| Spike | AGCTCAGATGAGGAGGATGTCATAGACAGTCCAGCTGGACAAGC | |
| | AGAACCGGACACATCCAATTACAATATCGTTACTCACT | |
| | (SEQ ID NO: 53) | |
| HPV_33_ | AATGCGATTTCATAATATTTCGGGTCGTTGGGCAGGGCGCTGTGC | 122 |
| Spike | GGCGTGTTGGAGGTCCCGACGTAGAGAAACTGCACTGTGACGTG | |
| | TAAAAACGCCATGAGAGGACACAAGCCAACGTTAAAGGTCACT | |
| | (SEQ ID NO: 54) | |
| HPV_35_ | AATGCAAACGATTCCATAACATCGGTGGACGGTGGACAGGTCGG | 141 |
| Spike | TGTATGTCCTGTTGGAAACCAACACGTAGAGAAACCGAGGTGTA | |
| | ATCATGCATGGAGAAATAACTACATTGCAAGACTATGTTTTAGAT | |
| | TTGGAACCCGAGGTCACT | |
| | (SEQ ID NO: 55) | |
| HPV_39_ | AATGCGAACCACAGCGTCACACAATACAGTGTTCGTGTTGTAAG | 142 |
| Spike | TGTAACAACACACTGCAGCTGGTAGTAGAAGCCTCACGGGATAC | |
| | TCTGCGACAACTACAGCAGCTGTTTATGGACTCACTAGGATTTGT | |
| | GTGTCCGTGGTGTGTCACT | |
| | (SEQ ID NO: 56) | |

TABLE 9
Synthetic Target DNA Sequence Used for
Testing to the Plant Spike Assay.

| | | Length |
|---|---|---|
| Name | Sequence | (bp) |
| Plant_ | TGTTTTCTTTAGTACTCAGAAAGGTTGTGTAG | 150 |
| Spike_ | GATTGTTGAGAGTGTTGGAGAAGGAGTGACTG | |
| 150bp | ATCTTCAACCAGGAGATCATGTTTTACCCATC TTCACCGGAGAGTGTGGGGATTGTCCTCACTG | |
| | TCACTCCGAGGAATCCAACATG | |
| | (SEQ ID NO: 57) | |

### 6. EXAMPLES

It will be readily apparent to those skilled in the art that other suitable modifications and adaptations of the methods of the present disclosure described herein are readily applicable and appreciable, and may be made using suitable equivalents without departing from the scope of the present disclosure or the aspects and embodiments disclosed herein. Having now described the present disclosure in detail, the same will be more clearly understood by reference to the following examples, which are merely intended only to illustrate some aspects and embodiments of the disclosure, and should not be viewed as limiting to the scope of the disclosure. The disclosures of all journal references, U.S. patents, and publications referred to herein are hereby incorporated by reference in their entireties.

The present disclosure has multiple aspects, illustrated by the following non-limiting examples.

### Example 1

Subject Recruitment, Specimen Collection and Processing. Subjects ≥18 years old with HPV+ OPSCC were recruited and provided with informed consent. p16 was determined by tumor immunohistochemistry and was used as a surrogate marker for HPV status of a subject's cancer. Enrolled subjects had baseline collection of clinical data on demographics, disease characteristics, treatment plan, and previously obtained biomarker testing. Blood was collected prior to the initiation of definitive therapy (radiation+/−chemotherapy or surgery) every 3 months after completion of therapy. Plasma was collected in Streck or PaxGene tubes designed for ctDNA preservation and was isolated using two sequential centrifugation steps per manufacturer recommendations as described previously [29]. Collection continued for 3 years, until subject withdrawal of consent, or at investigator discretion. Surveillance imaging and office visits were obtained at the frequency as determined by the discretion of the treating physician. The timing of the imaging and corresponding results were recorded in the database. Extraction of gDNA from cell lines and gDNA fragmentation is described below.

Extraction and Fragmentation of Genomic DNA. Genomic DNA from HPV16 positive cell line UM-SCC-104 and HPV18 positive cell line UM-SCC-105 was extracted using the Wizard DNA Purification Kit protocol (Promega #A1120). Restriction digest of UM-SCC-104 gDNA and non-HPV human genomic DNA (hgen DNA) (Promega #G3041) was carried out using HindIII (New England Biolabs #R0104S) by incubation at 37° C. overnight at supplier recommended concentrations, followed by incubation at 80° C. for 20 minutes to inactivate the enzyme. Shearing of the UM-SCC-104 gDNA, UM-SCC-105 gDNA and hgen DNA (Promega #G3041) was carried out at the Advanced Genomics Core (University of Michigan) using a Covaris S2 Focus Ultrasonicator. Sample volume was kept constant at 130 μl with a concentration ranging from 10 ng/μl to 50 ng/μl of DNA. Sheared DNA was in a size range of 50 bp-500 bp, with the majority between 150 bp-300 bp. 16 of 17 selected primer/probe sets (except primer/probe set #8) from the primary screen with non-sheared UM-SCC-104 gDNA (FIG. 1B) passed the screen with sheared gDNA and no detectable cross-reactivity with sheared non-HPV hgen DNA was observed (data not shown). Based on these results, sheared UM-SCC-104 gDNA was used as a template for all further screening experiments to develop the multi-probe assay.

CHAMP-16 Assay Development and Droplet Digital PCR. The assay was developed through a combination of bioinformatics-based probe selection (Table 10) [35-39], empirical validation, and optimization of the PCR conditions (Table 1-Table 9, Table 11-Table 19) [40-42]. Specific parameters for primer/probe screens using ddPCR, serial dilutions and the calculations used for LoD are described below.

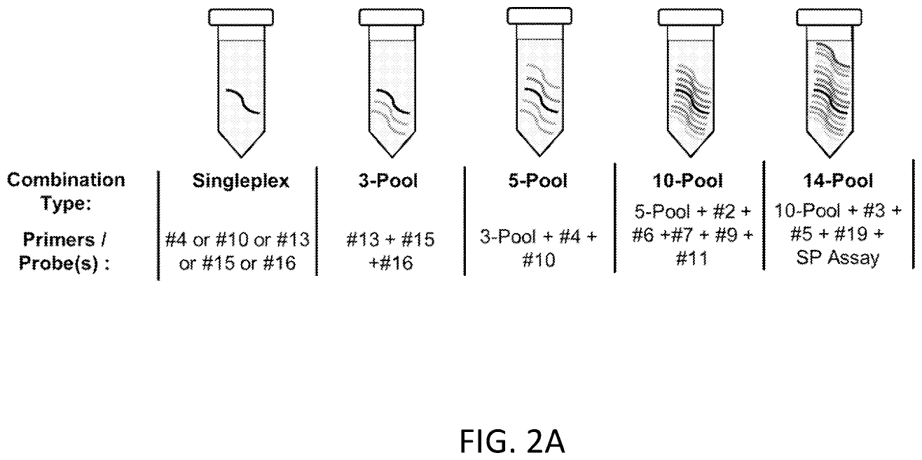

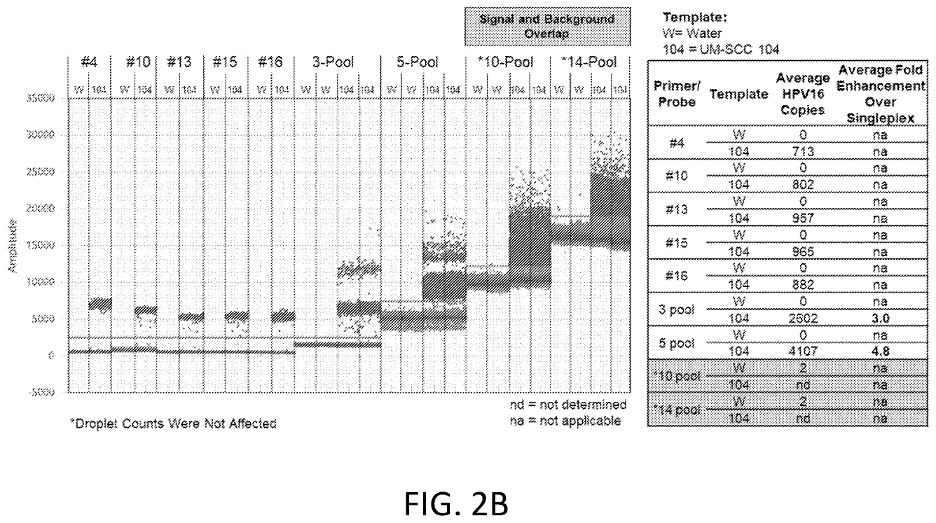

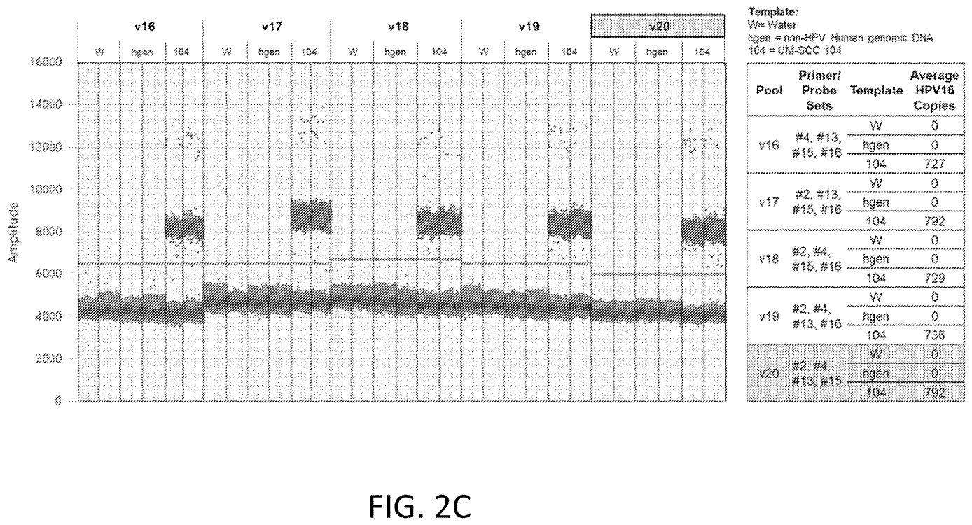

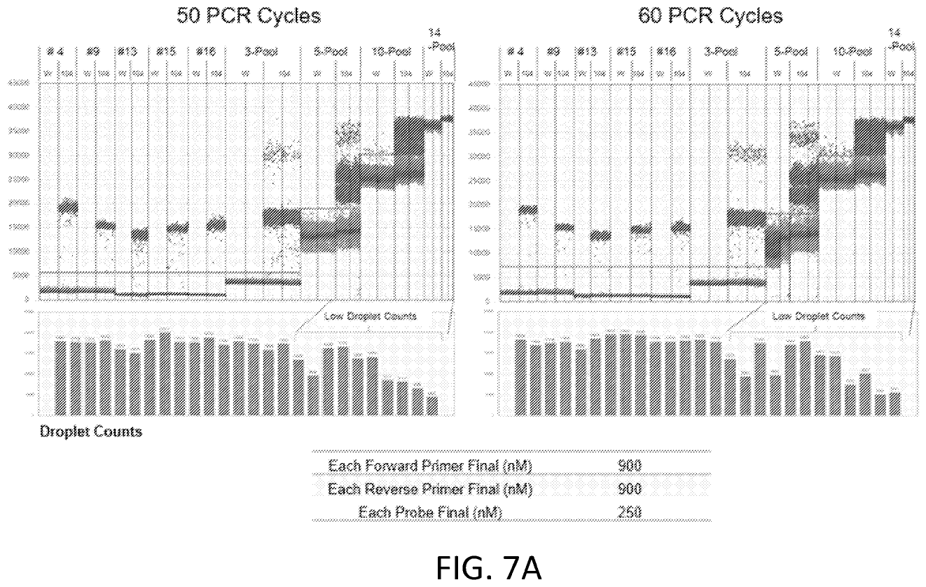

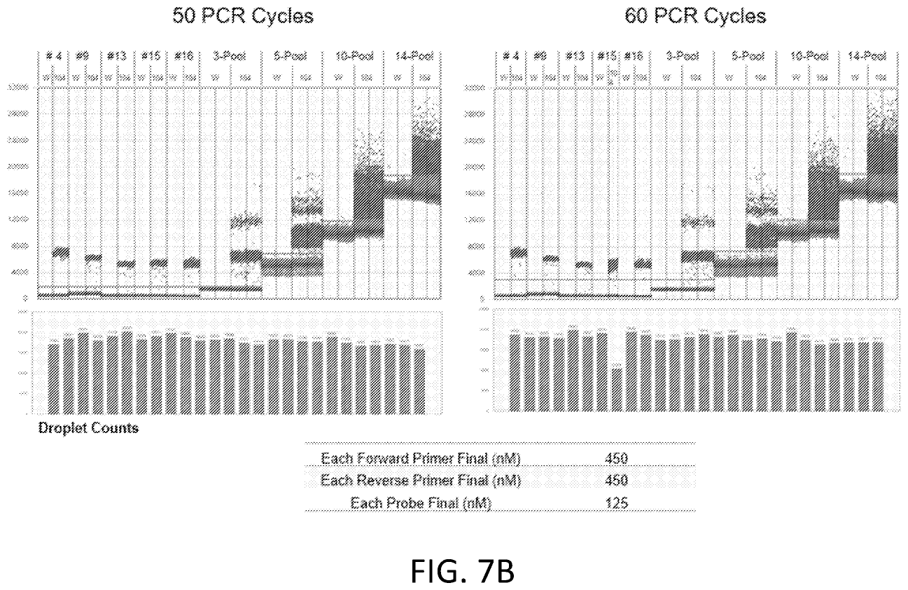

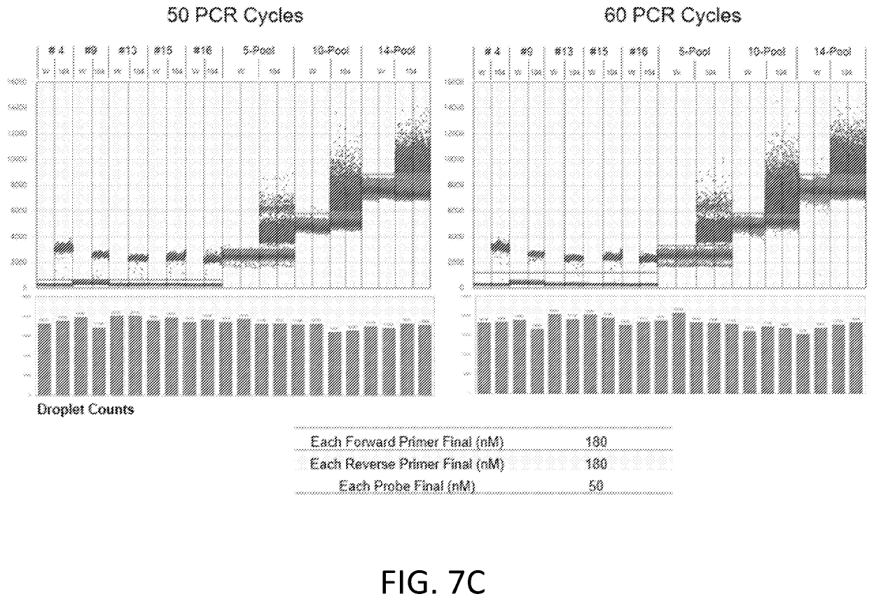

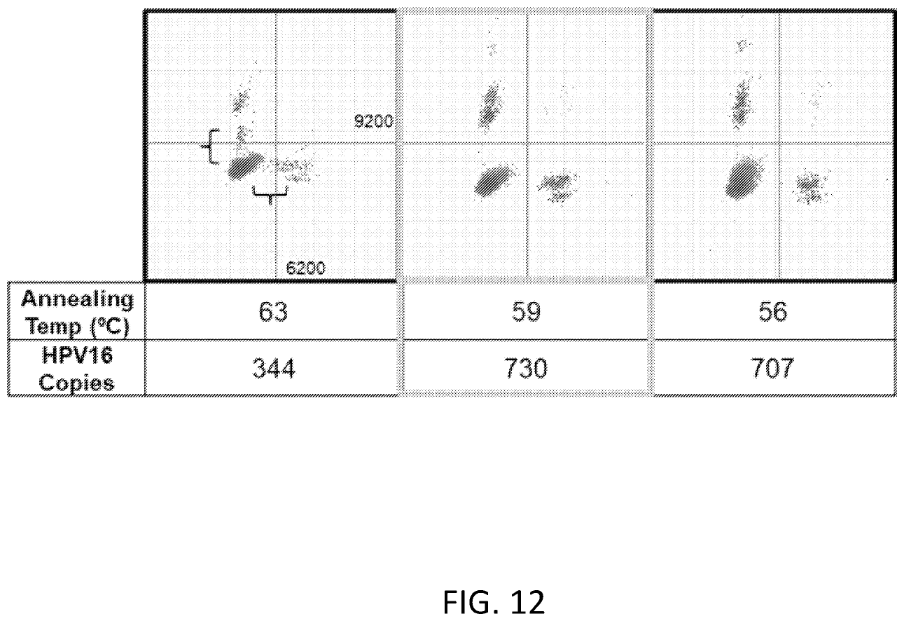

Droplet Digital PCR. Each ddPCR reaction contained template DNA, 2×ddPCR Supermix for Probes (no dUTP) (Bio-Rad #186-3024), HPV16 primer/probe assay mix, and nuclease free water (see supplementary tables for final concentrations and volumes). The reaction mix was partitioned using QX200 droplet generator (Bio-Rad), transferred into a 96-well plate, sealed, and cycled in a C1000 Thermal Cycler (Bio-Rad). Droplets were read using QuantaSoft Software in the QX200 reader (Bio-Rad). For the individual primer/probe assay testing set up during screening, annealing temperature was set to 58° C. based on the lowest primer/probe melting temperature from the selected list of 19 combinations and PCR was performed for 40 cycles and ramp rate of 2° C./second as per standard ddPCR protocols. For the development of the multi-probe pool, the ramp rate was set to 1° C./second for each step, annealing temperature set to 60° C. and cycle number set to 50. Once the pool was finalized, the PCR conditions were optimized for the pool for analytical and clinical validation with annealing temperature set to 59° C. (FIG. 12). Sheared UM-SCC-104 (HPV16+) cell line gDNA was included as the positive control. UM-SCC-105 (HPV18+) cell line gDNA and 44,000 diploid GEs of sheared non-HPV hgen DNA (Promega #G3041) were used as negative controls. Thresholds for distinguishing positive and negative droplets were standardized for each assay. In cases where 10 or more primer/probe sets were pooled, the background could not be distinguished from the signal (FIG. 2B, FIG. 7A, FIG. 7B and FIG. 7C) and positive droplets could not be determined.

Calculation of Limit of Detection and Coefficient of Variation for the CHAMP-16 Assay. Limit of Blank (LoB) for the CHAMP-16 assay was calculated using the formula: LoB=mean_{blank}+1.645(Standard Deviation_{blank}) and determined to be 0.48 copies per 20 μl reaction using ~44,000 diploid GEs of sheared non-HPV hgen DNA as blank (n=61). Limit of Detection (LoD) for the CHAMP-16 assay was calculated using the formula: LoD=LoB+1.645(Standard Deviation_{low concentration sample}).

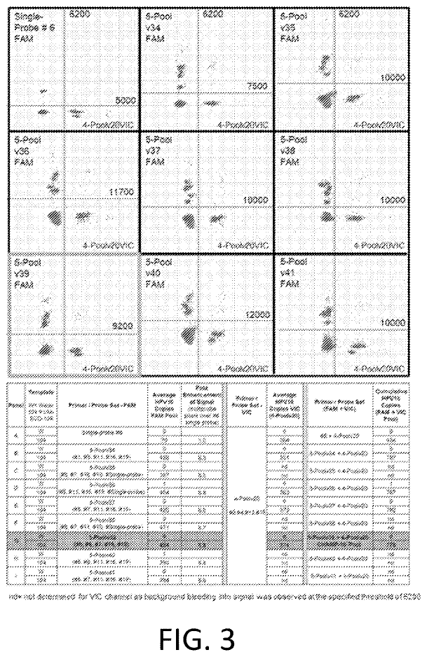

To determine the LoD using UM-SCC-104 cell line gDNA, 12 serially diluted samples of the gDNA starting at 140 haploid GEs (FIG. 3K) were analyzed. The standard deviation was determined to be 1.3 copies for measurement of the lowest concentration sample. Based on this value and the LoB determined above, the LoD using UM-SCC-104 cell line gDNA was determined to be 2.6 copies per 20 μl reaction.

To determine the LoD using the synthetic HPV16 DNA pool of 9 targets, 12 serially diluted samples of this DNA pool starting at 4000 copies total (FIG. 3J) were analyzed. The standard deviation was determined to be 2.2 copies for measurement of the lowest concentration sample. Based on this value and the LoB determined above, the LoD using the synthetic HPV16 DNA pool of 9 targets was determined to be 4.1 copies per 20 μl reaction. This value was used as the LoD for patient sample analysis. Coefficient of Variation (CV) was calculated using the formula: % CV=(Standard Deviation/Mean)*100.

### Example 2

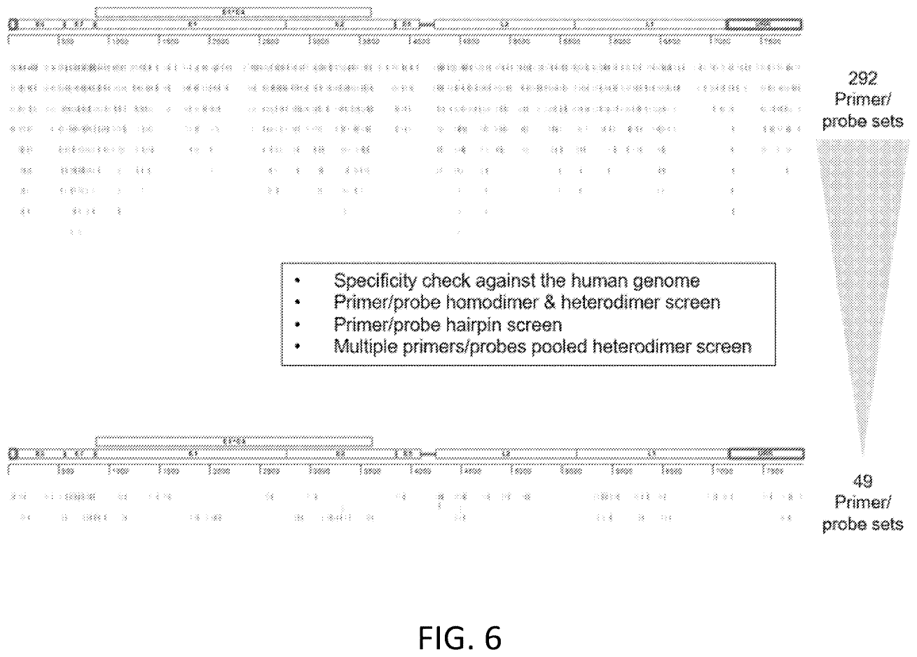

Assay Design: Computational Analysis and Screening of the HPV16 Genome for Candidate Primer/Probe Sets. To design a multi-target assay spanning the entire HPV16 genome, a commonly referenced variant (NC_001526.4) was chosen and the genomic sequence was screened computationally for candidate primers and probes (FIG. 1A). Briefly, the HPV16 genomic sequence was scanned using overlapping 0.8 to 1 kb windows to identify primers and probes with variable length and a range of melting temperatures adaptable to ddPCR conditions ideal for plasma cfDNA assays [35,36]. This identified 292 primer/probe combinations (FIG. 6) that were checked for homology to the human genome using a BLAST algorithm followed by alignment, to eliminate those with high risk of homodimerization, heterodimerization, and hairpin formation [38,39]. This resulted in 49 primer/probe combinations (FIG. 1A and FIG. 6), of which 19 candidates with optimal design for a multi-probe assay were selected for further screening and development (FIG. 1A).

### Example 3

Assay Development: Experimental Screening of Candidate Primer/Probe Combinations using ddPCR. A SP ddPCR assay for detection of HPV16 ctDNA was previously reported targeting a 77 bp region in the E6 gene and the Upper Regulatory Region [29-31]. The SP ddPCR assay has a LoD of 4.2 GEs of HPV16 and was analytically validated in plasma samples of patients with HPV+ OPSCC and HPV− OPSCC for clinical sensitivity and specificity [29]. Since the SP ddPCR primer/probe combination met the criteria used for the computational screens, it was included in the development screens for the multi-probe assay. To test the performance of the 20 primer/probe candidates (19 new primer/probe sets and the published SP assay) in ddPCR, HindIII digested gDNA from the low copy number HPV16 positive cell line UM-SCC-104 was used as a template and the candidate sets were tested as individual assays (FIG. 1B). Seventeen of the 20 primer/probe candidates with 3-fold or higher signal intensity over the background were selected for further screening, while 2 sets with low signal intensity (#17 and #18) and 1 with suboptimal amplification and extensive raindrop pattern (#14) were rejected. None of the combinations tested showed cross-reactivity with saturating amounts (200,000 GEs) of HindIII digested non-HPV hgen DNA control (FIG. 1B), indicating that the selected candidates displayed specificity for detection of HPV16 DNA.

Determination of Upper Limit for Pooling of Multiple Probes. To mimic plasma cfDNA derived from the nuclear genome that has a fragmented size around 166-167 bp [35,46,47], the 17 primer/probe candidates were screened using ultrasonically sheared UM-SCC-104 gDNA and non-HPV hgen DNA (median size 300 bp; see supplementary methods). Shearing of gDNA was also done to avoid potential generation of amplicons longer than the intended target length (<150 bp), which could occur by amplification with primers from different pairs during development of the multi-probe assay. Further, to determine the feasibility of pooling the selected primer/probes and determine the maximum number that can be included in a single reaction [40], pools with varying numbers of FAM labeled primer/probe sets were tested (FIGS. 2A and 2B). Pooling led to an increase in the background in a manner proportional to the number of probes pooled, while the signal from the positive droplets, which theoretically represented only a single amplified target per droplet, did not increase proportionately (FIG. 2B). Hence, a minimum of 2-fold higher signal intensity compared to background was set as a selection criterion (termed ‘the 2-fold criterion’) to evaluate the number of probes that could be pooled to reliably measure the HPV16 copies.

To determine the impact of changing primer/probe concentrations and PCR cycling conditions on the signal intensity and background [41], each pool was tested at the supplier recommended primer/probe concentrations (900 nM/250 nM final) (FIG. 7A) or 2-fold and 5-fold lower primer/probe concentrations (FIGS. 2B, 7B, and 7C). Combinations containing 5 primer sets and probes (termed ‘5-Pool’) passed the 2-fold criterion under different parameters tested (FIGS. 2B, 7A, 7B, and 7C). In the case of 10-Pool and 14-Pool versions, the background was highly elevated as determined by the no-template water blank, resulting in an overlap with the signal (FIGS. 2B, 7A, 7B, and 7C). These results indicated that a maximum of 5 primer/probe sets could be pooled for an acceptable signal intensity over the background to determine HPV16 copies.

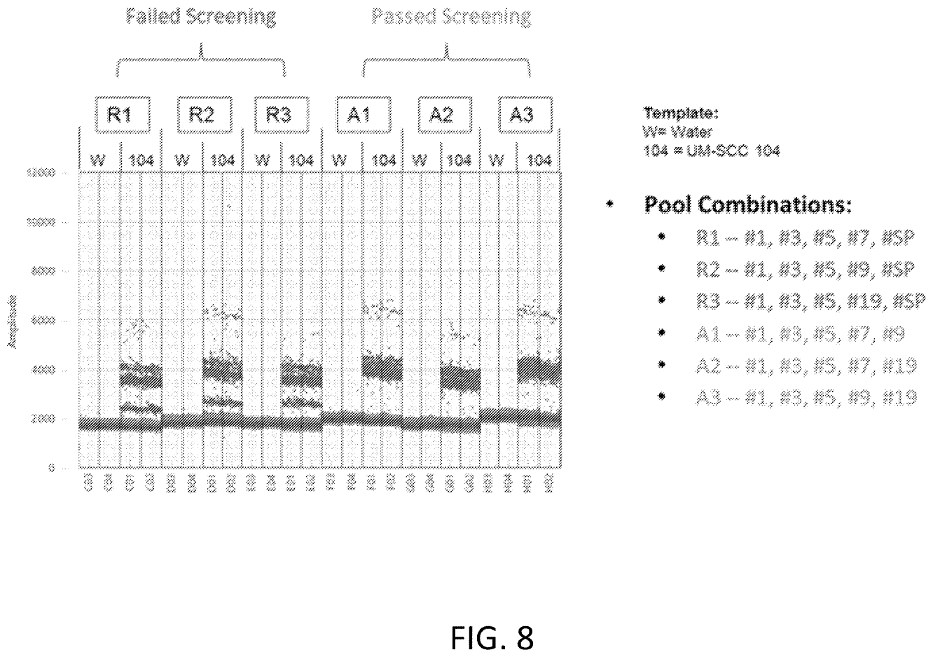

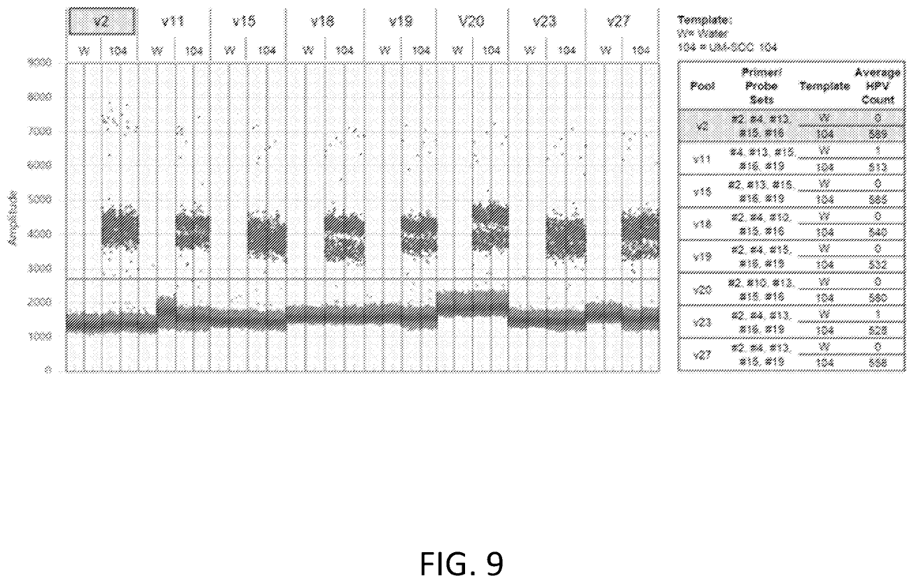

More than 50 different 5-Pool combinations using FAM labeled probes were then screened. Many combinations failed to meet the 2-fold criterion and were rejected (FIG. 8). 6-Pool versions of selected primer/probe combinations which passed the 5-Pool testing were also tried, but they did not meet the 2-fold criterion (data not shown). Additionally, pools containing one or more of these five primer/probe sets, #3, #9, #10, #11 and #12, were not selected for further analysis as the targeted region was located partially or completely in the viral E2 gene (FIG. 1A) which is frequently lost during integration of viral DNA into the host genome [29]. Of the combinations that passed the 2-fold criterion, the pool with the highest number of HPV16 copies measured (5-Poolv2) was chosen for further analysis and development (FIG. 9).

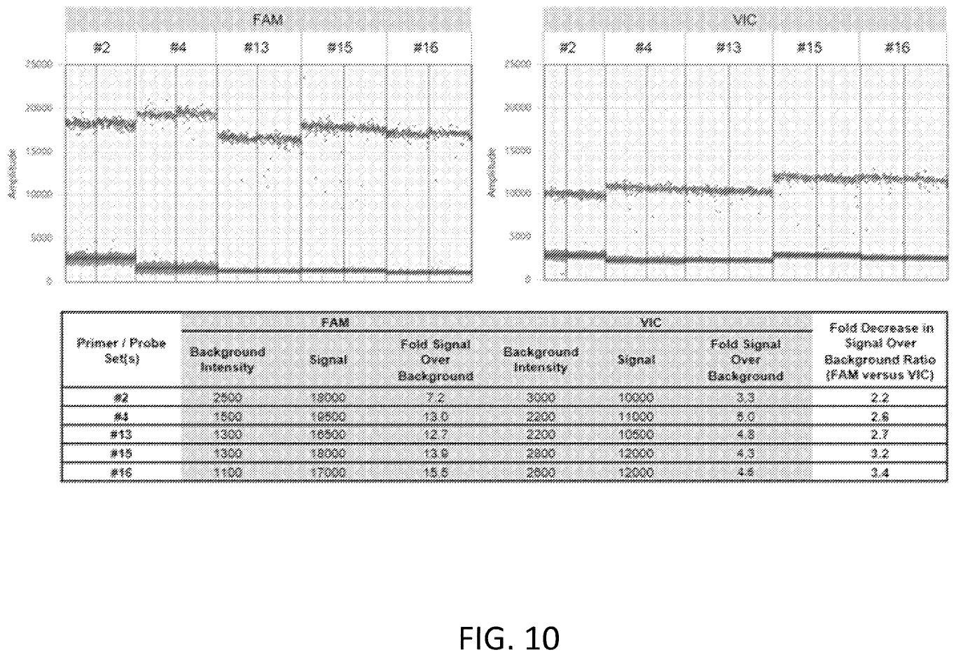

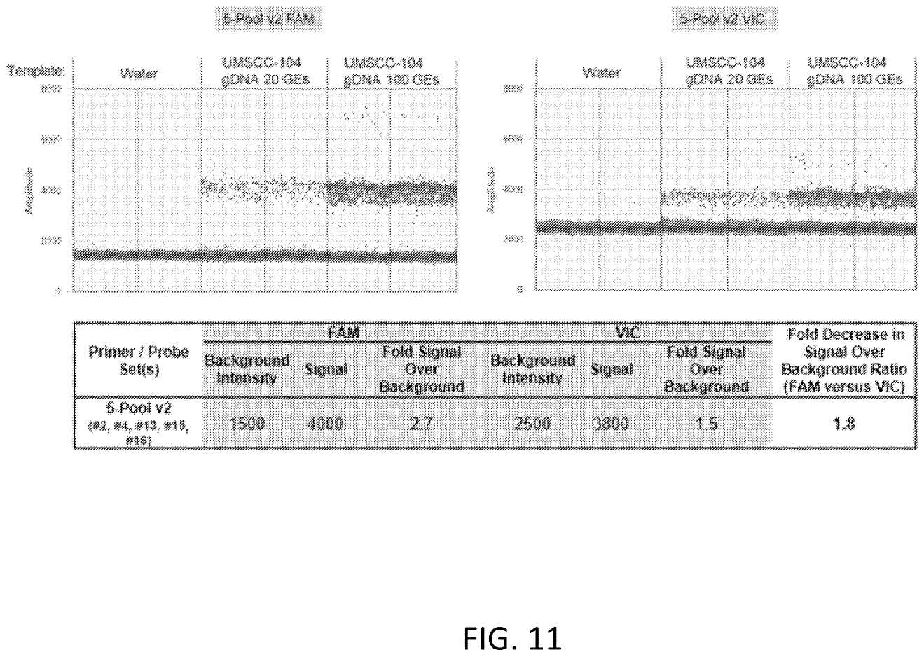

Selection of the Dual-Colored Multi-Probe Pool. To broaden the coverage of HPV16 genome detection, a 5-Pool using VIC labeled probes compatible with the FAM labeled probes was developed and the dual-color detection capabilities of ddPCR, potentially targeting up to 10 distinct regions of the HPV16 genome, was used. VIC labeled probes tend to have a weaker signal intensity compared to FAM [41,42]. Therefore, VIC labeled versions of the FAM pool (5-Poolv2), selected based on the strong signal intensity when compared to background in the candidate screens (FIG. 9) were tested. When tested as individual assays using sheared UM-SCC-104 gDNA, all 5 probes showed a lower signal with the VIC labeled version compared to the FAM labeled version, as assessed by the signal intensity relative to the background (FIG. 10). Consequently, a significant reduction in the signal intensity was observed for the VIC labeled version of 5-Poolv2, falling below the 2-fold criterion (FIG. 11). Hence, 4-Pool combinations were tested instead with the VIC labeled versions of the 5 selected probes comprising 5-Poolv2, and all 4-Pools tested with the VIC labeled probes passed the 2-fold criterion (FIG. 2C). Of the two 4-Pools with the highest HPV16 copies measured, 4-Poolv20VIC was chosen for further development of the dual-colored assay since it included primer/probe #4, which targets the viral E6/E7 junction highly represented in HPV16 ctDNA [29,48,49].

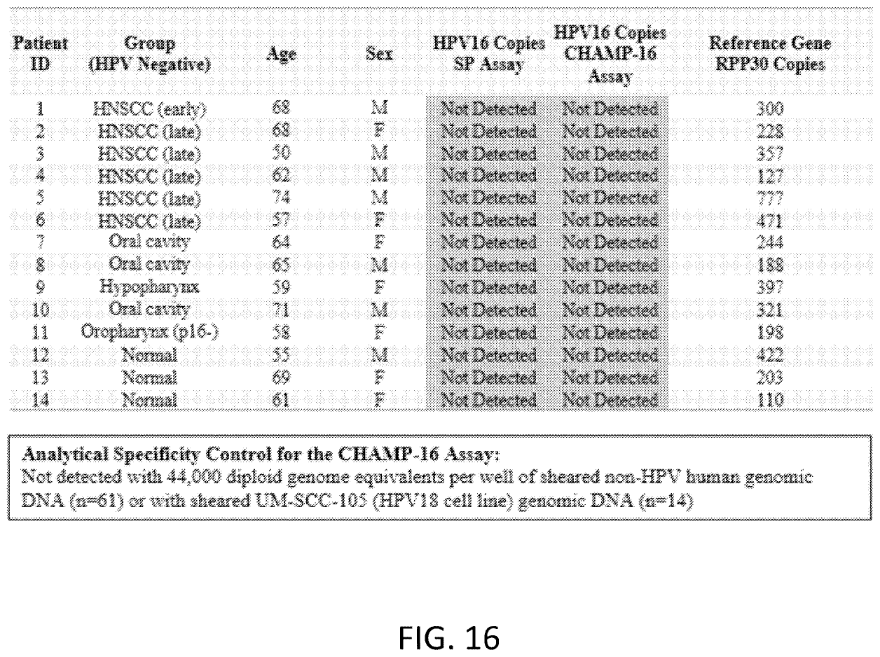

To find a compatible 5-pool containing FAM labeled probes, eight combinations excluding the 4 primer/probe sets used in the 4-Poolv20VIC, were tested for detection of HPV-16 in a dual-color format (FIG. 3). To check for any impact on the background of the VIC pool due to the FAM pool, a single FAM labeled primer/probe set (#6) was also tested in combination with 4-Poolv20VIC (FIG. 3A). Of the eight FAM 5-Pools screened, the background for the VIC labeled pool was altered by four pools, which were rejected (FIGS. 3C, 3F, 3H and 3I). Based on the 2-D droplet plots, 5-Poolv39FAM was selected as the pool compatible with 4-Poolv20VIC (FIG. 3G), since it passed the 2-fold criterion and did not alter the background in the VIC channel when compared to the single-probe set (FIG. 3A). The selected 9-pool (5-Poolv39FAM+4-Poolv20VIC) assay was termed the ‘CtDNA HPV16 Assessment using Multiple Probes’ (CHAMP-16) assay. When tested with sheared non-HPV hgen DNA (~44,000 GEs) or sheared genomic DNA from an HPV18+ cell line UM-SCC-105, the CHAMP-16 assay did not show any signal, indicating that it is specific to HPV16 (FIG. 16).

### Example 4

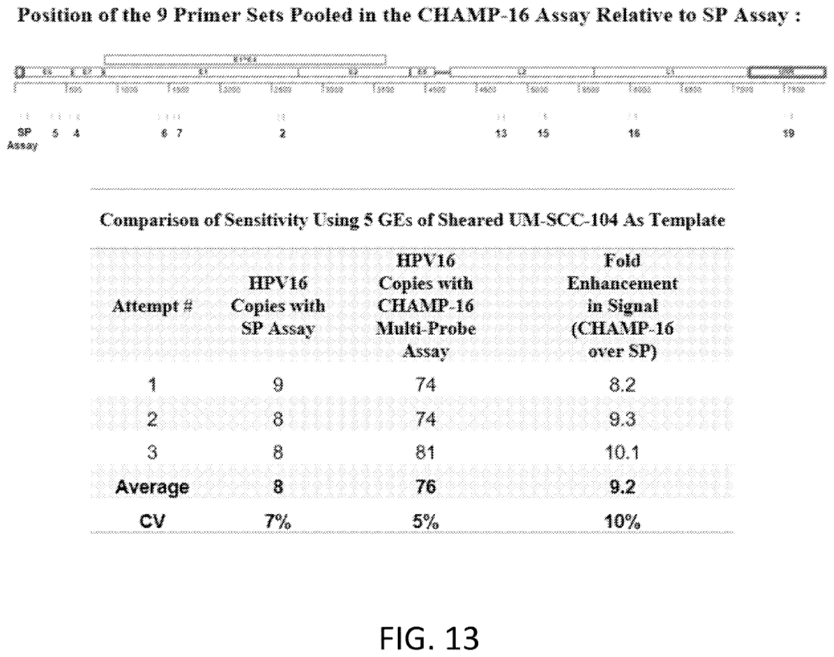

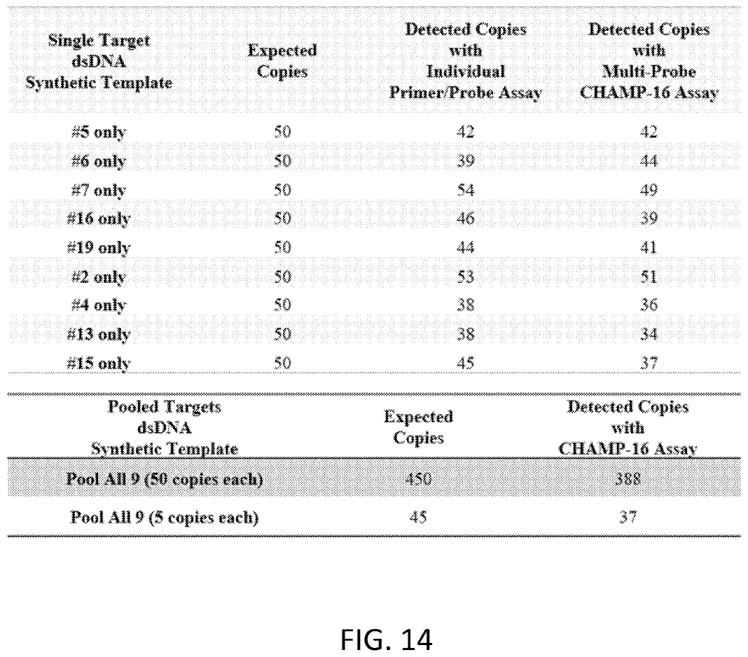

Assay Validation: Analytical Validation of the CHAMP-16 Assay and the CHAMP-hr Assay. Optimal PCR conditions for the CHAMP-16 assay were determined before proceeding with analytical validation (FIG. 12). When tested with sheared UM-SCC-104 gDNA, the CHAMP-16 assay demonstrated an average of 9.5-fold higher analytical sensitivity compared to the SP assay (FIG. 13). To calculate the LoD, the CHAMP-16 assay was tested across a range of concentrations of a pool of chemically synthesized HPV16 DNA fragments corresponding to the 9 targeted sites (Table 6, FIG. 13), as well as across serial dilutions of sheared UM-SCC-104 gDNA as templates. Each of the 9 synthetic DNA templates were individually validated by testing in a sheared non-HPV human genomic DNA background matrix using the corresponding primer/probe as a single-probe assay, or as a primer/probe pool using the CHAMP-16 multi-probe assay. The number of copies detected with the individual target as template were comparable, when tested with the corresponding single-probe assay or with the CHAMP-16 multi-probe assay (Table 5). HPV16 copies detected by the CHAMP-16 assay using the pool of all 9 synthetic targets as template were, as anticipated, ~9-fold higher on average than the copies detected with a single target as template (Table 5).

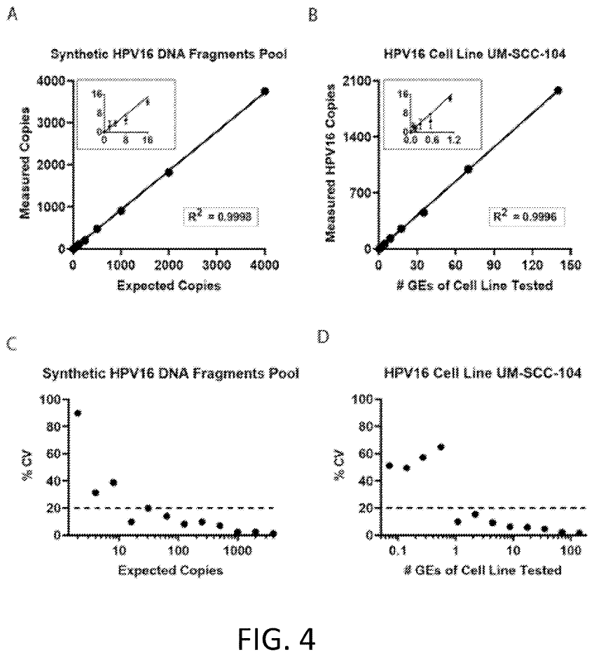

To determine the LoD of the CHAMP-16 assay, the synthetic pooled DNA template containing 9 targets or sheared UM-SCC-104 cell line gDNA template were spiked into hgen DNA matrix (~44,000 diploid GEs) and tested across different concentrations. Based on these serial dilution experiments, the LoD was determined to be <5 copies using both synthetic DNA pool and sheared UM-SCC-104 cell line gDNA templates (FIG. 4 Panels A and B). Since the CHAMP-16 assay targets 9 different regions of HPV16, these results indicate that the LoD of the assay is <1 GE of HPV16. The coefficient of variation (CV) was calculated at different template concentrations and determined to be lower than 20% for detection of 1 GE to 5 GEs, and lower than 10% for detection of >5 GEs, using synthetic HPV16 DNA pool or UM-SCC-104 gDNA (FIG. 4 Panels C and D).

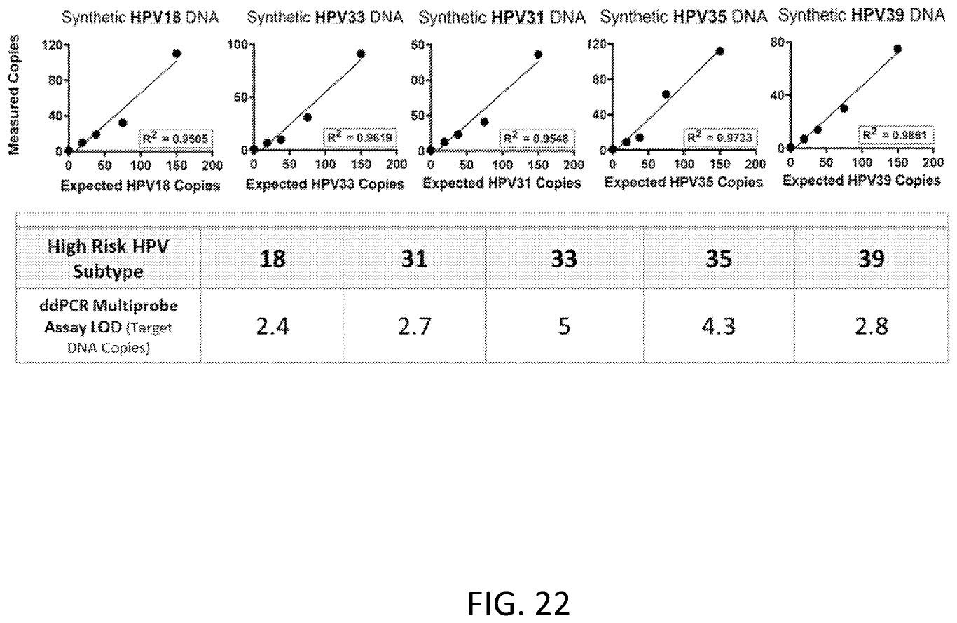

Using the analytical testing strategy for the CHAMP-16 assay as detailed above, analytical characteristics of a high-risk HPV pool, including the LoD for targeted high-risk HPV types, were defined. This demonstrated an analytical LoD ranging from 2.4 to 5 copies per mL plasma, depending on the specific HPV type (FIG. 22). A preliminary analysis showed that the high-risk multiprobe pool had an excellent signal to noise ratio and did not amplify non-HPV human genomic DNA and these primer/probe sets have been pooled to develop a CHAMP high risk (CHAMP-hr) HPV assay.

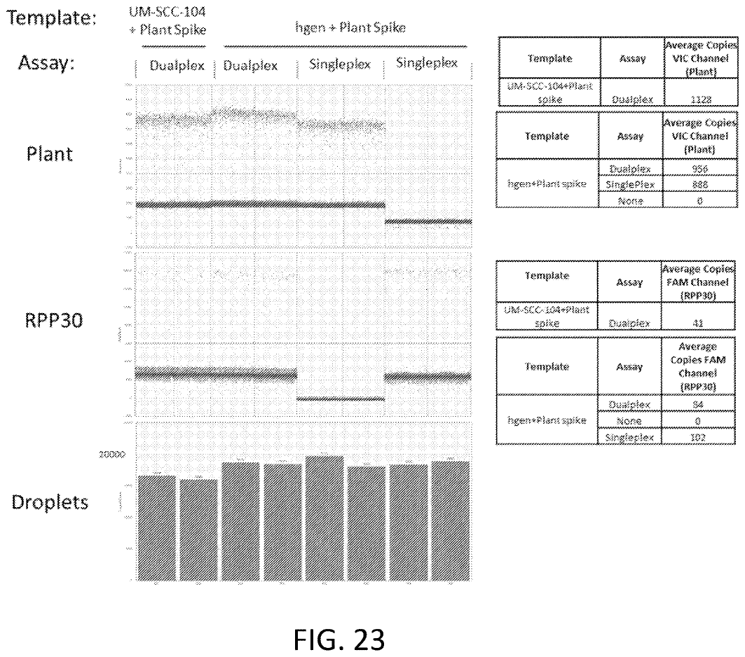

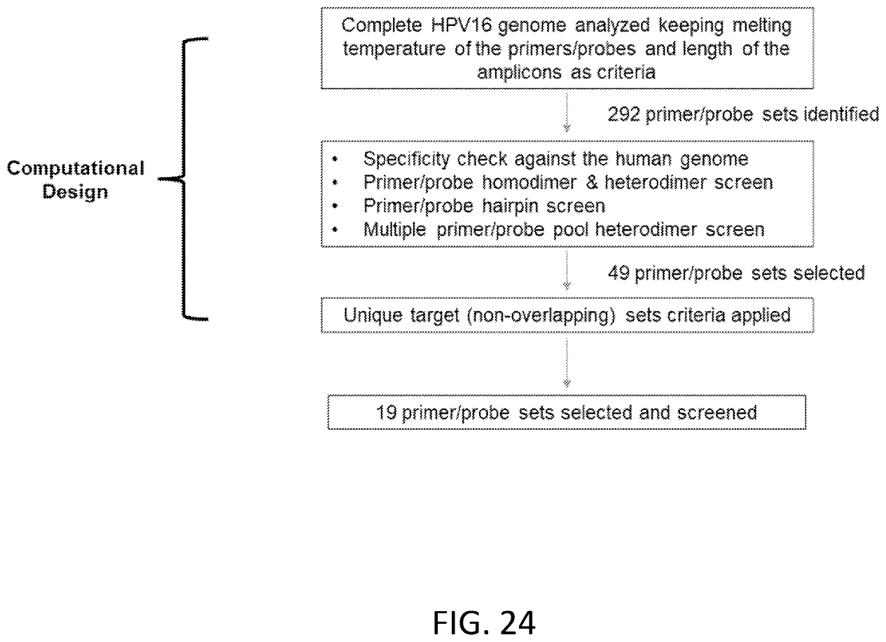

To control primary sample (patient specimen) processing and the molecular analysis of the analytes (processed sample), quality control measures were included. To control for sample processing, a synthetic 150 bp plant (Capsella bursa-pastoris) DNA, as a ‘technical control’ to normalize the recovery of plasma cfDNA extraction as described [54], was spiked in. Further, to control for analytes with signal below LoD and to assess the biological variation across samples, a widely used RPP30 reference gene assay for cfDNA analysis was included with the technical control as a dual-color pool assay, so that both quality control targets are quantified independently for each sample. Hence a dual-color assay was developed and was tested analytically for performance (FIG. 23).

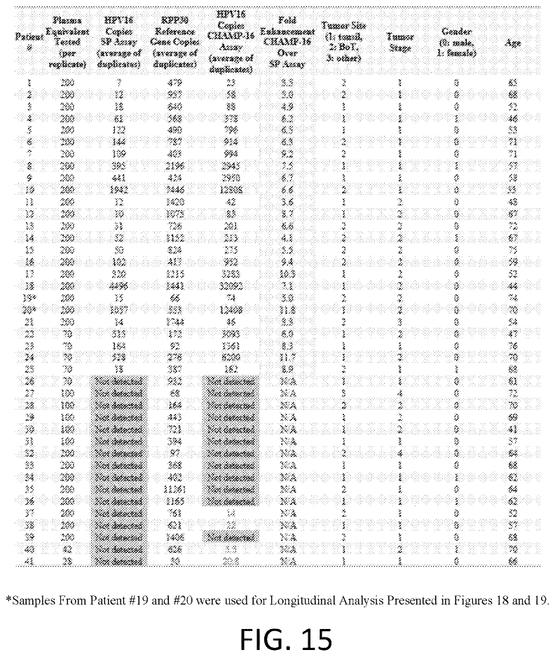

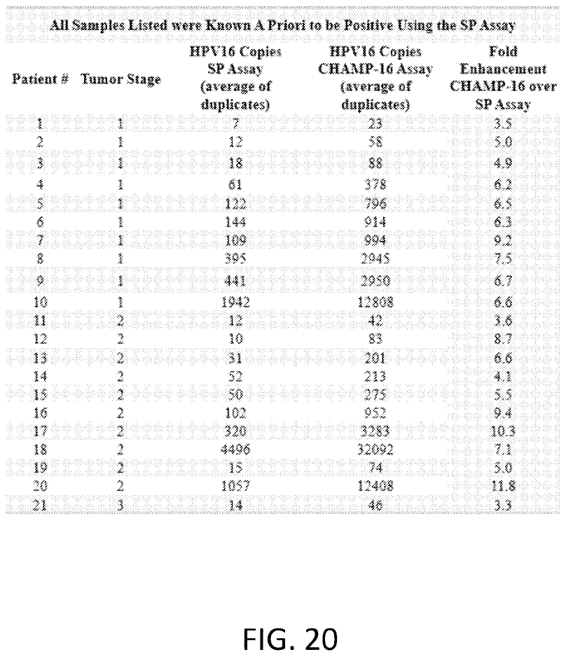

Testing of Clinical Plasma Specimens Using the CHAMP-16 Assay. Plasma cfDNA from 21 patients diagnosed with locally advanced HPV+ OPSCC who had detectable HPV16 ctDNA with the SP assay (FIG. 20 and FIG. 15) was used to compare the CHAMP-16 assay to the SP assay for detection of HPV16 ctDNA. The CHAMP-16 assay detected notably higher copies of HPV16 (6.6-fold on average) compared to the SP assay (FIG. 20). The CHAMP-16 assay was also highly specific for HPV as samples from patients with HPV negative cancers (n=11) and non-cancer control subjects (n=3) did not show any signal when tested (FIG. 16).

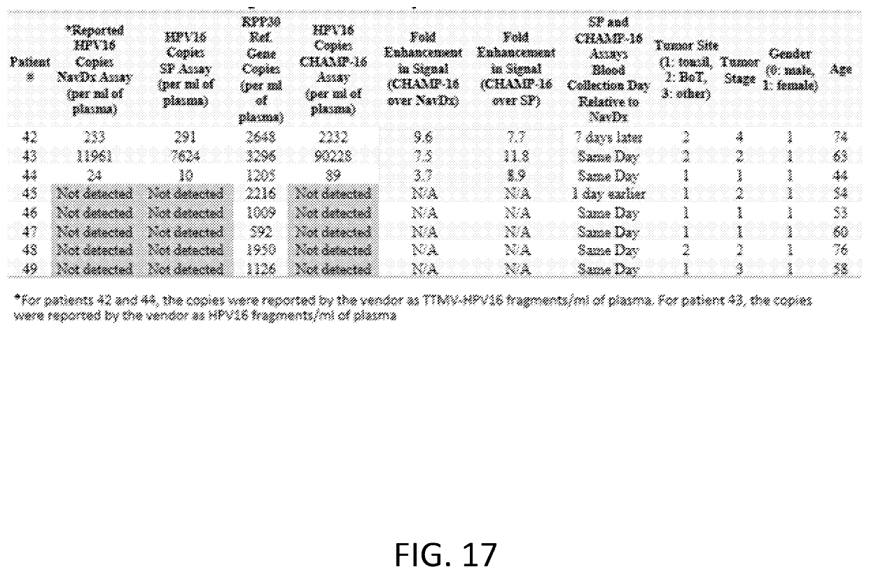

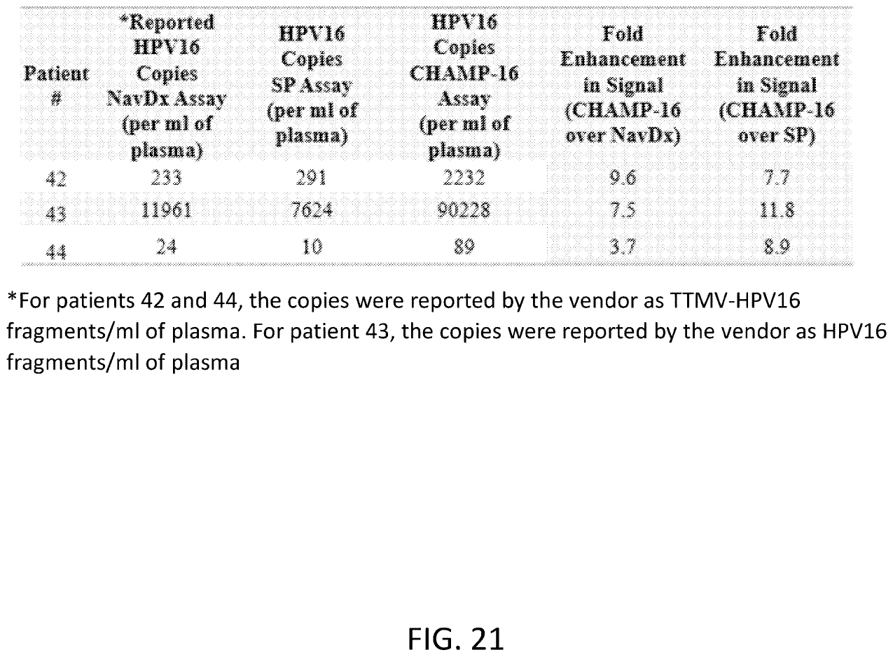

In addition to comparing the CHAMP-16 assay for clinical sample testing to the SP assay, it was also compared to a commercially available NavDx assay. HPV16 ctDNA copies were determined in plasma samples from 3 patients who had detectable HPV16 ctDNA levels reported at the same time point or close in time using the NavDx assay and from 5 patients who had been reported as negative with the NavDx assay. Consistent with the signal enhancement observed when compared to the SP assay, it was found that the CHAMP-16 assay displayed higher signal intensity (6.9-fold on average) when compared to the vendor reported values from NavDx testing in patients with detectable HPV16 ctDNA (FIG. 21 and FIG. 17), whereas no signal was observed with the 5 patients who had previously tested negative with NavDx assay (FIG. 17). Overall, the CHAMP-16 assay demonstrated a significantly higher signal detection of HPV16 ctDNA from plasma when compared to the SP assay or the tumor tissue modified viral (TTMV)-HPV DNA quantification reported by NavDx assay.

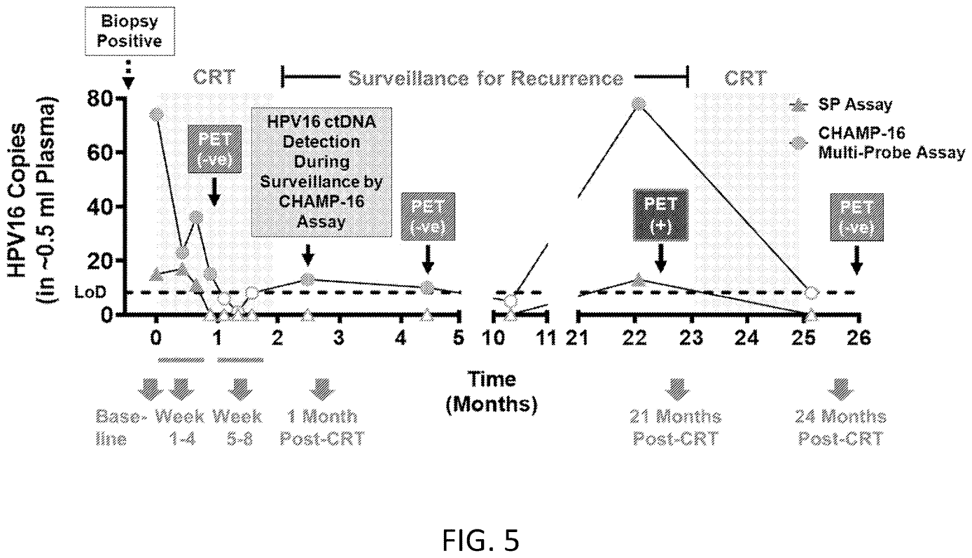

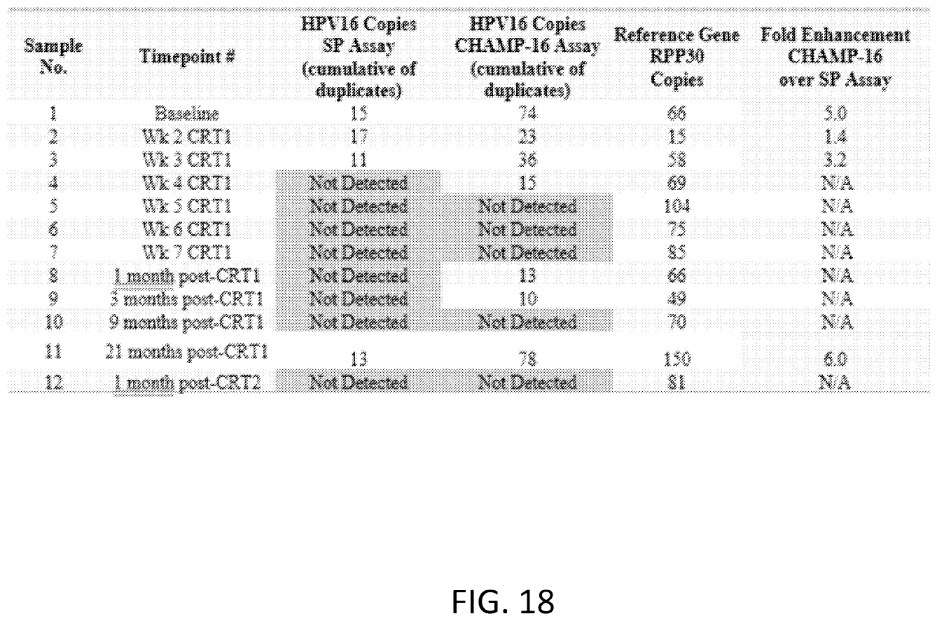

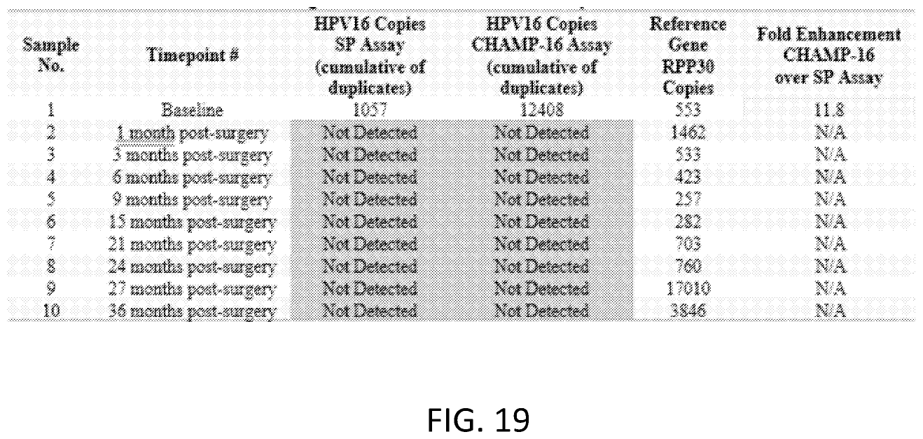

To gain insight into the clinical benefit of enhancement in signal detection using this assay, specimens were analyzed from a patient who underwent scheduled blood collections every three months after completion of definitive chemoradiation and developed recurrent disease (confirmed by PET/CT and subsequent biopsy). It was found that HPV16 ctDNA was detected by the CHAMP-16 assay 20 months prior to the clinical diagnosis, whereas the SP assay showed detectable HPV16 ctDNA only at the time point 1.5 weeks before the clinical diagnosis, among the available blood draw time points (FIG. 5 and FIG. 18). Longitudinally collected plasma samples were further tested from a patient with high ctDNA levels at baseline who did not clinically recur and found that the CHAMP-16 assay did not show any detectable signal in samples collected after the baseline sample (FIG. 19), in contrast to the patient who experienced cancer recurrence (FIG. 5 and FIG. 18). This highlights the value of the CHAMP-16 assay to provide increased signal and detect HPV16 ctDNA significantly earlier than the SP assay.

Finally, this assay was applied in an exploratory fashion to residual biobanked specimens from historical clinical trials (FIG. 15; patients 22 to 41) to evaluate its performance in detection of HPV16 ctDNA at baseline (i.e., prior to initiating treatment). Of note, these samples were collected and processed in previous studies differently than for the aforementioned 21 patient plasma samples (FIG. 20), including having undergone one or more freeze-thaw cycles. In addition, although these patients' tumor tissue was confirmed to p16(+), it was not universally known whether the tissues were positive for HPV16. Focusing on samples that tested positive using both the SP and CHAMP-16 assays (FIG. 15; patients 22 to 25), the CHAMP-16 assay showed an average of 8.7-fold signal enhancement when compared to the SP assay. An additional 4 samples from this cohort had detectable HPV16 ctDNA signal with the CHAMP-16 assay that was not detected by the SP assay (FIG. 15; patients 26 to 41). Together, these data indicate that the CHAMP-16 assay can increase the sensitivity of HPV16 detection and improve baseline detection over conventional single-probe assays.

HPV ctDNA analysis has had a rapid impact in the HPV-associated malignancy field. It is clear that HPV ctDNA testing can have a substantial long-term effect on the clinical decision-making paradigms for OPC. Past experience in biomarker assay development in oncology supports the importance of the availability of multiple assay options, for example as with multiple gene expression profile tests (MammaPrint, Oncotype Dx) for women with breast cancer [51,52]. The development of multiple assays for an important target such as HPV is critical because different clinical use cases may benefit from various optimized parameters. Given the sensitivity limitations of current HPV ctDNA LDTs, there is an established clinical need to develop higher sensitivity tests for the specific setting of detection of minimal residual disease [53].

Therefore, recognizing the need for additional HPV ctDNA ddPCR LDTs with comprehensively published assay details and analytical characteristics to enable benchmarking, as well as the need for future rigorous and confirmatory clinical studies to advance HPV ctDNA as a biomarker, a multi-probe HPV ctDNA assay was developed that has enhanced analytical sensitivity relative to the SP assay and 6.9× signal enhancement over a commercial assay (NavDx) in head-to-head testing using a limited set of available collection time-matched samples. Accordingly, this enhancement in signal yields an assay with a LOD of <1 GE, which is comparable to what was observed in an ultra-sensitive HPV-seq study using next generation sequencing [53]. It was also noted that reporting metrics of the NavDx assay have recently changed. Consequently, the current limitation of available samples to benchmark performance against other assays demonstrates the urgent need in this field to build a shared repository of HPV+plasma samples, especially considering the multiple HPV ctDNA assays that are currently in development by both academic and commercial entities.

Overall, this high performance ddPCR approach continues to be considerably less expensive, faster, and simpler than NGS approaches. Thus, a lower cost ddPCR-based approach developed here has advantages for early detection and/or surveillance screening where more frequent testing is beneficial to patients.

### Example 5

TABLE 10
Computational Screens. The primers and probes were designed using the
HPV16 sequence (NC_001562.4 - 7906 bp). HPV16 genomic sequence was
scanned using the PrimerQuest tool from Integrated DNA Technologies
with the following parameters using the qPCR (2 Primers + Probe) option:
(a) reaction conditions

| | |
|---|---|
| Monovalent Salt (Na⁺) (mM) | 50* |
| Each Primer DNA Concentration (nM) | 900 |
| Divalent Salt (Mg⁺⁺) (mM) | 5* |
| Probe DNA Concentration (nM) | 250 |
| dNTP Concentration (mM) | 0.8* |

(b) primer criteria

| | |
|---|---|
| Primer Tm (°C) | Minimum: 57; Optimal: 62; Maximum: 68 |
| Primer GC (%) | Minimum: 35; Optimal: 50; Maximum: 65 |
| Primer Size (bp) | Minimum: 17; Optimal: 22; Maximum: 28 |

(c) probe criteria

| | |
|---|---|
| FProbe Tm (°C) | Minimum: 59; Optimal: 63; Maximum: 70 |
| Probe GC (%) | Minimum: 40; Optimal: 50; Maximum: 67 |
| Probe Size (bp) | Minimum: 20; Optimal: 25; Maximum: 30 |

(d) amplicon criteria

| | |
|---|---|
| Amplicon Size (bp) | Minimum: 65; Optimal: 80; Maximum: 100 |

*Concentration in the ddPCR Supermix (#186-3024, Bio-Rad) is proprietary, and the value is being reported as an estimate based on personal communication

A maximum of 50 results were returned with each search using the PrimerQuest tool. To avoid saturation, a 0.8-1 kb window of the 7906 bp HPV16 sequence was scanned keeping a 200 bp overlap at the 5′ and 3′ end. There were a total of 321 primer and probe (primer/probe) combinations (963 oligonucleotide sequences) identified using the above criteria in the PrimerQuest tool across the entire HPV16 genome. After removing the redundant primer/probe combinations in every 200 bp overlapping sequence between different 0.8-1 kb batches, there were a total of 292 unique combinations (876 oligonucleotide sequences) identified as candidates for further screens.

The selected 292 combinations were checked for homology with the human genome (Taxonomy ID: 9606) using NCBI BLASTn megablast program. Any primer or probe with homology greater than 15 bp to an annotated region was rejected. This criterion led to a selection of 67 combinations (201 oligonucleotide sequences).

Since the primers and probes were being designed to be pooled in a single reaction and detect multiple regions of HPV16 from a cfDNA sample, the selected 67 primer/probe combinations were checked for heterodimerization with the other primers or probes using the Primer Tools program from National Institute of Standards and Technology. A maximum of 100 batched sequences could be screened using this tool. Hence the primer/probe combinations were screened in batches of 33 combinations (99 oligonucleotide sequences) in multiple iterations and heterodimers with greater than 8 bp homology (continuous) and a melting temperature greater than 20° C. for the homologous region were rejected. Following the heterodimer screen, the primers and probes were screened for self-annealing as hairpins or for homodimer formation, using the same Primer Tools program and the PCR Primer Stats program from Genscript, respectively. Primers or probes that were not predicted to have greater than 8 bp stretch of bases annealing as a homodimer, hairpin or heterodimer were selected, leading to a total of 49 final primer/probe combinations (147 oligonucleotide sequences).

TABLE 11
Final PCR Conditions for CHAMP-16 Assay, CHAMP-hr
Assay, and RPP-30/Plant Spike Dual Assay.

| | | | |
|---|---|---|---|
| Temp | Time | Ramp Rate | Cycles |

| | | | | | |
|---|---|---|---|---|---|
| 95 | °C | 10 | min | 1 °C/sec | 1 |
| 94 | °C | 30 | sec | 1 °C/sec | 50 |
| 59 | °C | 2 | min | 1 °C/sec | |
| 98 | °C | 10 | min | 1 °C/sec | 1 |

| | | | | |
|---|---|---|---|---|
| 4 | °C | Hold | 1 °C/sec | 1 |

TABLE 12
38 × CHAMP-16 Assay Mix.

| | Dilution Factor | Final Molarity | Each Primer | Total Volume |
|---|---|---|---|---|
| | to Make 38 × Stock | in 38 × Stock | or Probe | with 9 Primer/ |
| Component | from 500 μM | (μM) | Volume (μl) | Probe sets (μl) |
| 500 μM | 30.9 | 16.2 | 2 | 18 |
| F Primer | | | | |
| 500 μM | 30.9 | 16.2 | 2 | 18 |
| R Primer | | | | |
| 100 μM | 22.2 | 4.5 | 2.8 | 25.2 |
| Probe | | | | |
| IDTE | | | | 0.5 |
| Buffer | | | | |
| Total | | | | 61.7 |

TABLE 13
38 × CHAMP-hr Assay Mix.

| | Dilution Factor | Final Molarity | Each Primer | Total Volume |
|---|---|---|---|---|
| | to Make 38 × Stock | in 38 × Stock | or Probe | with 5 Primer/ |
| Component | from 500 μM | (μM) | Volume (μl) | Probe sets (μl) |
| 500 μM F | 30.9 | 16.2 | 2 | 10 |
| Primer | | | | |
| 500 μM R | 30.9 | 16.2 | 2 | 10 |
| Primer | | | | |
| 100 μM | 22.2 | 4.5 | 2.8 | 14 |
| Probe | | | | |
| IDTE | | | | 27.7 |
| Buffer | | | | |
| Total | | | | 61.7 |

TABLE 14
38 × (SP Assay + RPP30 Reference Gene Assay) Mix.

| | Dilution Factor | Final Molarity | Each Primer | Total Volume |
|---|---|---|---|---|
| | to Make 38 × Stock | in 38 × Stock | or Probe | with 2 Primer/ |
| Component | from 500 μM | (μM) | Volume (μl) | Probe sets (μl) |
| 500 μM F | 15.45 | 32.4 | 4 | 8 |
| Primer | | | | |
| 500 μM R | 15.45 | 32.4 | 4 | 8 |
| Primer | | | | |
| 100 μM | 11.1 | 9.1 | 5.6 | 11.2 |
| Probe | | | | |
| IDTE | | | | 34.5 |
| Buffer | | | | |
| Total | | | | 61.7 |

TABLE 15
38 × (RPP30 Reference Gene Assay + Plant Spike Assay) Mix.

| | Dilution Factor | Final Molarity | Each Primer | Total Volume |
|---|---|---|---|---|
| | to Make 38 × Stock | in 38 × Stock | or Probe | with 2 Primer/ |
| Component | from 500 μM | (μM) | Volume (μl) | Probe sets (μl) |
| 500 μM F | 15.45 | 32.4 | 4 | 8 |
| Primer | | | | |
| 500 μM R | 15.45 | 32.4 | 4 | 8 |
| Primer | | | | |
| 100 μM | 11.1 | 9.1 | 5.6 | 11.2 |
| Probe | | | | |
| IDTE | | | | 34.5 |
| Buffer | | | | |
| Total | | | | 61.7 |

TABLE 16
Droplet Generation Set-up for CHAMP-16 Assay.

| | Volume per |
|---|---|
| Component | Reaction (μl) |
| Template | Up to 10.19 |
| 2 × ddPCR Supermix | 10.75 |
| (No dUTP) | |
| 38 × CHAMP-16 Assay Mix | 0.56 |
| Total | 21.50 |
| | 20 μl used for |
| | droplet generation |

TABLE 17
Droplet Generation Set-up for CHAMP-hr Assay.

| | Volume per |
|---|---|
| Component | Reaction (μl) |
| Template | Up to 10.19 |
| 2 × ddPCR Supermix | 10.75 |
| (No dUTP) | |
| 38 × CHAMP-hr Assay Mix | 0.56 |
| Total | 21.50 |
| | 20 μl used for |
| | droplet generation |

TABLE 18
Droplet Generation Set-up for SP Assay +
RPP30 Reference Gene Assay.

| | Volume per |
|---|---|
| Component | Reaction (μl) |
| Template | Up to 10.19 |
| 2 × ddPCR Supermix | 10.75 |
| (No dUTP) | |
| 38 × (SP Assay + RPP30 | 0.56 |
| Reference Gene Assay) Mix | |
| Total | 21.50 |
| | 20 μl used for |
| | droplet generation |

TABLE 19
Droplet Generation Set-up for RPP30 Reference
Gene Assay + Plant Spike Assay.

| | Volume per |
|---|---|
| Component | Reaction (μl) |
| Template | Up to 10.19 |
| 2 × ddPCR Supermix | 10.75 |
| (No dUTP) | |
| 38 × (RPP30 Reference Gene | 0.56 |
| Assay + Plant Spike Assay) Mix | |
| Total | 21.50 |
| | 20 μl used for |
| | droplet generation |

### REFERENCES

[1] Ramqvist T, Dalianis T. Oropharyngeal cancer epidemic and human papillomavirus. Emerg Infect Dis 2010; 16:1671-7.
[2] Ramqvist T, Dalianis T. An epidemic of oropharyngeal squamous cell carcinoma (OSCC) due to human papillomavirus (HPV) infection and aspects of treatment and prevention. Anticancer Res 2011; 31:1515-9.
[3] Chaturvedi A K, Engels E A, Pfeiffer R M, Hernandez B Y, Xiao W, Kim E, et al. Human papillomavirus and rising oropharyngeal cancer incidence in the United States. J Clin Oncol 2011; 29:4294-301.
[4] Marur S, D'Souza G, Westra W H, Forastiere A A. HPV-associated head and neck cancer: a virus-related cancer epidemic. Lancet Oncol 2010; 11:781-9.
[5] Attner P, Du J, Näsman A, Hammarstedt L, Ramqvist T, Lindholm J, et al. The role of human papillomavirus in the increased incidence of base of tongue cancer. Int J Cancer 2010; 126:2879-84.
[6] Chaturvedi A K, Engels E A, Anderson W F, Gillison M L. Incidence trends for human papillomavirus-related and-unrelated oral squamous cell carcinomas in the United States. J Clin Oncol 2008; 26:612-9.
[7] Saraiya M, Unger E R, Thompson T D, Lynch C F, Hernandez B Y, Lyu C W, et al. US assessment of HPV types in cancers: implications for current and 9-valent HPV vaccines. J Natl Cancer Inst 2015; 107: djv086.
[8] Mellin H, Friesland S, Lewensohn R, Dalianis T, Munck-Wikland E. Human papillomavirus (HPV) DNA in tonsillar cancer: clinical correlates, risk of relapse, and survival. Int J Cancer 2000; 89:300-4.
[9] Chen M M, Mott N, Clark S J, Harper D M, Shuman A G, Prince M E P, et al. HPV Vaccination Among Young Adults in the US. JAMA 2021; 325:1673-4.
[10] Ang K K, Harris J, Wheeler R, Weber R, Rosenthal D I, Nguyen-Tân P F, et al. Human papillomavirus and survival of patients with oropharyngeal cancer. N Engl J Med 2010; 363:24-35.
[11] Grønhøj C, Jakobsen K K, Jensen D H, Rasmussen J, Andersen E, Friborg J, et al. Pattern of and survival following loco-regional and distant recurrence in patients with HPV+ and HPV-oropharyngeal squamous cell carcinoma: A population-based study. Oral Oncol 2018; 83:127-33.
[12] Trosman S J, Koyfman S A, Ward M C, Al-Khudari S, Nwizu T, Greskovich J F, et al. Effect of human papillomavirus on patterns of distant metastatic failure in oropharyngeal squamous cell carcinoma treated with chemoradiotherapy. JAMA Otolaryngol Head Neck Surg 2015; 141:457-62.
[13] Leeman J E, Li J G, Pei X, Venigalla P, Zumsteg Z S, Katsoulakis E, et al. Patterns of Treatment Failure and Postrecurrence Outcomes Among Patients With Locally Advanced Head and Neck Squamous Cell Carcinoma After Chemoradiotherapy Using Modern Radiation Techniques. JAMA Oncol 2017; 3:1487-94.
[14] Smerage J B, Barlow W E, Hortobagyi G N, Winer E P, Leyland-Jones B, Srkalovic G, et al. Circulating tumor cells and response to chemotherapy in metastatic breast cancer: SWOG S0500. J Clin Oncol 2014; 32:3483-9.
[15] Kunath F, Jensen K, Pinart M, Kahlmeyer A, Schmidt S, Price C L, et al. Early versus deferred standard androgen suppression therapy for advanced hormone-sensitive prostate cancer. Cochrane Database Syst Rev 2019; 6: CD003506.
[16] Buyyounouski M K, Pickles T, Kestin L L, Allison R, Williams S G. Validating the interval to biochemical failure for the identification of potentially lethal prostate cancer. J Clin Oncol 2012; 30:1857-63.
[17] Radovich M, Jiang G, Hancock B A, Chitambar C, Nanda R, Falkson C, et al. Association of Circulating Tumor DNA and Circulating Tumor Cells After Neoadjuvant Chemotherapy With Disease Recurrence in Patients With Triple-Negative Breast Cancer: Preplanned Secondary Analysis of the BRE12-158 Randomized Clinical Trial. JAMA Oncol 2020; 6:1410-5.
[18] Sparano J, O'Neill A, Alpaugh K, Wolff A C, Northfelt D W, Dang C T, et al. Association of Circulating Tumor Cells With Late Recurrence of Estrogen Receptor-Positive Breast Cancer: A Secondary Analysis of a Randomized Clinical Trial. JAMA Oncol 2018; 4:1700-6.
[19] Garcia-Murillas I, Chopra N, Comino-Méndez I, Beaney M, Tovey H, Cutts R J, et al. Assessment of Molecular Relapse Detection in Early-Stage Breast Cancer. JAMA Oncol 2019; 5:1473-8.
[20] Patel S N, Cohen M A, Givi B, Dixon B J, Gilbert R W, Gullane P J, et al. Salvage surgery for locally recurrent oropharyngeal cancer. Head Neck 2016; 38 Suppl 1: E658-64.
[21] Nair V, Auger S, Kochanny S, Howard F M, Ginat D, Pasternak-Wise O, et al. Development and Validation of a Decision Analytical Model for Posttreatment Surveillance for Patients With Oropharyngeal Carcinoma. JAMA Netw Open 2022; 5: e227240.
[22] Ho A S, Tsao G J, Chen F W, Shen T, Kaplan M J, Colevas A D, et al. Impact of positron emission tomography/computed tomography surveillance at 12 and 24 months for detecting head and neck cancer recurrence. Cancer 2013; 119:1349-56.
[23] Swiecicki P L, Brennan J R, Mierzwa M, Spector M E, Brenner J C. Head and Neck Squamous Cell Carcinoma Detection and Surveillance: Advances of Liquid Biomarkers. Laryngoscope 2019; 129:1836-43.
[24] Chatfield-Reed K, Roche V P, Pan Q. cfDNA detection for HPV+ squamous cell carcinomas. Oral Oncol 2021; 115:104958.
[25] Dermody S M, Haring C T, Bhambhani C, Tewari M, Brenner J C, Swiecicki P L. Surveillance and Monitoring Techniques for HPV-Related Head and Neck Squamous Cell Carcinoma: Circulating Tumor DNA. Curr Treat Options Oncol 2021; 22:21.
[26] Kong L, Birkeland A C. Liquid Biopsies in Head and Neck Cancer: Current State and Future Challenges. Cancers 2021; 13. https://doi.org/10.3390/cancers13081874.
[27] Mishra V, Singh A, Chen X, Rosenberg A J, Pearson A T, Zhavoronkov A, et al. Application of liquid biopsy as multi-functional biomarkers in head and neck cancer. Br J Cancer 2022; 126:361-70.
[28] Colevas A D. HPV DNA as a Biomarker in Oropharyngeal Cancer: A Step in the Right Direction. Clin Cancer Res 2022; 28:4171-2.
[29] Haring C T, Bhambhani C, Brummel C, Jewell B, Bellile E, Heft Neal M E, et al. Human papilloma virus circulating tumor DNA assay predicts treatment response in recurrent/metastatic head and neck squamous cell carcinoma. Oncotarget 2021; 12:1214-29.
[30] Haring C T, Brummel C, Bhambhani C, Jewell B, Neal M H, Bhangale A, et al. Implementation of human papillomavirus circulating tumor DNA to identify recurrence during treatment de-escalation. Oral Oncol 2021; 121:105332.
[31] Cao Y, Haring C T, Brummel C, Bhambhani C, Aryal M, Lee C, et al. Early HPV ctDNA Kinetics and Imaging Biomarkers Predict Therapeutic Response in p16+ Oropharyngeal Squamous Cell Carcinoma. Clin Cancer Res 2022; 28:350-9.
[32] Berger B M, Hanna G J, Posner M R, Genden E M, Lautersztain J, Naber S P, et al. Detection of Occult Recurrence Using Circulating Tumor Tissue Modified Viral HPV DNA among Patients Treated for HPV-Driven Oropharyngeal Carcinoma. Clin Cancer Res 2022; 28:4292-301.
[33] Chera B S, Kumar S, Shen C, Amdur R, Dagan R, Green R, et al. Plasma Circulating Tumor HPV DNA for the Surveillance of Cancer Recurrence in HPV-Associated Oropharyngeal Cancer. J Clin Oncol 2020; 38:1050-8.
[34] Chera B S, Kumar S, Beaty B T, Marron D, Jefferys S, Green R, et al. Rapid Clearance Profile of Plasma Circulating Tumor HPV Type 16 DNA during Chemoradiotherapy Correlates with Disease Control in HPV-Associated Oropharyngeal Cancer. Clin Cancer Res 2019; 25:4682-90.
[35] Alcaide M, Cheung M, Hillman J, Rassekh S R, Deyell R J, Batist G, et al. Evaluating the quantity, quality and size distribution of cell-free DNA by multiplex droplet digital PCR. Sci Rep 2020; 10:12564.
[36] PrimerQuest-design qPCR assays. Integrated DNA Technologies n.d. https://www.idtdna.com/pages/tools/primerquest (accessed Feb. 1, 2023).
[37] Johnson M, Zaretskaya I, Raytselis Y, Merezhuk Y, McGinnis S, Madden T L. NCBI BLAST: a better web interface. Nucleic Acids Res 2008; 36: W5-9.
[38] Vallone P M, Butler J M. AutoDimer: a screening tool for primer-dimer and hairpin structures. Biotechniques 2004; 37:226-31.
[39] Stothard P. The sequence manipulation suite: JavaScript programs for analyzing and formatting protein and DNA sequences. Biotechniques 2000; 28:1102, 1104.
[40] Robins H S, Ericson N G, Guenthoer J, O'Briant K C, Tewari M, Drescher C W, et al. Digital genomic quantification of tumor-infiltrating lymphocytes. Sci Transl Med 2013; 5:214ra169.
[41] Witte A K, Mester P, Fister S, Witte M, Schoder D, Rossmanith P. A Systematic Investigation of Parameters Influencing Droplet Rain in the Listeria monocytogenes prfA Assay-Reduction of Ambiguous Results in ddPCR. PLOS One 2016; 11: e0168179.
[42] Gerdes L, Iwobi A, Busch U, Pecoraro S. Optimization of digital droplet polymerase chain reaction for quantification of genetically modified organisms. Biomol Detect Quantif 2016; 7:9-20.
[43] Hindson C M, Chevillet J R, Briggs H A, Gallichotte E N, Ruf I K, Hindson B J, et al. Absolute quantification by droplet digital PCR versus analog real-time PCR. Nat Methods 2013; 10:1003-5.
[44] Armbruster D A, Pry T. Limit of blank, limit of detection and limit of quantitation. Clin Biochem Rev 2008; 29 Suppl 1: S49-52.
[45] Akagi K, Li J, Broutian T R, Padilla-Nash H, Xiao W, Jiang B, et al. Genome-wide analysis of HPV integration in human cancers reveals recurrent, focal genomic instability. Genome Res 2014; 24:185-99.
[46] Aucamp J, Bronkhorst A J, Badenhorst C P S, Pretorius P J. The diverse origins of circulating cell-free DNA in the human body: a critical re-evaluation of the literature. Biol Rev Camb Philos Soc 2018; 93:1649-83.
[47] Jiang P, Chan C W M, Chan K C A, Cheng S H, Wong J, Wong V W S, et al. Lengthening and shortening of plasma DNA in hepatocellular carcinoma patients. Proc Natl Acad Sci US A 2015; 112: E1317-25.
[48] Begum S, Gillison M L, Nicol T L, Westra W H. Detection of human papillomavirus-16 in fine-needle aspirates to determine tumor origin in patients with metastatic squamous cell carcinoma of the head and neck. Clin Cancer Res 2007; 13:1186-91.
[49] Ruzevick J, Olivi A, Westra W H. Metastatic squamous cell carcinoma to the brain: an unrecognized pattern of distant spread in patients with HPV-related head and neck cancer. J Neurooncol 2013; 112:449-54.
[50] Vandenberg O, Martiny D, Rochas O, van Belkum A, Kozlakidis Z. Considerations for diagnostic COVID-19 tests. Nat Rev Microbiol 2021; 19:171-83.
[51] Carlson J J, Roth J A. The impact of the Oncotype Dx breast cancer assay in clinical practice: a systematic review and meta-analysis. Breast Cancer Res Treat 2013; 141:13-22.
[52] Wittner B S, Sgroi D C, Ryan P D, Bruinsma T J, Glas A M, Male A, et al. Analysis of the MammaPrint breast cancer assay in a predominantly postmenopausal cohort. Clin Cancer Res 2008; 14:2988-93.
[53] Leung E, Han K, Zou J, Zhao Z, Zheng Y, Wang T T, et al. HPV Sequencing Facilitates Ultrasensitive Detection of HPV Circulating Tumor DNA. Clin Cancer Res 2021; 27:5857-68.

What is claimed is:

1. A composition comprising one or more oligonucleotides that detect a one or more target regions selected from the group consisting of: SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36; SEQ ID NO: 52; SEQ ID NO: 53; SEQ ID NO: 54; SEQ ID NO: 56.

2. The composition of claim 1, wherein the one or more oligonucleotides are selected from the group consisting of: SEQ ID NOs: 1-27 and SEQ ID NOs: 37-51.

3. The composition of claim 1 or 2, wherein at least a triad of the one or more oligonucleotides are selected from the group consisting of:
Set 1: SEQ ID NO: 1, SEQ ID NO: 2, SEQ ID NO: 3;
Set 2: SEQ ID NO: 4, SEQ ID NO: 5, SEQ ID NO: 6;
Set 3: SEQ ID NO: 7, SEQ ID NO: 8, SEQ ID NO: 9;
Set 4: SEQ ID NO: 10, SEQ ID NO: 11, SEQ ID NO: 12;
Set 5: SEQ ID NO: 13, SEQ ID NO: 14, SEQ ID NO: 15;
Set 6: SEQ ID NO: 16, SEQ ID NO: 17, SEQ ID NO: 18;
Set 7: SEQ ID NO: 19, SEQ ID NO: 20, SEQ ID NO: 21;
Set 8: SEQ ID NO: 22, SEQ ID NO: 23, SEQ ID NO: 24;
Set 9: SEQ ID NO: 25, SEQ ID NO: 26, SEQ ID NO: 27;
Set 10: SEQ ID NO: 37, SEQ ID NO: 38, SEQ ID NO: 39;
Set 11: SEQ ID NO: 40, SEQ ID NO: 41, SEQ ID NO: 42;
Set 12: SEQ ID NO: 43, SEQ ID NO: 44, SEQ ID NO: 45;
Set 13: SEQ ID NO: 46, SEQ ID NO: 47, SEQ ID NO: 48;
Set 14: SEQ ID NO: 49, SEQ ID NO: 50, SEQ ID NO: 51.

4. The composition of claim 1 or 2, wherein the one or more oligonucleotides is selected from the group consisting of:
a forward primer having at least 90% sequence identity to any one or more of: SEQ ID NOs: 1, 4, 7, 10, 13, 16, 19, 22, 25, 37, 40, 43, 46, 49;
a reverse primer having at least 90% sequence identity to any one or more of: SEQ ID NOs: 2, 5, 8, 11, 14, 17, 20, 23, 26, 39, 42, 45, 48, 51;
a probe comprising a detectable label, wherein the forward primer and the reverse primer anneal to a target HPV sequence.

5. The composition of claim 4, wherein said probe has at least 90% sequence identity to any one or more of: SEQ ID NOs: 3, 6, 9, 12, 15, 18, 21, 24, 27, 38, 41, 44. 47, 50.

6. The composition of any one of claims 1-5, wherein a first set of oligonucleotides comprises SEQ ID NOs: 1-15 or SEQ ID NOs: 37-45.

7. The composition of any one of claims 1-6, wherein a second set of oligonucleotides comprises SEQ ID NOs: 16-27 or SEQ ID NOs: 46-51.

8. The composition of any one of claims 1-7, wherein the one or more oligonucleotides do not have greater than an eight-base pair stretch of bases annealing as a homodimer, hairpin, or heterodimer.

9. The composition of any one of claims 1-8 wherein a first set of oligonucleotides comprises a first detectable label and a second set of oligonucleotides comprises a second detectable label.

10. The composition of claim 9, wherein said second labeled set of oligonucleotides targets a viral E6/E7 junction and wherein said junction is highly represented in HPV ctDNA.

11. The composition of any one of claims 9-10, wherein said second labeled set of oligonucleotides is selected from the group with SEQ ID Nos: 2, 5, 8, 11, 14, 17, 20, 23, 26, 39, 42, 45, 48, 51 and said first labeled set of oligonucleotides is selected from the group with SEQ ID Nos: SEQ ID NOs: 1, 4, 7, 10, 13, 16, 19, 22, 25, 37, 40, 43, 46, 49.

12. A method comprising contacting a sample with a composition of any one of claims 1-11.

13. The method of claim 12, wherein the sample is blood.

14. The method of claim 12, wherein the sample is derived from blood.

15. The method of claim 12, wherein the sample is amniotic fluid, ascites, bile, breast milk, breast milk colostrum, bronchoalveolar lavage fluid, cerebrospinal fluid, dialysate, eye aqueous humor, eye vitreous humor, feces, paracentesis, pericardial fluid, peritoneal, blood plasma, pleural, semen, blood serum, synovial fluid, tears, thoracentesis, blood, saliva, gargle, or urine.

16. The method of claim 12, wherein the contacting comprises: (i) providing one or more of the oligonucleotides; (ii) fractionating a plurality of HPV DNA from the sample into droplets at a concentration wherein, on average, only 0 or 1 molecule of the DNA is present in each droplet; (iii) amplifying HPV DNA in each droplet with the one or more oligonucleotides to produce amplicon signals; and (iv) detecting in each droplet any amplicon signals.

17. The method of claim 16, wherein DNA is fractionated into micro-droplets by emulsification.

18. The method of claim 16 or 17, wherein DNA is amplified using a nucleic acid amplification method.

19. The method of claim 12, wherein the contacting comprises conducting a quantitative PCR (qPCR) assay.

20. The method of claim 19, wherein the qPCR assay comprises a digital PCR assay.

21. The method of claim 20, wherein the digital PCR assay comprises a droplet digital PCR (ddPCR) assay.

22. The method of claim 12, wherein the sample comprises a ctDNA HPV target nucleic acid and wherein one or more of the oligonucleotides hybridize to the HPV target nucleic acid.

23. The method of claim 12, further comprising a step of treating a subject, if HPV is detected in the sample, with cancer surveillance, therapeutic, or intervention.

24. The method of claim 23, wherein the step of treating a subject is provided prior to the appearance of CT scan-detectable disease.

25. The method of claim 12, further comprising a step of treating a subject, if HPV is not detected in the sample, with a HPV vaccine.

26. The method of claim 12, further comprising sequencing HPV nucleic acid present in the sample.

27. A kit comprising a composition of any one of claims 1-12 and reagents to purify, isolate, detect, and/or quantify HPV ctDNA.

28. The kit of claim 26; wherein said reagents comprise: (i) amplification reagents; (ii) control samples; (iii) solid surfaces comprising capture reagents specific for target ctDNA;
(iv) containers for holding or storing a sample; (v) one or more instruments for assisting with obtaining a test sample; (vi) reaction vessels, mixing vessels, and (vii) instructions for use of the kit.


SEQUENCE LISTING (183 sequences)

SEQ ID NO: 1 - DNA, 21 bp, synthetic construct
  source 1..21: mol_type = other DNA
ggaacaacattagaacagcaa

SEQ ID NO: 2 - DNA, 18 bp, synthetic construct
  source 1..18: mol_type = other DNA
ttcttcaggacacagtgg

SEQ ID NO: 3 - DNA, 22 bp, synthetic construct
  source 1..22: mol_type = other DNA
caacaaaccgttgtgtgatttg

SEQ ID NO: 4 - DNA, 20 bp, synthetic construct
  source 1..20: mol_type = other DNA
gagagggtgttagtgaaaga

SEQ ID NO: 5 - DNA, 20 bp, synthetic construct
  source 1..20: mol_type = other DNA
gctaacattgctgcctttgc

SEQ ID NO: 6 - DNA, 24 bp, synthetic construct
  source 1..24: mol_type = other DNA
cactatatgccaaacaccacttac

SEQ ID NO: 7 - DNA, 21 bp, synthetic construct
  source 1..21: mol_type = other DNA
gtaataaatcaacgtgttgcg

SEQ ID NO: 8 - DNA, 20 bp, synthetic construct
  source 1..20: mol_type = other DNA
gtcagctatactgggtgtaa

SEQ ID NO: 9 - DNA, 20 bp, synthetic construct
  source 1..20: mol_type = other DNA
tggtgtattgctgcatttgg

SEQ ID NO: 10 - DNA, 19 bp, synthetic construct
  source 1..19: mol_type = other DNA
ggcattagtggccatcctt

SEQ ID NO: 11 - DNA, 23 bp, synthetic construct
  source 1..23: mol_type = other DNA
ctattatccacacctgcatttgc

SEQ ID NO: 12 - DNA, 23 bp, synthetic construct
  source 1..23: mol_type = other DNA
aaatgctagtgcttatgcagcaa

SEQ ID NO: 13 - DNA, 22 bp, synthetic construct
  source 1..22: mol_type = other DNA
agttctatgtcagcaactatgg

SEQ ID NO: 14 - DNA, 17 bp, synthetic construct
  source 1..17: mol_type = other DNA
cagtgcaggtcaggaaa

SEQ ID NO: 15 - DNA, 20 bp, synthetic construct
  source 1..20: mol_type = other DNA
cttgtacgtttcctgcttgc

SEQ ID NO: 16 - DNA, 22 bp, synthetic construct
  source 1..22: mol_type = other DNA
ctaacattaatgctggtacaga

SEQ ID NO: 17 - DNA, 21 bp, synthetic construct
  source 1..21: mol_type = other DNA
gaaatgtaaacaccaccaatc

SEQ ID NO: 18 - DNA, 20 bp, synthetic construct
  source 1..20: mol_type = other DNA
tctaggtggccttatttaca

SEQ ID NO: 19 - DNA, 19 bp, synthetic construct
  source 1..19: mol_type = other DNA
agaacacgtagagaaaccc

SEQ ID NO: 20 - DNA, 20 bp, synthetic construct
  source 1..20: mol_type = other DNA
gagatcagttgtctctggtt

SEQ ID NO: 21 - DNA, 23 bp, synthetic construct
  source 1..23: mol_type = other DNA
tcatgcaatgtaggtgtatctcc

SEQ ID NO: 22 - DNA, 25 bp, synthetic construct
  source 1..25: mol_type = other DNA
ctacacataataatcccactttcac

SEQ ID NO: 23 - DNA, 18 bp, synthetic construct
  source 1..18: mol_type = other DNA
aaatgccctccagtttct

SEQ ID NO: 24 - DNA, 20 bp, synthetic construct
  source 1..20: mol_type = other DNA
catctgtattgcagcctcca

SEQ ID NO: 25 - DNA, 19 bp, synthetic construct
  source 1..19: mol_type = other DNA
ccagcattaacctctaggc

SEQ ID NO: 26 - DNA, 25 bp, synthetic construct
  source 1..25: mol_type = other DNA
cgagtacgtagtgtttgtttattac

SEQ ID NO: 27 - DNA, 25 bp, synthetic construct
  source 1..25: mol_type = other DNA
attctactgtacctaatgccagtac

SEQ ID NO: 28 - DNA, 100 bp, synthetic construct
  source 1..100: mol_type = other DNA
aatgcggaacaacattagaacagcaatacaacaaaccgttgtgtgatttgttaattaggtgtattaactgtcaaaagccactgtgtcctgaagaatcact

SEQ ID NO: 29 - DNA, 107 bp, synthetic construct
  source 1..107: mol_type = other DNA
aatgcgagagggtgttagtgaaagacacactatatgccaaacaccacttacaaatattttaaatgtactaaaaactagtaatgcaaaggcagcaatgttagctcact

SEQ ID NO: 30 - DNA, 75 bp, synthetic construct
  source 1..75: mol_type = other DNA
aatgcgtaataaatcaacgtgttgcgattggtgtattgctgcatttggacttacacccagtatagctgactcact

SEQ ID NO: 31 - DNA, 96 bp, synthetic construct
  source 1..96: mol_type = other DNA
aatgcggcattagtggccatcctttattaaataaattggatgacacagaaaatgctagtgcttatgcagcaaatgcaggtgtggataatagtcact

SEQ ID NO: 32 - DNA, 95 bp, synthetic construct
  source 1..95: mol_type = other DNA
aatgcagttctatgtcagcaactatggtttaaacttgtacgtttcctgcttgccatgcgtgccaaatccctgttttcctgacctgcactgtcact

SEQ ID NO: 33 - DNA, 79 bp, synthetic construct
  source 1..79: mol_type = other DNA
aatgcctaacattaatgctggtacagattctaggtggccttatttacataatagattggtggtgtttacatttctcact

SEQ ID NO: 34 - DNA, 105 bp, synthetic construct
  source 1..105: mol_type = other DNA
aatgcagaacacgtagagaaacccagctgtaatcatgcatggagatacacctacattgcatgaatatatgttagatttgcaaccagagacaactgatctctcact

SEQ ID NO: 35 - DNA, 86 bp, synthetic construct
  source 1..86: mol_type = other DNA
aatgcctacacataataatcccactttcactgacccatctgtattgcagcctccaacacctgcagaaactggagggcattttcact

SEQ ID NO: 36 - DNA, 81 bp, synthetic construct
  source 1..81: mol_type = other DNA
aatgcccagcattaacctctaggcgtactggcattaggtacagtagaattggtaataaacaaacactacgtactcgtcact

SEQ ID NO: 37 - DNA, 21 bp, synthetic construct
  source 1..21: mol_type = other DNA
gacctccactgttatgagcaa

SEQ ID NO: 38 - DNA, 15 bp, synthetic construct
  source 1..15: mol_type = other DNA
tacccgacagctcag

SEQ ID NO: 39 - DNA, 22 bp, synthetic construct
  source 1..22: mol_type = other DNA
ctatgacatcctcctcatctga

SEQ ID NO: 40 - DNA, 21 bp, synthetic construct
  source 1..21: mol_type = other DNA
aggtcggtgtatgtcctgttg

SEQ ID NO: 41 - DNA, 17 bp, synthetic construct
  source 1..17: mol_type = other DNA
acgtgttggtttccaac

SEQ ID NO: 42 - DNA, 20 bp, synthetic construct
  source 1..20: mol_type = other DNA
tgattacacctcggtttctc

SEQ ID NO: 43 - DNA, 20 bp, synthetic construct
  source 1..20: mol_type = other DNA
cgggatactctgcgacaact

SEQ ID NO: 44 - DNA, 21 bp, synthetic construct
  source 1..21: mol_type = other DNA
cagcagctgtttatggactca

SEQ ID NO: 45 - DNA, 20 bp, synthetic construct
  source 1..20: mol_type = other DNA
cacaccacggacacacaaat

SEQ ID NO: 46 - DNA, 20 bp, synthetic construct
  source 1..20: mol_type = other DNA
accacaatactatggcgcgc

SEQ ID NO: 47 - DNA, 16 bp, synthetic construct
  source 1..16: mol_type = other DNA
tgaggatccaacacgg

SEQ ID NO: 48 - DNA, 20 bp, synthetic construct
  source 1..20: mol_type = other DNA
ggtagcttgtagggtcgccg

SEQ ID NO: 49 - DNA, 20 bp, synthetic construct
  source 1..20: mol_type = other DNA
aaactgcactgtgacgtgta

SEQ ID NO: 50 - DNA, 17 bp, synthetic construct
  source 1..17: mol_type = other DNA
aaacgccatgagaggac

SEQ ID NO: 51 - DNA, 20 bp, synthetic construct
  source 1..20: mol_type = other DNA
ctttaacgttggcttgtgtc

SEQ ID NO: 52 - DNA, 117 bp, synthetic construct
  source 1..117: mol_type = other DNA
aatgctgtgagaaacacaccacaatactatggcgcgctttgaggatccaacacggcgaccctacaagctacctgatctgtgcacggaactgaacacttcactgcaagacatatcact

SEQ ID NO: 53 - DNA, 126 bp, synthetic construct
  source 1..126: mol_type = other DNA
aatgcgaggcaactgacctccactgttatgagcaattacccgacagctcagatgaggaggatgtcatagacagtccagctggacaagcagaaccggacacatccaattacaatatcgttactcact

SEQ ID NO: 54 - DNA, 132 bp, synthetic construct
  source 1..132: mol_type = other DNA
aatgcgatttcataatatttcgggtcgttgggcagggcgctgtgcggcgtgttggaggtcccgacgtagagaaactgcactgtgacgtgtaaaaacgccatgagaggacacaagccaacgttaaaggtcact

SEQ ID NO: 55 - DNA, 151 bp, synthetic construct
  source 1..151: mol_type = other DNA
aatgcaaacgattccataacatcggtggacggtggacaggtcggtgtatgtcctgttggaaaccaacacgtagagaaaccgaggtgtaatcatgcatggagaaataactacattgcaagactatgttttagatttggaacccgaggtcact

SEQ ID NO: 56 - DNA, 152 bp, synthetic construct
  source 1..152: mol_type = other DNA
aatgcgaaccacagcgtcacacaatacagtgttcgtgttgtaagtgtaacaacacactgcagctggtagtagaagcctcacgggatactctgcgacaactacagcagctgtttatggactcactaggatttgtgtgtccgtggtgtgtcact

SEQ ID NO: 57 - DNA, 150 bp, synthetic construct
  source 1..150: mol_type = other DNA
tgttttctttagtactcagaaaggttgtgtaggattgttgagagtgttggagaaggagtgactgatcttcaaccaggagatcatgttttacccatcttcaccggagagtgtggggattgtcctcactgtcactccgaggaatccaacatg

SEQ ID NO: 58 - DNA, 18 bp, synthetic construct
  source 1..18: mol_type = other DNA
agatttggacctgcgagc

SEQ ID NO: 59 - DNA, 20 bp, synthetic construct
  source 1..20: mol_type = other DNA
ggagcggctgtctccacaag

SEQ ID NO: 60 - DNA, 15 bp, synthetic construct
  source 1..15: mol_type = other DNA
ccgcgcagagccttc

SEQ ID NO: 61 - DNA, 21 bp, synthetic construct
  source 1..21: mol_type = other DNA
gcagcagcattatattggtat

SEQ ID NO: 62 - DNA, 25 bp, synthetic construct
  source 1..25: mol_type = other DNA
tattagtgaagtgtatggagacacg

SEQ ID NO: 63 - DNA, 22 bp, synthetic construct
  source 1..22: mol_type = other DNA
tttgtctttgtatccattctgg

SEQ ID NO: 64 - DNA, 18 bp, synthetic construct
  source 1..18: mol_type = other DNA
attgaactgcaactaacg

SEQ ID NO: 65 - DNA, 20 bp, synthetic construct
  source 1..20: mol_type = other DNA
agtggacattacaagacgtt

SEQ ID NO: 66 - DNA, 20 bp, synthetic construct
  source 1..20: mol_type = other DNA
gttaaatacacttcaaggct

SEQ ID NO: 67 - DNA, 17 bp, synthetic construct
  source 1..17: mol_type = other DNA
tcctgcaggtaccaatg

SEQ ID NO: 68 - DNA, 22 bp, synthetic construct
  source 1..22: mol_type = other DNA
cattacatcccgtaccctcttc

SEQ ID NO: 69 - DNA, 20 bp, synthetic construct
  source 1..20: mol_type = other DNA
gtcatctgatatagcatccc

SEQ ID NO: 70 - DNA, 22 bp, synthetic construct
  source 1..22: mol_type = other DNA
ttgatggagacatatgcaatac

SEQ ID NO: 71 - DNA, 22 bp, synthetic construct
  source 1..22: mol_type = other DNA
tgtgaagaagcatcagtaactg

SEQ ID NO: 72 - DNA, 18 bp, synthetic construct
  source 1..18: mol_type = other DNA
caacttgaccctctacca

SEQ ID NO: 73 - DNA, 25 bp, synthetic construct
  source 1..25: mol_type = other DNA
catatagactattggaaacacatgc

SEQ ID NO: 74 - DNA, 24 bp, synthetic construct
  source 1..24: mol_type = other DNA
ctctggccttgtaataaatagcac

SEQ ID NO: 75 - DNA, 25 bp, synthetic construct
  source 1..25: mol_type = other DNA
ctggtggttaatatgtttaaatccc

SEQ ID NO: 76 - DNA, 22 bp, synthetic construct
  source 1..22: mol_type = other DNA
gacaaatcttgatactgcatcc

SEQ ID NO: 77 - DNA, 20 bp, synthetic construct
  source 1..20: mol_type = other DNA
caacattactggcgtgcttt

SEQ ID NO: 78 - DNA, 21 bp, synthetic construct
  source 1..21: mol_type = other DNA
ggacgtattaataggcagaca

SEQ ID NO: 79 - DNA, 18 bp, synthetic construct
  source 1..18: mol_type = other DNA
ctgcatttaacagctcac

SEQ ID NO: 80 - DNA, 20 bp, synthetic construct
  source 1..20: mol_type = other DNA
acaaaggacggattaactgt

SEQ ID NO: 81 - DNA, 20 bp, synthetic construct
  source 1..20: mol_type = other DNA
taaatgtactatgggtgtag

SEQ ID NO: 82 - DNA, 17 bp, synthetic construct
  source 1..17: mol_type = other DNA
ggcacgcctaggattat

SEQ ID NO: 83 - DNA, 24 bp, synthetic construct
  source 1..24: mol_type = other DNA
cacaacaggttaaagttgtagacc

SEQ ID NO: 84 - DNA, 20 bp, synthetic construct
  source 1..20: mol_type = other DNA
agtttagtgggagtggttac

SEQ ID NO: 85 - DNA, 18 bp, synthetic construct
  source 1..18: mol_type = other DNA
gcacctaaagaagatccc

SEQ ID NO: 86 - DNA, 20 bp, synthetic construct
  source 1..20: mol_type = other DNA
tttctgcagacctagatcag

SEQ ID NO: 87 - DNA, 18 bp, synthetic construct
  source 1..18: mol_type = other DNA
tttgcgtcctaaaggaaa

SEQ ID NO: 88 - DNA, 17 bp, synthetic construct
  source 1..17: mol_type = other DNA
cccaccacctcatctac

SEQ ID NO: 89 - DNA, 22 bp, synthetic construct
  source 1..22: mol_type = other DNA
ctctacaactgctaaacgcaaa

SEQ ID NO: 90 - DNA, 17 bp, synthetic construct
  source 1..17: mol_type = other DNA
acacaacaaacaacact

SEQ ID NO: 91 - DNA, 22 bp, synthetic construct
  source 1..22: mol_type = other DNA
ccagaaagttaccacagttatg

SEQ ID NO: 92 - DNA, 23 bp, synthetic construct
  source 1..23: mol_type = other DNA
cacagagctgcaaacaactatac

SEQ ID NO: 93 - DNA, 18 bp, synthetic construct
  source 1..18: mol_type = other DNA
ctgttgcttgcagtacac

SEQ ID NO: 94 - DNA, 17 bp, synthetic construct
  source 1..17: mol_type = other DNA
ctctacgcttcggttgt

SEQ ID NO: 95 - DNA, 26 bp, synthetic construct
  source 1..26: mol_type = other DNA
cgtagacattcgtactttggaagacc

SEQ ID NO: 96 - DNA, 19 bp, synthetic construct
  source 1..19: mol_type = other DNA
tcctagtgtgcccattaac

SEQ ID NO: 97 - DNA, 21 bp, synthetic construct
  source 1..21: mol_type = other DNA
cacgtagacattcgtactttg

SEQ ID NO: 98 - DNA, 23 bp, synthetic construct
  source 1..23: mol_type = other DNA
caattcctagtgtgcccattaac

SEQ ID NO: 99 - DNA, 24 bp, synthetic construct
  source 1..24: mol_type = other DNA
gtagattatggtttctgagaacag

SEQ ID NO: 100 - DNA, 21 bp, synthetic construct
  source 1..21: mol_type = other DNA
gtatgtcttgttgcagatcat

SEQ ID NO: 101 - DNA, 21 bp, synthetic construct
  source 1..21: mol_type = other DNA
agaacacgtagagaaacccag

SEQ ID NO: 102 - DNA, 19 bp, synthetic construct
  source 1..19: mol_type = other DNA
tgtaggtgtatctccatgc

SEQ ID NO: 103 - DNA, 17 bp, synthetic construct
  source 1..17: mol_type = other DNA
gctcagaggaggaggat

SEQ ID NO: 104 - DNA, 21 bp, synthetic construct
  source 1..21: mol_type = other DNA
cggacagagcccattacaata

SEQ ID NO: 105 - DNA, 19 bp, synthetic construct
  source 1..19: mol_type = other DNA
gagtcacacttgcaacaaa

SEQ ID NO: 106 - DNA, 22 bp, synthetic construct
  source 1..22: mol_type = other DNA
gagcaattaaatgacagctcag

SEQ ID NO: 107 - DNA, 23 bp, synthetic construct
  source 1..23: mol_type = other DNA
ctggaccatctatttcatcctcc

SEQ ID NO: 108 - DNA, 17 bp, synthetic construct
  source 1..17: mol_type = other DNA
gtccggttctgcttgtc

SEQ ID NO: 109 - DNA, 21 bp, synthetic construct
  source 1..21: mol_type = other DNA
cacgtagacattcgtactttg

SEQ ID NO: 110 - DNA, 23 bp, synthetic construct
  source 1..23: mol_type = other DNA
caattcctagtgtgcccattaac

SEQ ID NO: 111 - DNA, 21 bp, synthetic construct
  source 1..21: mol_type = other DNA
gccatggtagattatggtttc

SEQ ID NO: 112 - DNA, 21 bp, synthetic construct
  source 1..21: mol_type = other DNA
gagatgcagtacaggttctaa

SEQ ID NO: 113 - DNA, 22 bp, synthetic construct
  source 1..22: mol_type = other DNA
acgaaagtatttgggtagtcca

SEQ ID NO: 114 - DNA, 25 bp, synthetic construct
  source 1..25: mol_type = other DNA
gtctacacatccactaatatcacta

SEQ ID NO: 115 - DNA, 20 bp, synthetic construct
  source 1..20: mol_type = other DNA
cattcgtactttggaagacc

SEQ ID NO: 116 - DNA, 23 bp, synthetic construct
  source 1..23: mol_type = other DNA
caattcctagtgtgcccattaac

SEQ ID NO: 117 - DNA, 24 bp, synthetic construct
  source 1..24: mol_type = other DNA
gtagattatggtttctgagaacag

SEQ ID NO: 118 - DNA, 22 bp, synthetic construct
  source 1..22: mol_type = other DNA
caacatagagatgcagtacagg

SEQ ID NO: 119 - DNA, 25 bp, synthetic construct
  source 1..25: mol_type = other DNA
cgaaagtatttgggtagtccactta

SEQ ID NO: 120 - DNA, 20 bp, synthetic construct
  source 1..20: mol_type = other DNA
ttgtctacacatccactaat

SEQ ID NO: 121 - DNA, 17 bp, synthetic construct
  source 1..17: mol_type = other DNA
tgcaagtgcctttctaa

SEQ ID NO: 122 - DNA, 20 bp, synthetic construct
  source 1..20: mol_type = other DNA
aggattgtgcaacaatgtgt

SEQ ID NO: 123 - DNA, 20 bp, synthetic construct
  source 1..20: mol_type = other DNA
tctgctcgtttataatgtct

SEQ ID NO: 124 - DNA, 21 bp, synthetic construct
  source 1..21: mol_type = other DNA
gacatagtagacgatagtgaa

SEQ ID NO: 125 - DNA, 25 bp, synthetic construct
  source 1..25: mol_type = other DNA
cagacactaatagtaatgcaagtgc

SEQ ID NO: 126 - DNA, 17 bp, synthetic construct
  source 1..17: mol_type = other DNA
tttgcctgtgaattact

SEQ ID NO: 127 - DNA, 17 bp, synthetic construct
  source 1..17: mol_type = other DNA
cgggtggtcaggtaata

SEQ ID NO: 128 - DNA, 23 bp, synthetic construct
  source 1..23: mol_type = other DNA
ctacatctgtgtttagcagcaac

SEQ ID NO: 129 - DNA, 19 bp, synthetic construct
  source 1..19: mol_type = other DNA
ccaagtgctgcctaataat

SEQ ID NO: 130 - DNA, 22 bp, synthetic construct
  source 1..22: mol_type = other DNA
gttcatgaaggaatacgaacat

SEQ ID NO: 131 - DNA, 22 bp, synthetic construct
  source 1..22: mol_type = other DNA
ctgcatcatctttaaactgcac

SEQ ID NO: 132 - DNA, 22 bp, synthetic construct
  source 1..22: mol_type = other DNA
gcatgaacttcccatactttat

SEQ ID NO: 133 - DNA, 17 bp, synthetic construct
  source 1..17: mol_type = other DNA
tacaggcggacgcactg

SEQ ID NO: 134 - DNA, 23 bp, synthetic construct
  source 1..23: mol_type = other DNA
agatacacttgctcctgtaagac

SEQ ID NO: 135 - DNA, 21 bp, synthetic construct
  source 1..21: mol_type = other DNA
cccacaggatctactgttaaa

SEQ ID NO: 136 - DNA, 17 bp, synthetic construct
  source 1..17: mol_type = other DNA
acaatgcgacacaaacg

SEQ ID NO: 137 - DNA, 22 bp, synthetic construct
  source 1..22: mol_type = other DNA
tataaagttgggtagccgatgc

SEQ ID NO: 138 - DNA, 17 bp, synthetic construct
  source 1..17: mol_type = other DNA
ctgcctgtttgcatgtt

SEQ ID NO: 139 - DNA, 18 bp, synthetic construct
  source 1..18: mol_type = other DNA
gacacaaacgttctgcaa

SEQ ID NO: 140 - DNA, 22 bp, synthetic construct
  source 1..22: mol_type = other DNA
tataaagttgggtagccgatgc

SEQ ID NO: 141 - DNA, 17 bp, synthetic construct
  source 1..17: mol_type = other DNA
acatgtacctgcctgtt

SEQ ID NO: 142 - DNA, 19 bp, synthetic construct
  source 1..19: mol_type = other DNA
attccattgggaacaaggc

SEQ ID NO: 143 - DNA, 23 bp, synthetic construct
  source 1..23: mol_type = other DNA
agatacacttgctcctgtaagac

SEQ ID NO: 144 - DNA, 17 bp, synthetic construct
  source 1..17: mol_type = other DNA
gggcccacaggatctac

SEQ ID NO: 145 - DNA, 21 bp, synthetic construct
  source 1..21: mol_type = other DNA
cactgggtatattccattggg

SEQ ID NO: 146 - DNA, 23 bp, synthetic construct
  source 1..23: mol_type = other DNA
agatacacttgctcctgtaagac

SEQ ID NO: 147 - DNA, 17 bp, synthetic construct
  source 1..17: mol_type = other DNA
atcagaagggcccacag

SEQ ID NO: 148 - DNA, 22 bp, synthetic construct
  source 1..22: mol_type = other DNA
aggatggtgatatggttgatac

SEQ ID NO: 149 - DNA, 24 bp, synthetic construct
  source 1..24: mol_type = other DNA
ctttggtgctatggactttactac

SEQ ID NO: 150 - DNA, 21 bp, synthetic construct
  source 1..21: mol_type = other DNA
tatccagtggaacttcacttt

SEQ ID NO: 151 - DNA, 22 bp, synthetic construct
  source 1..22: mol_type = other DNA
ctttactacattacaggctaac

SEQ ID NO: 152 - DNA, 22 bp, synthetic construct
  source 1..22: mol_type = other DNA
caaatatccagtggaacttcac

SEQ ID NO: 153 - DNA, 17 bp, synthetic construct
  source 1..17: mol_type = other DNA
tggttctgacaccattt

SEQ ID NO: 154 - DNA, 28 bp, synthetic construct
  source 1..28: mol_type = other DNA
cagggtatttagaatacatttacctgac

SEQ ID NO: 155 - DNA, 22 bp, synthetic construct
  source 1..22: mol_type = other DNA
tataatccagatacacagcggc

SEQ ID NO: 156 - DNA, 17 bp, synthetic construct
  source 1..17: mol_type = other DNA
tacacaggcccaaacca

SEQ ID NO: 157 - DNA, 20 bp, synthetic construct
  source 1..20: mol_type = other DNA
ttataatccagatacacagc

SEQ ID NO: 158 - DNA, 21 bp, synthetic construct
  source 1..21: mol_type = other DNA
ctgtgtaggtgttgaggtagg

SEQ ID NO: 159 - DNA, 18 bp, synthetic construct
  source 1..18: mol_type = other DNA
ctaatgcccacacctaat

SEQ ID NO: 160 - DNA, 20 bp, synthetic construct
  source 1..20: mol_type = other DNA
gttcctaaagtatcaggatt

SEQ ID NO: 161 - DNA, 30 bp, synthetic construct
  source 1..30: mol_type = other DNA
cagggtatttagaatacatttacctgaccc

SEQ ID NO: 162 - DNA, 17 bp, synthetic construct
  source 1..17: mol_type = other DNA
aaatgaggtgtcaggaa

SEQ ID NO: 163 - DNA, 19 bp, synthetic construct
  source 1..19: mol_type = other DNA
tgcaaatttagccagttca

SEQ ID NO: 164 - DNA, 25 bp, synthetic construct
  source 1..25: mol_type = other DNA
ctacacctagtggttctatggttac

SEQ ID NO: 165 - DNA, 20 bp, synthetic construct
  source 1..20: mol_type = other DNA
gctcgttgtaaccaataagg

SEQ ID NO: 166 - DNA, 20 bp, synthetic construct
  source 1..20: mol_type = other DNA
cattaaaggctctgggtcta

SEQ ID NO: 167 - DNA, 20 bp, synthetic construct
  source 1..20: mol_type = other DNA
ctgcaaatttagccagttca

SEQ ID NO: 168 - DNA, 20 bp, synthetic construct
  source 1..20: mol_type = other DNA
gaaccactaggtgtaggaaa

SEQ ID NO: 169 - DNA, 18 bp, synthetic construct
  source 1..18: mol_type = other DNA
atgtttcaggacccacag

SEQ ID NO: 170 - DNA, 23 bp, synthetic construct
  source 1..23: mol_type = other DNA
ccagaaagttaccacagttatgc

SEQ ID NO: 171 - DNA, 22 bp, synthetic construct
  source 1..22: mol_type = other DNA
catgtatagttgtttgcagctc

SEQ ID NO: 172 - DNA, 22 bp, synthetic construct
  source 1..22: mol_type = other DNA
ggttacacatttacaagcaact

SEQ ID NO: 173 - DNA, 20 bp, synthetic construct
  source 1..20: mol_type = other DNA
actaagggcgtaaccgaaat

SEQ ID NO: 174 - DNA, 19 bp, synthetic construct
  source 1..19: mol_type = other DNA
ctaaccggtttcggttcaa

SEQ ID NO: 175 - DNA, 18 bp, synthetic construct
  source 1..18: mol_type = other DNA
cgctgttaggcacatatt

SEQ ID NO: 176 - DNA, 24 bp, synthetic construct
  source 1..24: mol_type = other DNA
ccttagaagtttaaaccttatgcc

SEQ ID NO: 177 - DNA, 21 bp, synthetic construct
  source 1..21: mol_type = other DNA
tgaactagggtgacatttagt

SEQ ID NO: 178 - DNA, 17 bp, synthetic construct
  source 1..17: mol_type = other DNA
cgccaacgccttacata

SEQ ID NO: 179 - DNA, 20 bp, synthetic construct
  source 1..20: mol_type = other DNA
ccgctgttaggcacatattt

SEQ ID NO: 180 - DNA, 25 bp, synthetic construct
  source 1..25: mol_type = other DNA
ccttagaagtttaaaccttatgcca

SEQ ID NO: 181 - DNA, 20 bp, synthetic construct
  source 1..20: mol_type = other DNA
gatcttcaaccaggagatca

SEQ ID NO: 182 - DNA, 20 bp, synthetic construct
  source 1..20: mol_type = other DNA
agtgacagtgaggacaatcc

SEQ ID NO: 183 - DNA, 16 bp, synthetic construct
  source 1..16: mol_type = other DNA
acccatcttcaccgga